(19) United States
(12) Patent Application Publication
(10) Pub. No.: US 2024/0082311 A1
(43) Pub. Date: Mar. 14, 2024

(54) COMPOSITIONS AND METHODS FOR TREATING SPINAL CORD INJURIES

(71) Applicant: ASTERIAS BIOTHERAPEUTICS, INC., Carlsbad, CA (US)

(72) Inventors: Francois BINETTE, Napa, CA (US); Jennifer BAHR-DAVIDSON, Carlsbad, CA (US); Rami SKALITER, Ness Ziona (IL); Kento ONISHI, Oakland, CA (US); Nathan C. MANLEY, San Jose, CA (US); Craig R. HALBERSTADT, Pleasanton, CA (US); Erik M. WHITELEY, Concord, CA (US)

(21) Appl. No.: 18/262,909

(22) PCT Filed: Jan. 28, 2022
(86) PCT No.: PCT/US22/14373

§ 371 Date: Jul. 25, 2023

Related U.S. Application Data
(60) Provisional application No. 63/182,760, filed on Apr. 30, 2021.
(60) Provisional application No. 63/142,935, filed on Jan. 28, 2021.

(51) Int. Cl. A61K 35/30; A61K 9/00; A61P 25/00; C12N 5/079

(52) U.S. Cl. CPC A61K 35/30; A61K 9/0019; A61K 9/0085; A61P 25/00; C12N 5/0622; C12N 2500/38; C12N 2501/155; C12N 2501/727; C12N 2506/02

(57) ABSTRACT

Provided herein are methods, compositions of matter, and devices for treating neurological diseases and illnesses, including spinal cord injury.

### BACKGROUND

Spinal cord injury (SCI) is a devastating and currently untreatable condition, aside from symptomatic treatments for some of the resulting complications. Spinal cord injury results in complete or partial loss of motor, sensory, and autonomic function. As a result, patients often lose mobility and may be wheelchair-bound, in addition to suffering numerous medical complications. Over 12,000 Americans suffer a spinal cord injury (SCI) each year, and approximately 1.3 million people in the United States are estimated to be living with a spinal cord injury. Traumatic SCI most commonly impacts individuals in their twenties and thirties, resulting in a high-level of permanent disability in young and previously healthy individuals. Individuals with SCI not only have impaired limb function, but suffer from impaired bowel and bladder function, reduced sensation, spasticity, autonomic dysreflexia, thromboses, sexual dysfunction, increased infections, decubitus ulcers and chronic pain, which can each significantly impact quality of life, and can even be life threatening in some instances. The life expectancy of an individual suffering a cervical spinal cord injury at age 20 is 20-25 years lower than that of a similarly aged individual with no SCI (NSCISC Spinal Cord Injury Facts and Figures 2013). To date, there are no treatments approved by the United States Food and Drug Administration (FDA) to induce neurological recovery following spinal cord injury (SCI). Several interventions including glucocorticoids, modulation of voltage-gated channels, tetracycline antibiotics, and cell-based therapies have been studied in clinical trials, however, none to date have met critical registration endpoints.

The clinical effects of spinal cord injury vary with the site and extent of damage. The neural systems that may be permanently disrupted below the level of the injury not only involve loss of control of limb muscles and the protective roles of temperature and pain sensation, but impact the cardiovascular system, breathing, sweating, bowel control, bladder control, and sexual function (Anderson K D, Friden J, Lieber RL. Acceptable benefits and risks associated with surgically improving arm function in individuals living with cervical spinal cord injury. Spinal Cord. 2009 April; 47(4):334-8.) These losses lead to a succession of secondary problems, such as pressure sores and urinary tract infections that, until modern medicine, were rapidly fatal. Spinal cord injury often removes those unconscious control mechanisms that maintain the appropriate level of excitability in neural circuitry of the spinal cord. As a result, spinal motoneurons can become spontaneously hyperactive, producing debilitating stiffness and uncontrolled muscle spasms or spasticity. This hyperactivity can also cause sensory systems to produce chronic neurogenic pain and paresthesias, unpleasant sensations including numbness, tingling, aches, and burning. In recent polls of spinal cord injury patients, recovery of ambulatory function was not the highest ranked function that these patients desired to regain, but in many cases, relief from the spontaneous hyperactivity sequelae was paramount (Anderson K D, Friden J, Lieber RL. Acceptable benefits and risks associated with surgically improving arm function in individuals living with cervical spinal cord injury. Spinal Cord. 2009 April; 47(4):334-38).

There exists a need for treatments for spinal cord injury, and related pathologies.

### SUMMARY

The examples and embodiments presented herein describe human embryonic stem cell (hESC) derived cells for the treatment of spinal cord injuries (SCI) as described in greater detail herein.

For example, an OPC composition obtained in accordance with the present disclosure can be used in cellular therapy to improve one or more neurological functions in a subject in need of treatment. In an embodiment, an OPC cell population in accordance with the present disclosure can be injected, implanted, or otherwise delivered into a subject in need thereof. In an embodiment, a cell population in accordance with the present disclosure can be implanted or otherwise delivered into a subject in need thereof for treating spinal cord injury, stroke, or multiple sclerosis.

The LCTOPC1 is a cell population containing a mixture of oligodendrocyte progenitor cells and other characterized cell types obtained following directed differentiation of an established and well-characterized line of hESC. AST-OPC1 (formerly known as GRNOPC1) is a cell population that contains a mixture of oligodendrocyte progenitor cells (OPCs) and other characterized cell types that are obtained following differentiation of undifferentiated human embryonic stem cells (uhESCs). Oligodendrocyte progenitor cells (OPCs) are a subtype of glial cells in the central nervous system (CNS) that arise in the ventricular zones of the brain and spinal cord and migrate throughout the developing CNS before maturing into oligodendrocytes. Mature oligodendrocytes produce the myelin sheath that insulates neuronal axons and remyelinate CNS lesions where the myelin sheath has been lost. Oligodendrocytes also contribute to neuroprotection through other mechanisms, including production of neurotrophic factors that promote neuronal survival (Wilkins et al., 2001 Glia 36(1):48-57; Dai et al., 2003 J Neurosci. 23(13):5846-53; Du and Dreyfus, 2002 J Neurosci Res. 68(6):647-54). Unlike most progenitor cells, OPCs remain abundant in the adult CNS where they retain the ability to generate new oligodendrocytes. Accordingly, OPCs and mature oligodendrocytes derived from OPCs are an important therapeutic target for demyelinating and dysmyelinating disorders (such as multiple sclerosis, adrenoleukodystrophy and adrenomyeloneuropathy), other neurodegenerative disorders (such as Alzheimer's disease, amyotrophic lateral sclerosis, and Huntington's disease) and acute neurological injuries (such as stroke and spinal cord injury (SCI)).

An OPC composition obtained in accordance with the present disclosure can be used in cellular therapy to improve one or more neurological functions in a subject in need of treatment. In an embodiment, an OPC cell population in accordance with the present disclosure can be injected or implanted into a subject in need thereof. In an embodiment, a cell population in accordance with the present disclosure can be implanted into a subject in need thereof for treating spinal cord injury, stroke, or multiple sclerosis.

In certain embodiments, the OPC1 composition is administered after the subject has suffered a traumatic spinal cord injury. In some embodiments, the OPC1 composition is administered between 14-90 days after the spinal cord injury, such as between 14-75 days after the spinal cord injury, such as between 14-60 days after the spinal cord injury, such as between 14-30 days after the injury, such as between 20-75 days after the injury, such as between 20-60 days after the injury, and such as between 20-40 days after the injury. In certain embodiments, the OPC1 composition is administered about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 days after the injury. In certain embodiments, the OPC1 composition is administered between 14 days and the lifetime of the subject.

Methods and compositions for obtaining a population of cells comprising dorsal neural progenitor cells (dNPCs) from undifferentiated human pluripotent stem cells can be found in WO/2020/154533, WO/2020/061371, U.S. Pat. No. 10,286,009, WO/2017/031092, WO/2017/173064 and WO/2018/053210, each of which are incorporated by reference in their entirety for all methods, compositions, cells, data, definitions, uses, and all other information provided therein.

In an aspect, a method of improving one or more neurological functions in a subject having a spinal cord injury (SCI) is provided, the method including: administering to the subject a first dose of a composition including human pluripotent stem cell-derived oligodendrocyte progenitor cells (OPCs); and optionally administering two or more doses of the composition.

In some embodiments, the method further includes administering to the subject a second dose of the composition. In some embodiments, the method further includes administering to the subject a third dose of the composition. In some embodiments, each administration includes delivering, for example by injection, the composition into the spinal cord of the subject. In some embodiments, each administration includes delivering two or more fractions of a dose. In some embodiments, the SCI is a subacute cervical SCI. In some embodiments, the SCI is a chronic cervical SCI. In some embodiments, the SCI is a subacute thoracic SCI. In some embodiments, the SCI is a chronic thoracic SCI. In some embodiments, the first dose, second dose, and/or third dose of the composition includes about 1×10⁶ to about 3×10⁷ OPC cells. In some embodiments, the first dose of the composition includes about 2×10⁶ OPC cells. In some embodiments, the first dose or the second dose of the composition includes about 1×10⁷ OPC cells. In some embodiments, the second dose or the third dose of the composition includes about 2×10⁷ OPC cells. In some embodiments, each of the first dose, second dose, and third dose of the composition are administered about 20 to about 45 days after the SC. In some embodiments, each of the first dose, second dose, and third dose of the composition are administered about 14 to about 90 days after the SCI. In some embodiments, each of the first dose, second dose, and third dose of the composition are administered about 14 to about 75 days after the SCI. In some embodiments, each of the first dose, second dose, and third dose of the composition are administered about 14 to about 60 days after the SCI. In some embodiments, each of the first dose, second dose, and third dose of the composition are administered about 14 to about 30 days after the SCI. In some embodiments, each of the first dose, second dose, and third dose of the composition are administered about 20 to about 75 days after the SCI. In some embodiments, each of the first dose, second dose, and third dose of the composition are administered about 20 to about 60 days after the SCI. In some embodiments, each of the first dose, second dose, and third dose of the composition are administered about 20 to about 40 days after the SC. In some embodiments, each of the first dose, second dose, and third dose of the composition are administered between about 14 days after the SCI and the lifetime of the subject. In some embodiments, the injection is performed in a caudal half of an epicenter of the SCI. In some embodiments, the injection is about 6 mm into the spinal cord of the subject. In some embodiments, the injection is about 5 mm into the spinal cord of the subject.

In another, aspect, a method of improving one or more neurological functions in a subject having a spinal cord injury (SCI) is provided, the method including: administering to the subject a dose of a composition including human pluripotent stem cell-derived oligodendrocyte progenitor cells (OPCs).

In some embodiments, the dose of the composition includes about 1×10⁶ to about 3×10⁷ OPC cells. In some embodiments, the dose of the composition includes about 2×10⁶ OPC cells. In some embodiments, the administration of the composition includes injecting, implanting, or otherwise delivering the composition into the spinal cord of the subject. In some embodiments, the dose of the composition is administered about 7 to about 14 days after the SCI. In some embodiments, the injection is performed in a caudal half of an epicenter of the SCI. In some embodiments, the injection is about 6 mm into the spinal cord of the subject. In some embodiments, the injection is about 5 mm into the spinal cord of the subject. In some embodiments, the SCI is a subacute thoracic SCI. In some embodiments, the SCI is a chronic thoracic SC. In some embodiments, the SCI is a subacute cervical SCI. In some embodiments, the SCI is a chronic cervical SCI. In some embodiments, improving one or more neurological functions includes an improvement in ISNCSCI exam upper extremity motor score (UEMS). In some embodiments, the improvement in UEMS occurs within about 6 months, about 12 months, about 18 months, about 24 months or more after injection. In some embodiments, the improvement is an increase in UEMS of at least 10%, compared to baseline. In some embodiments, improving one or more neurological functions includes an improvement in lower extremity motor scores (LEMS). In some embodiments, the improvement in LEMS occurs within about 6 months, about 12 months, about 18 months, about 24 months or more after injection. In some embodiments, the improvement is at least one motor level improvement. In some embodiments, the improvement is at least two motor level improvement. In some embodiments, the improvement is on one side of the subject's body. In some embodiments, the improvement is on both sides of the subject's body. In some embodiments, the dose of the composition is administered about 14 to 90 days after the SCI. In some embodiments, the dose of the composition is administered about 14 to about 75 days after the SCI. In some embodiments, the dose of the composition is administered about 14 to about 60 days after the SCI. In some embodiments, the dose of the composition is administered about 14 to about 30 days after the SCI. In some embodiments, the dose of the composition is administered about 20 to about 75 days after the SCI. In some embodiments, the dose of the composition is administered about 20 to about 60 days after the SCI. In some embodiments, the dose of the composition is administered about 20 to about 40 days after the SCI. In some embodiments, the dose of the composition is administered between about 14 days after the SCI and the lifetime of the subject.

In another, aspect, a cell population is provided, the cell population including an increased proportion of cells positive for oligodendrocyte progenitor cell marker NG2 and reduced expression of non-OPC markers CD49f, CLDN6, and EpCAM, wherein the cell population is prepared according to the following method: culturing undifferentiated human embryonic stem cells (uhESC) in Glial Progenitor Medium including a MAPK/ERK inhibitor, a BMP signaling inhibitor, and Retinoic Acid to obtain glial-restricted cells; differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

In some embodiments, the cell population is used in treating a thoracic spinal cord injury (SCI) in a subject. In some embodiments, the thoracic SCI is a subacute thoracic SCI. In some embodiments, the thoracic SCI is a chronic thoracic SCI. In some embodiments, the cell population is used in treating a cervical spinal cord injury (SCI) in a subject. In some embodiments, the cervical SCI is a subacute cervical SCI. In some embodiments, the cervical SCI is a chronic cervical SCI. In some embodiments, the composition is administered by implantation or other delivery method. In some embodiments, the composition is administered via injection to the subject after the SCI. In some embodiments, the injection is performed in a caudal half of an epicenter of the SCI. In some embodiments, the injection is about 6 mm into the spinal cord of the subject. In some embodiments, the injection is about 5 mm into the spinal cord of the subject. In some embodiments, the injection is performed about 14 to about 90 days after the SCI. In some embodiments, the injection is performed about 14 to about 75 days after the SCI. In some embodiments, the injection is performed about 14 to about 60 days after the SCI. In some embodiments, the injection is performed about 14 to about 30 days after the SCI. In some embodiments, the injection is performed about 20 to about 75 days after the SCI. In some embodiments, the injection is performed about 20 to about 60 days after the SCI. In some embodiments, the injection is performed about 20 to about 40 days after the SCI. In some embodiments, the injection is performed between about 14 days after the SCI and the lifetime of the subject.

In another, aspect, a method of improving one or more neurological functions in a subject having a spinal cord injury (SCI) is provided, the method including: administering to the subject a first dose of the cell population of claim 54; administering to the subject a second dose of the cell population; and optionally administering to the subject a third dose of the cell population.

In some embodiments, the SCI is a subacute cervical SCI. In some embodiments, the SCI is a chronic cervical SCI. In some embodiments, the SCI is a subacute thoracic SCI. In some embodiments, the SCI is a chronic thoracic SCI. In some embodiments, each of the first dose, second dose, and third dose of the composition are administered about 14 to about 90 days after the SCI. In some embodiments, each of the first dose, second dose, and third dose of the composition are administered about 14 to about 75 days after the SCI. In some embodiments, each of the first dose, second dose, and third dose of the composition are administered about 14 to about 60 days after the SCI. In some embodiments, each of the first dose, second dose, and third dose of the composition are administered about 14 to about 30 days after the SCI. In some embodiments, each of the first dose, second dose, and third dose of the composition are administered about 20 to about 75 days after the SCI. In some embodiments, each of the first dose, second dose, and third dose of the composition are administered about 20 to about 60 days after the SCI. In some embodiments, each of the first dose, second dose, and third dose of the composition are administered about 20 to about 40 days after the SCI. In some embodiments, each of the first dose, second dose, and third dose of the composition are administered between about 14 days after the SCI and the lifetime of the subject.

### BRIEF DESCRIPTION OF THE DRAWINGS

Embodiments will now be described, by way of example only, with reference to the accompanying drawings, in which:

FIG. 1 is a Phase-1 clinical trial schematic timeline.

FIG. 2 is schematic for patient screening, treatment, and follow-up during a Phase-1 clinical trial (CONSORT flow diagram).

FIG. 3 is a diagram illustrating the International Standards for Neurological Classification of Spinal Cord Injury (ISNCSCI) screening and at 5-year follow-up (* one ISNSCI performed at 4-year). In the figure green denotes normal motor and/or sensation, red absent motor and/or sensation, orange and light red represent sensation that is present but abnormal.

FIG. 4 is an example questionnaire administered for the long-term protocol, annual visits were required in years 2-5. Subsequent to the year 5 annual visit, follow-up was by annual phone questionnaires.

FIG. 5 is a study schematic of subjects.

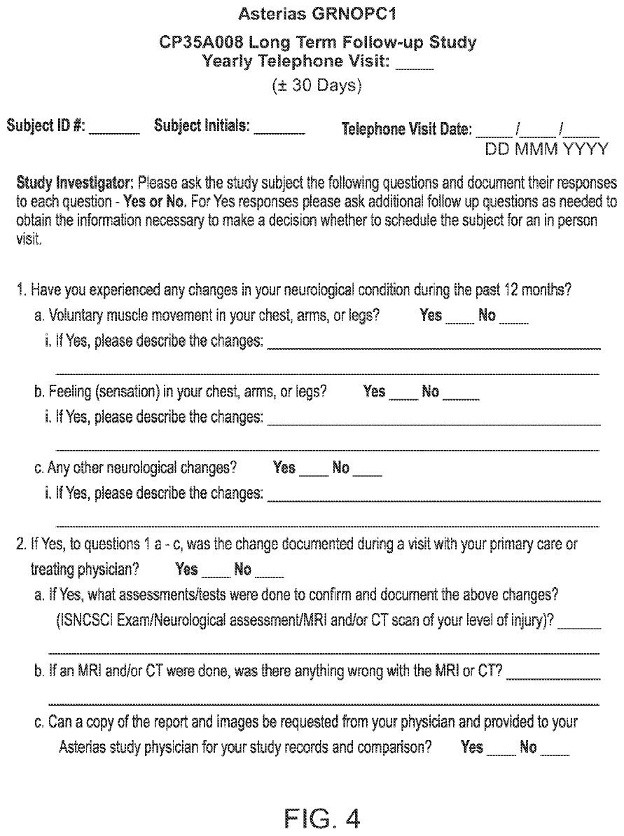

FIG. 6 is a clinical trial schematic timeline.

FIG. 7 is a schematic for patient screening and treatment during a clinical trial.

FIG. 8 is a schematic of the cohort structure and enrollment progression of a clinical trial consistent with the implementations of the present disclosure.

FIG. 9 is another Phase-1 clinical trial schematic timeline consistent with implementations of the present disclosure.

FIG. 10 is an overview of two example cell manufacturing processes consistent with implementations of the present disclosure.

FIG. 11 is a flow chart of a signaling sequence schematic of a cell differentiation process consistent with implementations of the present disclosure.

FIG. 12 is a flowchart of a production process flow consistent with implementations of the present disclosure.

### DETAILED DESCRIPTION

Before the present compositions and methods are described, it is to be understood that the present disclosure is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “AST-OPC1” refers to a specific, characterized, in vitro differentiated cell population containing a mixture of oligodendrocyte progenitor cells (OPCs) and other characterized cell types obtained from undifferentiated human embryonic stem cells (uhESCs) according to specific differentiation protocols disclosed herein.

Compositional analysis of AST-OPC1 by immunocytochemistry (ICC), flow cytometry, and quantitative polymerase chain reaction (qPCR) demonstrates that the cell population is comprised primarily of neural lineage cells of the oligodendrocyte phenotype. Other neural lineage cells, namely astrocytes and neurons, are present at low frequencies. The only non-neural cells detected in the population are epithelial cells. Mesodermal, endodermal lineage cells and uhESCs are routinely below quantitation or detection of the assays.

The term “oligodendrocyte progenitor cells” (OPCs), as used herein, refers to cells of neuroectoderm/glial lineage having the characteristics of a cell type found in the central nervous system, capable of differentiating into oligodendrocytes. These cells typically express the characteristic markers Nestin, NG2 and PDGF-Ra.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

The term “subject,” as used herein includes, but is not limited to, humans, nonhuman primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

As used herein, “implantation” or “transplantation” refers to the administration of a cell population into a target tissue using a suitable delivery technique, (e.g., using an injection device, implantation device, or other delivery device).

As used herein, “engraftment” and “engrafting” refer to incorporation of implanted tissue or cells (i.e. “graft tissue” or “graft cells”) into the body of a subject. The presence of graft tissue or graft cells at or near the implantation site 180 days or later, post implantation, is indicative of engraftment. In certain embodiments, imaging techniques (such as, e.g. MRI imaging), can be used to detect the presence of graft tissue.

As used herein, “allogeneic” and “allogeneically derived” refer to cell populations derived from a source other than the subject and hence genetically non-identical to the subject. In certain embodiments, allogeneic cell populations are derived from cultured pluripotent stem cells. In certain embodiments, allogeneic cell populations are derived from hESCs. In other embodiments, allogeneic cell populations are derived from induced pluripotent stem (iPS) cells. In yet other embodiments, allogeneic cell populations are derived from primate pluripotent (pPS) cells.

As used herein, “parenchymal cavitation” refers to formation of a lesion or cavity within a CNS injury site or proximate to a CNS injury site, in an area normally occupied by parenchymal CNS tissue. The cavities or lesions can be filled with extracellular fluid and may contain macrophages, small bands of connective tissue and blood vessels.

The terms “central nervous system” and “CNS” as used interchangeably herein refer to the complex of nerve tissues that control one or more activities of the body, which include but are not limited to, the brain and the spinal cord in vertebrates.

The term ‘decorin’ as used herein refers to a proteoglycan that, in humans, is encoded by the DCN gene. Decorin is a small cellular or pericellular matrix proteoglycan, and the protein is a component of connective tissue, binds to type I collagen fibrils, and plays a role in matrix assembly.

The term ‘chronic’ as used herein includes, but is not intended to be limited to, a condition occurring in a subject over a time period occurring between 90 days after an injury and the lifetime of a subject.

The term ‘subacute’ as used herein includes, but is not intended to be limited to, a condition occurring in a subject over a time period of between 14 days and 90 days after an injury.

There are multiple pathologies observed in the injured spinal cord due to the injury itself and subsequent secondary effects due to edema, hemorrhage and inflammation (Kakulas BA. The applied neuropathology of human spinal cord injury. Spinal Cord. 1999 Feb.;37(2):79-88). These pathologies include the severing of axons, demyelination, parenchymal cavitation and the production of ectopic tissue such as fibrous scar tissue, gliosis, and dystrophic calcification (Anderson D K, Hall ED. Pathophysiology of spinal cord trauma. Ann Emerg Med. 1993 Jun.;22(6):987-92; Norenberg MD, Smith J, Marcillo A. The pathology of human spinal cord injury: defining the problems. J. Neurotrauma. 2004 Apr.;21(4):429-40). Oligodendrocytes, which provide both neurotrophic factor and myelination support for axons are susceptible to cell death following SCI and therefore are an important therapeutic target (Almad A, Sahinkaya F R, Mctigue D M. Oligodendrocyte fate after spinal cord injury. Neurotherapics 2011 8(2): 262-73). Replacement of the oligodendrocyte population could both support the remaining and damaged axons and also remyelinate axons to promote electrical conduction (Cao Q, He Q, Wang Yet et al. Transplantation of ciliary neurotrophic factor-expressing adult oligodendrocyte precursor cells promotes remyelination and functional recovery after spinal cord injury. J. Neurosci. 2010 30(8): 2989-3001).

Oligodendrocyte progenitor cells (OPCs) are a subtype of glial cells in the central nervous system (CNS) that arise in the ventricular zones of the brain and spinal cord and migrate throughout the developing CNS before maturing into oligodendrocytes. Mature oligodendrocytes produce the myelin sheath that insulates neuronal axons and remyelinate CNS lesions where the myelin sheath has been lost. Oligodendrocytes also contribute to neuroprotection through other mechanisms, including production of neurotrophic factors that promote neuronal survival (Wilkins et al., 2001 Glia 36(1):48-57; Dai et al., 2003 J Neurosci. 23(13):5846-53; Du and Dreyfus, 2002 J Neurosci Res. 68(6):647-54). Additionally, OPCs are known to produce Decorin, a secreted factor which has been shown to suppress CNS scarring (Esmaeili, Berry et al, 2014, Gubbiotti, Vallet et al. 2016). Unlike most progenitor cells, OPCs remain abundant in the adult CNS where they retain the ability to generate new oligodendrocytes. Accordingly, OPCs and mature oligodendrocytes derived from OPCs are an important therapeutic target for demyelinating and dysmyelinating disorders (such as multiple sclerosis, adrenoleukodystrophy and adrenomyeloneuropathy), other neurodegenerative disorders (such as Alzheimer's disease, amyotrophic lateral sclerosis, and Huntington's disease) and acute neurological injuries (such as stroke and spinal cord injury (SCI)).

Propagation and Culture of Undifferentiated Pluripotent Stem Cells

In certain embodiments, the present disclosure provides methods to produce large numbers of highly pure, characterized oligodendrocyte progenitor cells from pluripotent stem cells. Derivation of oligodendrocyte progenitor cells (OPCs) from pluripotent stem cells according to the methods of the invention provides a renewable and scalable source of OPCs for a number of important therapeutic, research, development, and commercial purposes, including treatment of acute spinal cord injury.

Methods of propagation and culture of undifferentiated pluripotent stem cells have been previously described. With respect to tissue and cell culture of pluripotent stem cells, the reader may wish to refer to any of numerous publications available in the art, e.g., Teratocarcinomas and Embryonic Stem cells: A Practical Approach (E. J. Robertson, Ed., IRL Press Ltd. 1987); Guide to Techniques in Mouse Development (P. M. Wasserman et al., Eds., Academic Press 1993); Embryonic Stem Cell Differentiation in Vitro (M. V. Wiles, Meth. Enzymol. 225:900, 1993); Properties and Uses of Embryonic Stem Cells: Prospects for Application to Human Biology and Gene Therapy (P. D. Rathj en et al., Reprod. Fertil. Dev. 10:31, 1998; and R. I. Freshney, Culture of Animal Cells, Wiley-Liss, New York, 2000).

In certain embodiments, a method can be carried out on a pluripotent stem cell line. In other embodiments, a method can be carried out on an embryonic stem cell line. In an embodiment, a method can be carried out on a plurality of undifferentiated stem cells that are derived from an H1, H7, H9, H13, or H14 cell line. In another embodiment, undifferentiated stem cells can be derived from an induced pluripotent stem cell (iPS) line. In another embodiment, a method can be carried out on a primate pluripotent stem (pPS) cell line. In yet another embodiment, undifferentiated stem cells can be derived from parthenotes, which are embryos stimulated to produce hESCs without fertilization.

In one embodiment, undifferentiated pluripotent stem cells can be maintained in an undifferentiated state without added feeder cells (see, e.g., (2004) Rosler et al., Dev. Dynam. 229:259). Feeder-free cultures are typically supported by a nutrient medium containing factors that promote proliferation of the cells without differentiation (see, e.g., U.S. Pat. No. 6,800,480). In one embodiment, conditioned media containing such factors can be used. Conditioned media can be obtained by culturing the media with cells secreting such factors. Suitable cells include, but are not limited to, irradiated (4,000 Rad) primary mouse embryonic fibroblasts, telomerized mouse fibroblasts, or fibroblast-like cells derived from pPS cells (U.S. Pat. No. 6,642,048). Medium can be conditioned by plating the feeders in a serum free medium, such as knock-out DMEM supplemented with 20% serum replacement and 4 ng/mL bFGF. Medium that has been conditioned for 1-2 days can be supplemented with further bFGF, and used to support pPS cell culture for 1-2 days (see. e.g., WO 01/51616; Xu et al., (2001) Nat. Biotechnol. 19:971).

Alternatively, fresh or non-conditioned medium can be used, which has been supplemented with added factors (such as, e.g., a fibroblast growth factor or forskolin) that promote proliferation of the cells in an undifferentiated form. Non-limiting examples include a base medium like X-VIVO™ 10 (Lonza, Walkersville, Md.) or QBSF™-60 (Quality Biological Inc. Gaithersburg, Md.), supplemented with bFGF at 40-80 ng/mL, and optionally containing SCF (15 ng/mL), or Flt3 ligand (75 ng/mL) (see, e.g., Xu et al., (2005) Stem Cells 23(3):315). These media formulations have the advantage of supporting cell growth at 2-3 times the rate in other systems (see, e.g., WO 03/020920). In one embodiment, undifferentiated pluripotent cells such as hESCs, can be cultured in a media comprising bFGF and TGFP. Non-limiting example concentrations of bFGF include about 80 ng/ml. Non-limiting example concentrations of TGFP include about 0.5 ng/ml.

In one embodiment, undifferentiated pluripotent cells can be cultured on a layer of feeder cells, typically fibroblasts derived from embryonic or fetal tissue (Thomson et al. (1998) Science 282:1145). Feeder cells can be derived, inter alia, from a human or a murine source. Human feeder cells can be isolated from various human tissues, or can be derived via differentiation of human embryonic stem cells into fibroblast cells (see, e.g., WO 01/51616). In one embodiment, human feeder cells that can be used include, but are not limited to, placental fibroblasts (see, e.g., Genbacev et al. (2005) Fertil. Steril. 83(5):1517), fallopian tube epithelial cells (see, e.g., Richards et al. (2002) Nat. Biotechnol., 20:933), foreskin fibroblasts (see, e.g., Amit et al. (2003) Biol. Reprod.68:2150), and uterine endometrial cells (see, e.g., Lee et al. (2005) Biol. Reprod. 72(1):42).

Various solid surfaces can be used in the culturing of undifferentiated pluripotent cells. Those solid surfaces include, but are not limited to, standard commercially available cell culture plates, such as 6-well, 24-well, 96-well, or 144-well plates. Other solid surfaces include, but are not limited to, microcarriers and disks. Solid surfaces suitable for growing undifferentiated pluripotent cells can be made of a variety of substances including, but not limited to, glass or plastic such as polystyrene, polyvinylchloride, polycarbonate, polytetrafluorethylene, melinex, thermanox, or combinations thereof. In one embodiment, suitable surfaces can comprise one or more polymers, such as, e.g., one or more acrylates. In one embodiment, a solid surface can be three-dimensional in shape. Non-limiting examples of three-dimensional solid surfaces are described, e.g., in U.S. Patent Pub. No. 2005/0031598.

In one embodiment, undifferentiated stem cells can be grown under feeder-free conditions on a growth substrate. In one embodiment, a growth substrate can be Matrigel® (e.g., Matrigel® or Matrigel® GFR), recombinant Laminin, or Vitronectin. In another embodiment, undifferentiated stem cells can be subcultured using various methods such as using collagenase, or such as manual scraping. In another embodiment, undifferentiated stem cells can be subcultured using non-enzymatic means, such as 0.5 mM EDTA in PBS, or such as using ReLeSR™. In an embodiment, a plurality of undifferentiated stem cells are seeded or subcultured at a seeding density that allows the cells to reach confluence in about three to about ten days. In an embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

Any of a variety of suitable cell culture and sub-culturing techniques can be used to culture cells in accordance with the present disclosure. For example, in one embodiment, a culture medium can be exchanged at a suitable time interval. In one embodiment, a culture medium can be completely exchanged daily, initiating about 2 days after sub-culturing of the cells. In another embodiment, when a culture reaches about 90% colony coverage, a surrogate flask can be sacrificed and enumerated using one or more suitable reagents, such as, e.g., Collagenase IV and 0.05% Trypsin-EDTA in series to achieve a single cell suspension for quantification. In an embodiment, a plurality undifferentiated stem cells can then be subcultured before seeding the cells on a suitable growth substrate (e.g., Matrigel® GFR) at a seeding density that allows the cells to reach confluence over a suitable period of time, such as, e.g., in about three to ten days. In one embodiment, undifferentiated stem cells can be subcultured using Collagenase IV and expanded on a recombinant laminin matrix. In one embodiment, undifferentiated stem cells can be subcultured using Collagenase IV and expanded on a Matrigel® matrix. In one embodiment, undifferentiated stem cells can be subcultured using ReLeSR™ and expanded on a Vitronectin matrix.

In one embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment, the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

Oligodendrocyte Progenitor Cell Compositions

As discussed above, the present disclosure provides compositions comprising a population of oligodendrocyte progenitor cells (OPCs) as well as methods of making and using the same from use in the treatment of acute spinal cord injury and other related CNS conditions. In certain embodiments, the OPCs of the present disclosure are capable of producing and secreting one or more biological factors that may augment neural repair.

In one embodiment, a cell population can have a common genetic background. In an embodiment, a cell population may be derived from one host. In an embodiment, a cell population can be derived from a pluripotent stem cell line. In another embodiment, a cell population can be derived from an embryonic stem cell line. In an embodiment, a cell population can be derived from a hESC line. In an embodiment, a hESC line can be an H1, H7, H9, H13, or H14 cell line. In another embodiment, a cell population can be derived from an induced pluripotent stem cell (iPS) line. In an embodiment a cell population can be derived from a subject in need thereof (e.g., a cell population can be derived from a subject that is in need to treatment). In yet another embodiment, a hESC line can be derived from parthenotes, which are embryos stimulated to produce hESCs without fertilization.

In certain embodiments, the OPCs of the present disclosure express one or more markers chosen from Nestin, NG2, Olig 1 and PDGF-Ra. In certain embodiments, the OPCs of the present disclosure express all of the markers Nestin, NG2, Olig 1 and PDGF-Ra.

In certain embodiments, the OPCs of the present disclosure are capable of secreting one or more biological factors. In certain embodiments, the one or more biological factors secreted by the OPCs of the present disclosure may promote, without limitation, neural repair, axonal outgrowth and/or glial differentiation, or any combination thereof. In some embodiments, the OPCs are capable of secreting one or more factors that stimulate axonal outgrowth. In some embodiments, the OPCs are capable of secreting one or more factors promoting glial differentiation by neural precursor cells. In some embodiments, the OPCs are capable of secreting one or more chemoattractants for neural precursor cells. In some embodiments, the OPCs are capable of secreting one or more inhibitors of matrix metalloproteinases. In some embodiments, the OPCs are capable of secreting one or more factors inhibiting cell death after spinal cord injury. In some embodiments, the OPCs are capable of secreting one or more factors that are upregulated post-cellular injury and that aid in the clearance of misfolded proteins.

In certain embodiments, the OPCs are capable of producing and secreting one or more biological factors selected from MCP-1, Clusterin, ApoE, TIMP1 and TIMP2. In further embodiments the OPCs are capable of producing and secreting MCP-1 and one or more of the factors selected from Clusterin, ApoE, TIMP1 and TIMP2. In yet further embodiments, the OPCs are capable of producing and secreting all of the factors MCP-1, Clusterin, ApoE, TIMP1 and TIMP2.

In an embodiment, a biological factor can be secreted by a composition comprising a population of OPCs at a concentration of more than about 50 pg/ml, such as more than about 100 pg/ml, such as more than about 200 pg/ml, such as more than about 300 pg/ml, such as more than about 400 pg/ml, such as more than about 500 pg/ml, such as more than about 1,000 pg/ml, such as more than about 2,000 pg/ml, such as more than about 3,000 pg/ml, such as more than about 4,000 pg/ml, such as more than about 5,000 pg/ml, such as more than about 6,000 pg/ml, or such as more than about 7,000 pg/ml. In certain embodiments, a biological factor can be secreted by a composition comprising a population of cells comprising OPCs at a concentration ranging from about 50 pg/ml to about 100,000 pg/ml, such as about 100 pg/ml, such as about 150 pg/ml, such as about 200 pg/ml, such as about 250 pg/ml, such as about 300 pg/ml, such as about 350 pg/ml, such as about 400 pg/ml, such as about 450 pg/ml, such as about 500 pg/ml, such as about 550 pg/ml, such as about 600 pg/ml, such as about 650 pg/ml, such as about 700 pg/ml, such as about 750 pg/ml, such as about 800 pg/ml, such as about 850 pg/ml, such as about 900 pg/ml, such as about 1,000 pg/ml, such as about 1,500 pg/ml, such as about 2,000 pg/ml, such as about 2,500 pg/ml, such as about 3,000 pg/ml, such as about 3,500 pg/ml, such as about 4,000 pg/ml, such as about 4,500 pg/ml, such as about 5,000 pg/ml, such as about 5,500 pg/ml, such as about 6,000 pg/ml, such as about 6,500 pg/ml, such as about 7,000 pg/ml, such as about 7,500 pg/ml, such as about 8,000 pg/ml, such as about 8,500 pg/ml, such as about 9,000 pg/ml, such as about 10,000 pg/ml, such as about 15,000 pg/ml, such as about 20,000 pg/ml, such as about 25,000 pg/ml, such as about 30,000 pg/ml, such as about 35,000 pg/ml, such as about 40,000 pg/ml, such as about 45,000 pg/ml, such as about 50,000 pg/ml, such as about 55,000 pg/ml, such as about 60,000 pg/ml, such as about 65,000 pg/ml, such as about 70,000 pg/ml, such as about 75,000 pg/ml, such as about 80,000 pg/ml, such as about 85,000 pg/ml, such as about 90,000 pg/ml, such as about 95,000 pg/ml.

In certain embodiments, a biological factor can be secreted by a composition comprising a population of cells comprising OPCs at a concentration ranging from about 1,000 pg/ml to about 10,000 pg/ml, such as about 1,000 pg/ml to about 2,000 pg/ml, such as about 2,000 pg/ml to about 3,000 pg/ml, such as about 3,000 pg/ml to about 4,000 pg/ml, such as about 4,000 pg/ml to about 5,000 pg/ml, such as about 5,000 pg/ml to about 6,000 pg/ml, such as about 6,000 pg/ml to about 7,000 pg/ml, such as about 7,000 pg/ml to about 8,000 pg/ml, such as about 8,000 pg/ml to about 9,000 pg/ml, or such as about 9,000 pg/ml to about 10,000 pg/ml.

In certain embodiments, a biological factor can be secreted by a composition comprising a population of cells comprising OPCs at a concentration ranging from about 10,000 pg/ml to about 100,000 pg/ml, such as about 10,000 pg/ml to about 20,000 pg/ml, such as about 20,000 pg/ml to about 30,000 pg/ml, such as about 30,000 pg/ml to about 40,000 pg/ml, such as about 40,000 pg/ml to about 50,000 pg/ml, such as about 50,000 pg/ml to about 60,000 pg/ml, such as about 60,000 pg/ml to about 70,000 pg/ml, such as about 70,000 pg/ml to about 80,000 pg/ml, such as about 80,000 pg/ml to about 90,000 pg/ml, or such as about 90,000 pg/ml to about 100,000 pg/ml.

In some embodiments, Clusterin can be secreted by a composition comprising a population of cells comprising OPCs at a concentration ranging from about 1,000 pg/ml to about 100,000 pg/ml. In certain embodiments, Clusterin can be secreted by a composition comprising a population of cells comprising OPCs at a concentration ranging from about 10,000 pg/ml to about 50,000 pg/ml. In some embodiments, MCP-1 can be secreted by a composition comprising a population of cells comprising OPCs at a concentration ranging from about 500 pg/ml to about 50,000 pg/ml. In certain embodiments, MCP-1 can be secreted by a composition comprising a population of cells comprising OPCs at a concentration ranging from about 5,000 pg/ml to about 15,000 pg/ml. In some embodiments, ApoE can be secreted by a composition comprising a population of cells comprising OPCs at a concentration ranging from about 100 pg/ml to about 10,000 pg/ml. In certain embodiments, ApoE can be secreted by a composition comprising a population of cells comprising OPCs at a concentration ranging from about 500 pg/ml to about 5,000 pg/ml. In some embodiments, TIMP1 can be secreted by a composition comprising a population of cells comprising OPCs at a concentration ranging from about 100 pg/ml to about 10,000 pg/ml. In certain embodiments, TIMP1 can be secreted by a composition comprising a population of cells comprising OPCs at a concentration ranging from about 500 pg/ml to about 5,000 pg/ml. In some embodiments, TIMP2 can be secreted by a composition comprising a population of cells comprising OPCs at a concentration ranging from about 100 pg/ml to about 10,000 pg/ml. In certain embodiments, TIMP2 can be secreted by a composition comprising a population of cells comprising OPCs at a concentration ranging from about 500 pg/ml to about 5,000 pg/ml.

Pharmaceutical Compositions

The OPCs of the present disclosure can be administered to a subject in need of therapy, such as SCI therapy. Alternatively, the cells of the present disclosure can be administered to the subject in need of SCI therapy in a pharmaceutical composition together with a suitable carrier and/or using a delivery system.

As used herein, the term “pharmaceutical composition” refers to a preparation comprising a therapeutic agent or therapeutic agents in combination with other components, such as physiologically suitable carriers and excipients.

As used herein, the term “therapeutic agent” can refer to the cells of the present disclosure accountable for a biological effect in the subject. Depending on the embodiment of the disclosure, “therapeutic agent” can refer to the oligodendrocyte progenitor cells of the disclosure. Alternatively, “therapeutic agent” can refer to one or more factors secreted by the oligodendrocyte progenitor cells of the disclosure.

As used herein, the terms “carrier”, “pharmaceutically acceptable carrier” and “biologically acceptable carrier” may be used interchangeably and refer to a diluent or a carrier substance that does not cause significant adverse effects or irritation in the subject and does not abrogate the biological activity or effect of the therapeutic agent. In certain embodiments, a pharmaceutically acceptable carrier can comprise dimethyl sulfoxide (DMSO). In other embodiments, a pharmaceutically acceptable carrier does not comprise dimethyl sulfoxide. The term “excipient” refers to an inert substance added to a pharmaceutical composition to further facilitate administration of the therapeutic agent.

The therapeutic agent or agents of the present disclosure can be administered as a component of a hydrogel, such as those described in U.S. patent application Ser. No. 14/275,795, filed May 12, 2014, and U.S. Pat. Nos. 8,324,184 and 7,928,069.

The compositions in accordance with the present disclosure can be formulated for parenteral administration by injection, e.g., by bolus injection or continuous infusion. Formulations for injection can be presented in unit dosage form, e.g., in ampoules or in multi-dose containers, with an added preservative. The compositions can contain formulatory agents such as suspending, stabilizing and/or dispersing agents. In certain embodiments, the compositions can be formulated to be adapted for cryopreservation.

The compositions in accordance with the present disclosure can be formulated for administration via injection to the spinal cord of a subject. The compositions may also be formulation for direct injection to the spinal cord of a subject. The compositions can be formulated for administration via implantation or other delivery methods. In certain embodiments, a composition in accordance with the present disclosure can be formulated for intracerebral, intraventricular, intrathecal, intranasal, or intracisternal administration to a subject. In certain embodiments, a composition in accordance with the present disclosure can be formulated for administration via an injection directly into or immediately adjacent to an infarct cavity in the brain of a subject. In certain embodiments, a composition in accordance with the present disclosure can be formulated for administration through implantation. In certain embodiments, a composition in accordance with the present disclosure can be formulated for administration through other suitable delivery methods. In certain embodiments, a composition in accordance with the present disclosure can be formulated as a solution.

In certain embodiments, a composition in accordance with the present disclosure can comprise from about 1×10⁶ to about 5×10⁸ cells per milliliter, such as about 1×10⁶ cells per milliliter, such as about 2×10⁶ cells per milliliter, such as about 3×10⁶ cells per milliliter, such as about 4×10⁶ cells per milliliter, such as about 5×10⁶ cells per milliliter, such as about 6×10⁶ cells per milliliter, such as about 7×10⁶ cells per milliliter, such as about 8×10⁶ cells per milliliter, such as about 9×10⁶ cells per milliliter, such as about 1×10⁷ cells per milliliter, such as about 2×10⁷ cells per milliliter, such as about 3×10⁷ cells per milliliter, such as about 4×10⁷ cells per milliliter, such as about 5×10⁷ cells per milliliter, such as about 6×10⁷ cells per milliliter, such as about 7×10⁷ cells per milliliter, such as about 8×10⁷ cells per milliliter, such as about 9×10⁷ cells per milliliter, such as about 1×10⁸ cells per milliliter, such as about 2×10⁸ cells per milliliter, such as about 3×10⁸ cells per milliliter, such as about 4×10⁸ cells per milliliter, or such as about 5×10⁸ cells per milliliter. In certain embodiments, a composition in accordance with the present disclosure can comprise from about 1×10⁸ to about 5×10⁸ cells per milliliter, such as about 1×10⁸ to about 4×10⁸ cells per milliliter, such as about 2×10⁸ to about 5×10⁸ cells per milliliter, such as about 1×10⁸ to about 3×10⁸ cells per milliliter, such as about 2×10⁸ to about 4×10⁸ cells per milliliter, or such as about 3×10⁸ to about 5×10⁸ cells per milliliter. In yet another embodiment, a composition in accordance with the present disclosure can comprise from about 1×10⁷ to about 1×10⁸ cells per milliliter, such as about 2×10⁷ to about 9×10⁷ cells per milliliter, such as about 3×10⁷ to about 8×10⁷ cells per milliliter, such as about 4×10⁷ to about 7×10⁷ cells per milliliter, or such as about 5×10⁷ to about 6×10⁷ cells per milliliter. In an embodiment, a composition in accordance with the present disclosure can comprise from about 1×10⁶ to about 1×10⁷ cells per milliliter, such as about 2×10⁶ to about 9×10⁶ cells per milliliter, such as about 3×10⁶ to about 8×10⁶ cells per milliliter, such as about 4×10⁶ to about 7×10⁶ cells per milliliter, or such as about 5×10⁶ to about 6×10⁶ cells per milliliter. In yet another embodiment, a composition in accordance with the present disclosure can comprise at least about 1×10⁶ cells per milliliter, such as at least about 2×10⁶ cells per milliliter, such as at least about 3×10⁶ cells per milliliter, such as at least about 4×10⁶ cells per milliliter, such as at least about 5×10⁶ cells per milliliter, such as at least about 6×10⁶ cells per milliliter, such as at least about 7×10⁶ cells per milliliter, such as at least about 8×10⁶ cells per milliliter, such as at least about 9×10⁶ cells per milliliter, such as at least about 1×10⁷ cells per milliliter, such as at least about 2×10⁷ cells per milliliter, such as at least about 3×10⁷ cells per milliliter, such as at least about 4×10⁷ cells per milliliter, or such as at least about 5×10⁷ cells per milliliter. In an embodiment, a composition in accordance with the present disclosure can comprise up to about 1×10⁸ cells or more, such as up to about 2×10⁸ cells per milliliter or more, such as up to about 3×10⁸ cells per milliliter or more, such as up to about 4×10⁸ cells per milliliter or more, such as up to about 5×10⁸ cells per milliliter or more, or such as up to about 6×10⁸ cells per milliliter.

In an embodiment, a composition in accordance with the present disclosure can comprise from about 4×10⁷ to about 2×10⁸ cells per milliliter.

In yet another embodiment, a composition in accordance with the present disclosure can have a volume ranging from about 10 microliters to about 5 milliliters, such as about 20 microliters, such as about 30 microliters, such as about 40 microliters, such as about 50 microliters, such as about 60 microliters, such as about 70 microliters, such as about 80 microliters, such as about 90 microliters, such as about 100 microliters, such as about 200 microliters, such as about 300 microliters, such as about 400 microliters, such as about 500 microliters, such as about 600 microliters, such as about 700 microliters, such as about 800 microliters, such as about 900 microliters, such as about 1 milliliter, such as about 1.5 milliliters, such as about 2 milliliters, such as about 2.5 milliliters, such as about 3 milliliters, such as about 3.5 milliliters, such as about 4 milliliters, or such as about 4.5 milliliters. In an embodiment, a composition in accordance with the present disclosure can have a volume ranging from about 10 microliters to about 100 microliters, such as about 20 microliters to about 90 microliters, such as about 30 microliters to about 80 microliters, such as about 40 microliters to about 70 microliters, or such as about 50 microliters to about 60 microliters. In another embodiment, a composition in accordance with the present disclosure can have a volume ranging from about 100 microliters to about 1 milliliter, such as about 200 microliters to about 900 microliters, such as about 300 microliters to about 800 microliters, such as about 400 microliters to about 700 microliters, or such as about 500 microliters to about 600 microliters. In yet another embodiment, a composition in accordance with the present disclosure can have a volume ranging from about 1 milliliter to about 5 milliliters, such as about 2 milliliter to about 5 milliliters, such as about 1 milliliter to about 4 milliliters, such as about 1 milliliter to about 3 milliliters, such as about 2 milliliter to about 4 milliliters, or such as about 3 milliliter to about 5 milliliters. In an embodiment, a composition in accordance with the present disclosure can have a volume of about 20 microliters to about 500 microliters. In another embodiment, a composition in accordance with the present disclosure can have a volume of about 50 microliters to about 100 microliters. In yet another embodiment, a composition in accordance with the present disclosure can have a volume of about 50 microliters to about 200 microliters. In another embodiment, a composition in accordance with the present disclosure can have a volume of about 20 microliters to about 400 microliters.

In certain embodiments, the present disclosure provides a container comprising a composition comprising a population of OPCs derived in accordance with one or more methods of the present disclosure. In certain embodiments, a container can be configured for cryopreservation. In certain embodiments, a container can be configured for administration to a subject in need thereof. In certain embodiments, a container can be a prefilled syringe.

For general principles in medicinal formulation, the reader is referred to Allogeneic Stem Cell Transplantation, Lazarus and Laughlin Eds. Springer Science+Business Media LLC 2010; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. In certain embodiments, the composition can also comprise or be accompanied by one or more other ingredients that facilitate the engraftment or functional mobilization of the enriched target cells. Suitable ingredients can include matrix proteins that support or promote adhesion of the target cell type or that promote vascularization of the implanted tissue.

Uses of the Cells of the Present Disclosure

In various embodiments as described herein, the present disclosure provides methods of using a cell population that comprises pluripotent stem cell-derived OPCs for improving one or more neurological functions in a subject in need of therapy. In certain embodiments, methods for using pluripotent stem-cell derived OPCs in the treatment of acute spinal cord injury are provided. In other embodiments, methods for using pluripotent stem-cell derived OPCs in the treatment of other traumatic CNS injuries are provided. In other embodiments, methods for using pluripotent stem-cell derived OPCs in the treatment of non-traumatic CNS disorders or conditions are provided. In certain embodiments, a cell population in accordance with the present disclosure can be injected or implanted into a subject in need thereof.

In certain embodiments, methods for using pluripotent stem-cell derived OPCs in the treatment of conditions requiring myelin repair or remyelination are provided. The following are non-limiting examples of conditions, diseases and pathologies requiring myelin repair or remyelination: multiple sclerosis, the leukodystrophies, the Guillain-Barre Syndrome, the Charcot-Marie-Tooth neuropathy, Tay-Sachs disease, Niemann-Pick disease, Gaucher disease and Hurler syndrome. Other conditions that result in demyelination include but are not limited to inflammation, stroke, immune disorders, metabolic disorders and nutritional deficiencies (such as lack of vitamin B12). The OPCs of the present disclosure can also be used for myelin repair or remyelination in traumatic injuries resulting in loss of myelination, such as acute spinal cord injury.

The OPCs are administered in a manner that permits them to graft or migrate to the intended tissue site and reconstitute or regenerate the functionally deficient area. Administration of the cells can be achieved by any method known in the art. For example the cells can be administered surgically directly to the organ or tissue in need of a cellular transplant. Alternatively non-invasive procedures can be used to administer the cells to the subject. Non-limiting examples of non-invasive delivery methods include the use of syringes and/or catheters to deliver the cells into the organ or tissue in need of cellular therapy.

The subject receiving the OPCs of the present disclosure may be treated to reduce immune rejection of the transplanted cells. Methods contemplated include the administration of traditional immunosuppressive drugs such as, e.g., tacrolimus, cyclosporin A (Dunn et al., Drugs 61:1957, 2001), or inducing immunotolerance using a matched population of pluripotent stem cell-derived cells (WO 02/44343; U.S. Pat. No. 6,280,718; WO 03/050251). Alternatively a combination of anti-inflammatory (such as prednisone) and immunosuppressive drugs can be used. The OPCs of the invention can be supplied in the form of a pharmaceutical composition, comprising an isotonic excipient prepared under sufficiently sterile conditions for human administration.

Use in treatment of CNS traumatic injury. In certain embodiments, a cell population in accordance with the present disclosure can be capable of engrafting at a spinal cord injury site following implantation of a composition comprising the cell population into the spinal cord injury site.

In certain embodiments, a cell population in accordance with the present disclosure is capable of remaining within the spinal cord injury site of the subject for a period of about 180 days or longer following implantation of a dose of the composition into the spinal cord injury site. In other embodiments, a cell population in accordance with the present disclosure is capable of remaining within the spinal cord injury site of the subject for a period of about 2 years or longer following implantation of a dose of the composition into the spinal cord injury site. In further embodiments, a cell population in accordance with the present disclosure is capable of remaining within the spinal cord injury site of the subject for a period of about 3 years or longer following implantation of a dose of the composition into the spinal cord injury site. In yet further embodiments, a cell population in accordance with the present disclosure is capable of remaining within the spinal cord injury site of the subject for a period of about 4 years or longer following implantation of a dose of the composition into the spinal cord injury site.

In certain embodiments, a cell composition in accordance with the present disclosure is capable of reducing spinal cord injury-induced parenchymal cavitation in a subject. In certain embodiments, a lesion volume is reduced by formation of a tissue matrix in the spinal cord injury site. In certain embodiments, the cells of the present disclosure are capable of forming a tissue matrix in the spinal cord injury site within about 180 days or less. In certain embodiments, the subject with reduced injury-induced parenchymal cavitation is human.

In certain embodiments, a cell population in accordance with the present disclosure can be capable of reducing a volume of an injury-induced central nervous system parenchymal cavitation in about 12 months or less. In certain embodiments, a cell population in accordance with the present disclosure can be capable of reducing a volume of an injury-induced central nervous system parenchymal cavitation in a subject in about 6 months or less, about 5 months or less, or less than about 4 months. In certain embodiments, the subject is human.

In an embodiment, one or more cells from a cell population in accordance with the present disclosure can be capable of migrating from a first location to one or more second locations within the central nervous system of a subject in need thereof. In an embodiment, one or more cells from a cell population in accordance with the present disclosure can be capable of migrating from the spinal cord of a subject to an affected tissue within the brain of the subject. In one embodiment, one or more cells from a cell population in accordance with the present disclosure can be capable of migrating from a first location within the spinal cord of a subject to a second location at an affected tissue within the spinal cord of the subject. In one embodiment, one or more cells from a cell population in accordance with the present disclosure can be capable of migrating from a first location within the brain of a subject to a second location at an affected tissue within the brain of the subject. In one embodiment, one or more cells from a cell population in accordance with the present disclosure can be capable of migrating from a first location within the brain of a subject to an affected tissue within the spinal cord of the subject. In one embodiment, one or more cells from a cell population in accordance with the present disclosure can be capable of migrating from a first location within the spinal cord of a subject to a second location at an affected tissue within the spinal cord of the subject, as well as to one or more locations at one or more affected tissues within the brain of the subject. In one embodiment, one or more cells from a cell population in accordance with the present disclosure can be capable of migrating from a first location within the brain of a subject to a second location at an affected tissue within the brain of the subject, as well as to one or more locations at one or more affected tissues within the spinal cord of the subject.

In an embodiment, one or more cells from a cell population in accordance with the present disclosure can be capable of migrating from a first location to one or more second locations at one or more affected tissues within the central nervous system of a subject in less than about 150 days, such as less than about 100 days, such as less than about 50 days, or such as less than about 10 days. In an embodiment, one or more cells from a cell population in accordance with the present disclosure can be capable of migrating from a first location to one or more second locations at one or more affected tissues within the central nervous system of a subject in about 180 days or less.

### EXAMPLES

Examples 1-8 describe the first-in-human Phase 1 safety clinical trial of oligodendrocyte progenitor cells derived from human pluripotent stem cells (LCTOPC1) which have mechanistic properties to support survival and potential repair of key cellular components and architecture of the SCI site. Example 9 describes a Phase 1/2a dose escalation study of oligodendrocyte progenitor cells derived from human pluripotent stem cells (AST-OPC1) for use in subacute cervical SCI.

Example 1—Patients and Methods

Study design. The trial design was an open-label, multicenter study. A single dose of 2×10\hat{ }6 LCTOPC1 was injected within 7 to 14 days following SCI. Subjects who received LCTOPC1 also received tacrolimus to prevent rejection. Subjects will be followed by protocol for 15 years following administration of LCTOPC1.

Study Participants. Male or female participants from 18 to 65 years of age with acute traumatic spinal cord injury were eligible for study participation. As this was a first in man study, with a risk of neurological deterioration, inclusion was limited to neurologically complete injuries (American Spinal Injury Association Impairment Scale A), with a single neurological level of injury (NLI) from levels T3-T10, with no spared motor function <5 levels (i.e. zone of partial preservation) below the single neurological level. These inclusion criteria were chosen to minimize loss of function if neurological deterioration were to occur.

Post-stabilization magnetic resonance imaging (MRI) was used to confirm the presence of a single spinal cord lesion with sufficient visualization of the spinal cord for 30 mm above and below the injury epicenter to enable post-injection safety monitoring. Participants had to be eligible for an elective surgical procedure to inject LCTOPC1 7 to 14 days following SCI.

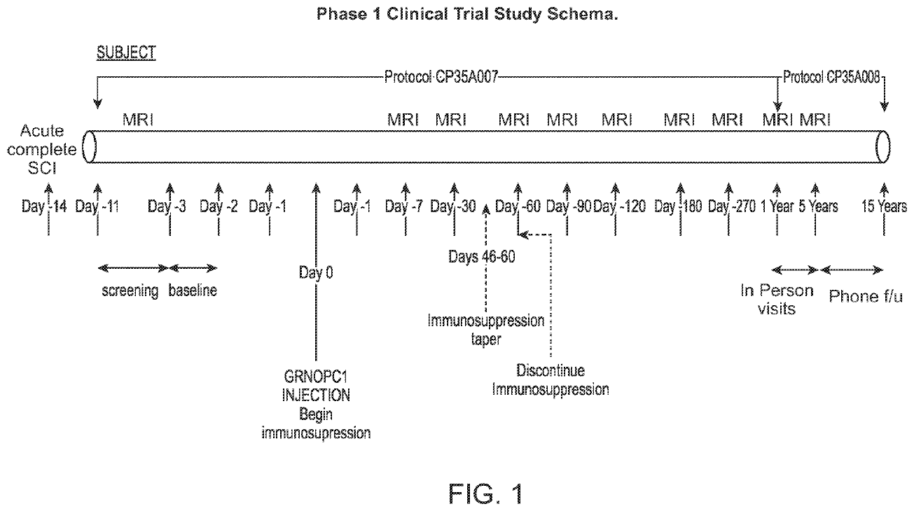

This study was a Phase 1, multi-center, non-randomized, a single group assignment interventional clinical trial. The Participants were enrolled from one of seven centers in the United States. The study was registered (NCT01217008) and the primary endpoint was safety, as measured by the frequency and severity of adverse events related to LCTOPC1, the injection procedure used to administer LCTOPC1, and/or the concomitant immunosuppression administered. The secondary endpoint was neurological function as measured by sensory scores and lower extremity motor scores on ISNCSCI examinations. The eligibility criteria are summarized in Supplemental Table 1. Participants have been followed by protocol for a total of 5 years of in-person visits and are being followed for an additional 10 years of annual phone visits. FIG. 1 provides an overall study schema for the clinical trial.

The LCTOPC1 product is a cell population containing a mixture of oligodendrocyte progenitor cells and other characterized cell types obtained following directed differentiation of undifferentiated human embryonic stem cells. The initial characterization of the LCTOPC1 population was reported by Nistor et al 2005, who showed that these cells could differentiate into oligodendroglial progenitors. Subsequent studies demonstrated that the oligodendroglial progenitor cells survived after delivery to the spinal cord injury site in an acute incomplete rat contusion injury model. The cells led to sparing of tissue at the contusion site with evidence of remyelination of denuded axons. When delivered in the acute injury period, the cells led to improvement in locomotor function as measured in standardized behavioral testing. Preclinical studies in rats and mice demonstrated that the intended clinical, cryopreserved human equivalent dose formulation of LCTOPC1 could survive and migrate after injection in the SCI site, produce neurotrophic factors to support cell survival, provide remyelination potential to support denuded axons, and lead to tissue sparing at the SCI contusion site. Moreover, studies demonstrated that the cells did not produce teratomas, and did not lead to increased pain in injured animals.

This Phase 1 clinical trial was reviewed by the FDA, the Data and Safety Monitoring Board (DSMB), the SCI clinical community, surgical and outcomes steering committees, internal and external ethics committees, internal and clinical trial site stem cell research oversight committees, and the IRBs for each participating clinical trial site. As a first-in-human study, the trial design accounted for the need to minimize the risk to participants, and hence individuals with complete SCI localized between the thoracic neurological levels T3-T11 were chosen for intervention. The trial was an open-label, unblinded, non-randomized, non-placebo-controlled study to establish the safety of intraparenchymal injection of LCTPOC1 as well as to determine changes in neurological function.

Determining the long-term safety of stem cell therapeutics is a critical step in enabling future trials to investigate novel stem cell therapeutics or combination therapies. Ten years post-implantation, there have been no medical or neurological complications to indicate that LCTOPC1 implantation is unsafe. Specifically, there have been no Serious Adverse Events (SAEs) related to the procedure, cell implant, or immunosuppression. In addition, there have been no significant changes in neurological function. Safety data from this first-in-human study supported progression to a clinical trial for individuals with cervical spinal cord injuries.

The starting material for the production of AST-OPC1 is an H1 master cell bank produced from the H1 uhESC line derived at the University of Wisconsin in 1998. Compositional analysis of LCTOPC1 by immunocytochemistry and flow cytometry indicates that the cell population is comprised mostly of neural lineage cells of the oligodendrocyte progenitor phenotype. In this safety study, the intended route of administration for LCTOPC1 was a direct injection of 2×10\hat{ }6 viable LCTOPC1 cells into the spinal cord at a level 5 mm caudal to the injury epicenter.

The rationale for the selection of this dose was based upon pre-clinical studies involving rats and mice, and upon dose extrapolation to humans using two methods: 1) comparing the relative sizes of the human and rat spinal cords, and 2) evaluating tumorigenicity with respect to the absolute number of injected cells. Further, the rationale for the selection of this route of delivery was based on the results from nonclinical pharmacology studies, which showed that LCTOPC1 has properties that support repair of pathology in spinal cord lesions. Results from these studies also demonstrated that improvements in locomotor recovery were associated with robust LCTOPC1 cell survival at the lesion site.

Spinal cord injuries localized between the T3 and T11 neurological level, as assessed by the ISNCSCI, were chosen as the target for intervention. The primary goal of this first-in-human study was to establish the safety of intraparenchymal injection of hESC derived oligodendrocyte precursor cells into the spinal cord of individuals between 7-14 days post-injury. The secondary outcome measure in this trial was the ISNCSCI examination which allowed for the identification of motor and sensory changes at any of 13 in-person evaluations scheduled within the first five years post-injection.

Ten-years post-implantation, there have been no medical or neurological complications to indicate that the cell implantation was unsafe. Specifically, there have been no serious adverse events (SAEs) related to the procedure, cell implant, or immunosuppression. This report will review the first 10 years of data from this landmark clinical trial including early post-operative events, in-person follow-up through year 5 and conclude with data from telephone follow-up to the current time.

Investigational Product, Dose Preparation, Dose and Mode of Administration. LCTOPC1 is a cell population containing a mixture of oligodendrocyte progenitor cells and other characterized cell types that are obtained following differentiation of undifferentiated human embryonic stem cells (hESC) from the H1 stem cell line, produced at the University of Wisconsin in 1998.

Compositional analysis of LCTOPC1 by immunocytochemistry and flow cytometry indicates that the cell population is comprised mostly of neural lineage cells of the oligodendrocyte progenitor phenotype. In this safety study, the intended route of administration for LCTOPC1 was a direct injection of 2×10\hat{ }6 viable LCTOPC1 cells into the spinal cord at a level 5 mm caudal to the injury epicenter.

LCTOPC1 is a cryopreserved cell therapy product. At the time of cryopreservation, each vial contained 7.5×10⁶ viable cells in 1.2 mL of cryoprotectant solution. LCTOPC1 was supplied in 2.0 mL cryovials and shipped to the clinical sites in the vapor phase of liquid nitrogen and stored under the same conditions at the site. Prior to administration, LCTOPC1 was thawed and prepared by study personnel who were trained and qualified in the preparation of the study drug.

Participants received a single administration of 2×10⁶ viable LCTOPC1 cells suspended in Hank's balanced salt solution (HBSS) total volume per dose=50 μL. The rationale for selection of this dose was based upon pre-clinical studies involving rats and mice, and upon dose extrapolation to humans using two methods: 1) comparing the relative sizes of the human and rat spinal cords, and 2) evaluating tumorigenicity risks with respect to the absolute number of injected cells. At that time during the development of LCTOPC1, 2×10⁶ cells was the highest dose that was feasible to administer in the injured rat spinal cord and the rat was the largest animal that could be utilized to satisfy the animal number required for the IND-enabling studies for this novel product. Hence, to be conservative, 2×10⁶ cells, the highest dose tested in rats, was used as the dose for the Phase 1 trial. Participants who received LCTOPC1 also received tacrolimus to prevent rejection of this allogeneic cell-based product.

The intended route of administration for LCTOPC1 was intra-parenchymal at a depth of 6 mm, in the midline, 5 mm caudal to the epicenter of injury as determined by MRI, as modeled in preclinical studies. A caudal injection was selected out of an abundance of caution to avoid any potential direct tissue damage above the injury level. Based on preclinical studies, it was anticipated that the injected cells would migrate rostrally throughout the injury site. LCTOPC1 was administered to the spinal cord in a dedicated surgical procedure 7-14 days following injury. This time frame was chosen based on results of animal studies suggesting poor graft survival for implantation within the first 7 days of injury while attempting to maximize the potential neuroprotective and remyelinating effect. A custom-designed syringe positioning device was utilized to assist neurosurgeons with the controlled delivery of the cells.

Tacrolimus Immunosuppression. Immunosuppression with tacrolimus was initiated between 6 and 12 hours after injection of LCTOPC1. If the participant was unable to take oral medication, tacrolimus was administered intravenously at a starting dose of 0.01 mg/kg/day, given as a continuous intravenous infusion. Participants were switched to oral tacrolimus as possible. The starting dose for oral tacrolimus was 0.03 mg/kg/day, divided into 2 daily doses. The tacrolimus dose was adjusted to achieve a target whole blood trough level of 3 to 7 ng/mL.

On Day 46, the tacrolimus dose was decreased by 50% (rounded to the nearest 0.5 mg, as this was the smallest capsule size available). On Day 53, the tacrolimus dose was decreased by another 50% (rounded to the nearest 0.5 mg). If the rounded total daily dose was 0.5 mg or lower, the participant received 0.5 mg once per day until tacrolimus was discontinued. Tacrolimus was discontinued at Day 60. The dose of tacrolimus was lowered if the trough blood level exceeded 7 ng/mL. In addition, an expert reviewed all ISNCSCI examination forms to assess whether there were any changes in neurological function that may have been associated with tacrolimus tapering and/or discontinuation.

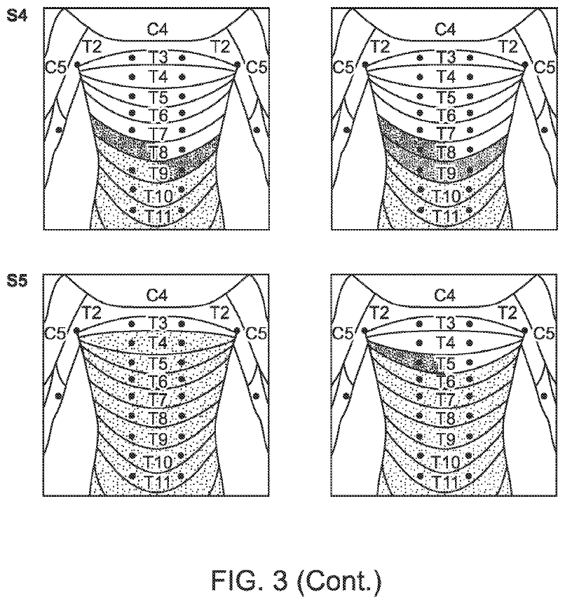

Follow-up and Assessments. An overview of study visits for the one-year protocol follow-up (CP35A007) and 2-15-year protocol follow-up (CP35A008) is provided in the study schema (FIG. 1). As this was a first clinical trial of cells derived from hESCs, a high number of study visits and long-term follow-up were required. In the one-year protocol, three study visits were required prior to product administration, with 13 in the first year following study administration. For the long-term protocol, annual visits were required in years 2-5. Subsequent to the year 5 annual visit, follow-up was by annual phone questionnaires (FIG. 4) and in-person evaluations, as necessary. Phone assessments include documentation of all new medications taken for longer than 30 days, admissions to the hospital, and documentation of AEs and SAEs.

Safety Assessments. The primary endpoint of the Phase 1 clinical trial was safety, as measured by the frequency and severity of adverse events (AEs) within 1 year of LCTOPC1 injection that were related to LCTOPC1, the injection procedure used to administer LCTOPC1, and/or the concomitant immunosuppression administered. Safety assessments included physical examination, vital signs, ISNCSCI neurological examination, pain questionnaire, electrocardiogram, MRI, laboratory tests for hematology and blood chemistry, laboratory tests for immunosuppression safety monitoring (whole blood trough levels of tacrolimus, serum levels of creatinine, potassium, magnesium, phosphate, ionized calcium, aspartate aminotransferase, alanine aminotransferase, and total bilirubin), concomitant medications, and AEs.

Definition of an Adverse Event. AEs were tabulated by system organ class and by preferred term within system organ class according to the Medical Dictionary for Regulatory Activities (MedDRA®) Version 10. An AE was any untoward medical event that occurred to a study participant once the participant had signed the informed consent form until the study participant's last study visit, whether or not the event was considered drug-related. The severity of AEs and the characterization of Serious Adverse Events (SAEs) were evaluated using standard FDA criteria.

The relationship of AEs to the investigational drug was determined by each site investigator and was categorized as “Possibly Related” based on the following criteria: 1) the AE was reasonably related in time with LCTOPC1 exposure, the injection procedure used to administer LCTOPC1, and/or the concomitant immunosuppression administered AND 2) the AE could be explained either by exposure to the investigational product or equally well by factors or causes other than exposure to the investigational product. Adverse events were monitored by the External Medical Monitor, Sponsor Medical Monitor, and DSMB.

Neurological Assessments. The secondary endpoint was neurological function including measurement of sensory scores and lower extremity motor scores. Neurological function was evaluated using the ISNCSCI examination for motor and sensory testing and for designation of the American Spinal Injury Association (ASIA) impairment scale (AIS).

Exploratory Endpoints. Pain assessment was performed using the International Spinal Cord Injury Pain Basic Data Set. A set of three questions was added to assess allodynia. These questions covered the presence and severity of pain provoked or increased by brushing, pressure or contact with cold. Information on pain medication was collected as part of the assessment of concomitant medications. Potential exploratory endpoints for recovery of neurological function were: University of Alabama-Birmingham Index of Motor Recovery (UAB-IMR), Spinal Cord Independence Measure (SCIM), and assessment of bowel and bladder function.

Lumbar Puncture. A lumbar puncture to obtain 10 mL of cerebrospinal fluid (CSF) was conducted after receiving general anesthesia but prior to LCTOPC1 injection as well as at day 60 post-injection. The volume required at individual study sites for the following tests were sent to the hospital laboratory: white blood cell count, glucose, total protein, oligoclonal banding, myelin basic protein, and immunoglobulin G index. In addition, CSF was evaluated by the sponsor to assess immune response to LCTOPC1.

Magnetic Resonance Imaging. Screening/Baseline MRI was obtained between 3 and 5 days prior to injection (Day-3 and Day-5) of LCTOPC1 but no earlier than 4 days after SCI. Screening/baseline MRI included the brain, cerebellum, and entire spinal cord, with and without contrast (gadolinium dietheylenetriamine pentaacetic acid [Gd-DTPA]). If surgery for LCTOPC1 injection was subsequently delayed for more than 3 days, then a repeat MRI of the thoracic spine, without contrast, was obtained. Follow-up MRIs of the spinal cord and cerebellum, with and without contrast (Gd-DTPA), were obtained on Days 7, 60, 120, and 270 post-injection. A full central nervous system MRI, with and without contrast (Gd-DTPA), was obtained on Days 30, 90, 180, and 365 as well as yearly between years 2-5. Image acquisition protocols were standardized. Image review was centralized and standardized with by an independent radiologist, DD at Radiology Imaging Associates Denver.

HLA Typing and Immunological Monitoring. LCTOPC1 cells do not express Human Leukocyte Antigen (HLA) Class II and are resistant to NK cell lysis. However, one concern in regard to the safety and potential efficacy of LCTOPC1 was the possibility of allogeneic rejection by the host's immune system. Immunosuppression was minimized in terms of duration to 60 days and dosed below the typical long-term maintenance therapy levels used for solid organ transplant. Peripheral blood and cerebrospinal fluid (CSF) samples from LSTOPC1 injected participants were collected according to protocol. A lumbar puncture to obtain 10 mL of CSF was conducted after receiving general anesthesia but prior to LCTOPC1 injection as well as at day 60 post-injection to assess for rejection of allogenic cells as well as for immunologic monitoring. The following assessments occurred at the hospital laboratory: white blood cell count, glucose, total protein, oligoclonal banding, myelin basic protein, and immunoglobulin G index. Peripheral blood was examined for the presence of antibodies specific for the donor-specific HLA molecules on LCTOPC1 and to detect T cell-mediated responses to LCTOPC1. In addition, CSF was evaluated by the sponsor to further assess immune response to LCTOPC1 and for the presence of LCTOPC1 (day 60) using a PCR based assay.

Statistical Methods. Descriptive analysis was used due to the small sample size, open-label, and non-randomized study design. The primary and secondary endpoints of this study are presented descriptively in table, figure, and text form.

Results

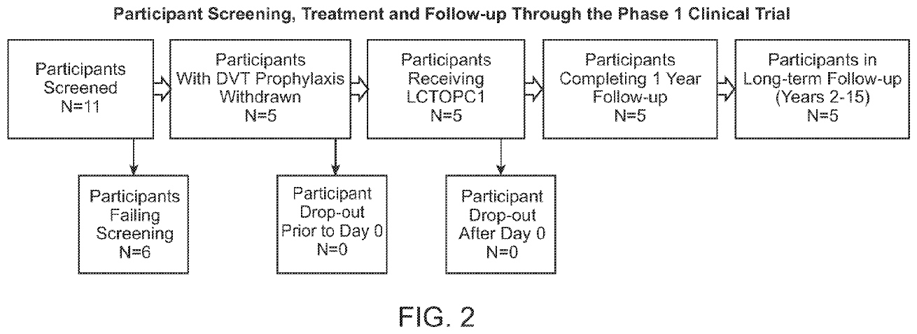

Study Participants. The first participant was implanted the winter of 2010 and the last participant was enrolled in the winter of 2011. Eleven individuals with SCI were screened for enrollment, with six individuals who failed screening: four due to MRI artifacts which prohibited adequate spinal cord visualization, one based on the ISNCSCI examination (NLI T1), and one due to elevated liver enzymes. A total of five individuals with SCI received LCTOPC1 at three study sites. FIG. 2 provides a Consolidated Standard of Reporting Trials (CONSORT) flow diagram. In this trial, the most common mechanism of injury was motor vehicle-related for four of five individuals, with a fall being the cause of injury in one individual. Four of five participants enrolled were male. The cohort age ranged from 21 to 32 years of age (Table 1).

TABLE 1
Demographic and Baseline Disease Characteristics-All Treated Subjects

| | Age | | | Weight | BMI | |
|---|---|---|---|---|---|---|
| Subject | (years) | Sex | Race | (kg) | (kg/m²) | Cause of Injury |
| 1002 | 23 | Male | Caucasian | 63.1 | 23.1 | Motor vehicle accident |
| 1003 | 23 | Mala | Caucasian | 78.4 | 23.4 | Restrained driver in rollover motor vehicle |
| | | | | | | collision with ejection |
| 1101 | 32 | Male | Caucasian | 70.5 | 22.9 | |
| 1203 | 31 | Male | Caucasian | 62.7 | 27.7 | Fall 30 feet down rock embankment |
| 1204 | 23 | Female | Caucasian | 61.5 | 24.8 | Car accident |

Abbreviations:
BMI = body mass index.
kg = kilograms, m = meter.

Participant Follow-up. As of this publication, all participants have entered their tenth year of follow-up. In agreement with the FDA, the trial was structured to begin with 5 years of in-person evaluation followed in years 6 through 15 with phone interviews. During the first 5 years of the study, 24 of 25 in-person annual visits were completed. One participant did not participate in the year 5 in-person visit but has participated in scheduled phone follow-up. From year 6 to the current time, 21 of 21 annual phone interviews have been completed. One participant has completed 10 years of follow-up and four participants are entering their 10-year follow-up interviews.

Primary Outcome Measure: Evaluation of Safety. All SAEs and AEs (related and unrelated to procedure, cell implant, or immunosuppression) are summarized in Table 2 and described below.

TABLE 2

| | Total (N = 5) | | |
|---|---|---|---|
| | Number of | | |
| System Organ Class (SOC) Preferred Term | Events | n (%) | |
| All events | 172 | | |
| Nervous system disorders | 19 | 4 | (80.0) |
| Eye disorders | 2 | 2 | (40.0) |
| Gastrointestinal disorders | 16 | 5 | (100) |
| General disorders and administration site | 8 | 3 | (60.0) |
| conditions | | | |
| Immune system disorders | 2 | 2 | (40.0) |
| Infections and infestations | 42 | 5 | (100) |
| Injury, poisoning and procedural complications | 10 | 5 | (100) |
| Investigations | 5 | 3 | (60.0) |
| Metabolism and nutrition disorders | 3 | 2 | (40.0) |
| Musculoskeletal and connective tissue disorders | 32 | 5 | (100) |
| Psychiatric disorders | 8 | 2 | (40.0) |
| Renal and urinary disorders | 7 | 4 | (80.0) |
| Reproductive system and breast disorders | 1 | 1 | (20.0) |
| Respiratory, thoracic and mediastinal disorders | 2 | 2 | (40.0) |
| Skin and subcutaneous tissue disorders | 11 | 3 | (60.0) |
| Surgical and medical procedures | 2 | 2 | (40.0) |
| Vascular disorders | 2 | 1 | (20.0) |

N = number of subjects in safety population or number of subjects with respective event category;
n = number of subjects in respective category;
% = n * 100/N.

Serious Adverse Events Related to Procedure, Cell Implant, or Immunosuppression. There were no SAEs related to the procedure, cell implant, or immunosuppression. There were no findings of clinical concern on MIRI scans of the full central nervous system through five years post-injection in any participant. During long-term phone follow-up participants denied having any fever of unknown cause, no changes in sensation in chest, arms, or legs (other than described below), and no participants have been diagnosed with any type of cancer. No participants died during the study. Safety events were monitored by the DSMB and no suspension rules were triggered.

Serious Adverse Events Unrelated to Procedure, Cell Implant, or Immunosuppression. Three participants have reported four SAEs unrelated to the procedure, cell implant, or immunosuppression. These SAEs included urinary tract infection (UTI) and subsequent transitory autonomic dysreflexia in one individual, pyelonephritis, and a mood disorder in two different individuals.

Adverse Events Categorized by Grade. Over the course of the trial, 25 AEs were judged by the Investigators to be possibly related to LCTOPC1 (Grade 1/Mild [n=9], Grade 2/Moderate [n=15], and Grade 3/Severe [n=1]). The Grade 3 AE was described as a burning sensation in the trunk and lower extremities first reported on Day 57 post-injection with Grade 1 severity, increasing to Grade 3 severity on Day 90 post-injection. This neuropathic pain resulted in three additional Grade 2 severity AEs and was ongoing through year 9 follow-up. Grade 2 AEs included: surgical site pain, hypomagnesemia, urinary tract infection, vaginal yeast infection, emesis, upper back pain, shoulder pain, pain with range of motion, and autonomic discomfort during catheterization relieved after treatment with lidocaine. Grade 1 AEs included: hypomagnesemia, urinary tract infection, fever, headache, nausea, and spasticity.

Adverse Events Categorized by Relation to Procedure, Cell Implant, or Immunosuppression. Nine of the 25 related adverse events were deemed possibly related specifically to the injection procedure. Eight of the nine were Grade 1 or 2 in severity and one was Grade 3. The AEs were predominantly transient postoperative pain, one fever, and one urinary tract infection. There were no AEs attributed to the cell implant. Moreover, the immunosuppression regiment was well tolerated, and all five participants completed the regimen per protocol. Sixteen of the 25 adverse events were deemed possibly related specifically to the immunosuppression. Seven Grade 1 AEs and nine Grade 2 AEs were judged to be possibly related specifically to tacrolimus. These AEs were primarily known common adverse reactions to tacrolimus (nausea/emesis, low magnesium level, infections). Among reported infections, 1 of 7 was a vaginal yeast infection and 6 of 7 infections were in the urinary tract, which is a common complication of SCI.

Adverse Events Unrelated to Procedure, Cell Implant, or Immunosuppression. At year 6, one participant reported an increase in frequency and intensity of muscle spasms attributed to functional electrical stimulation (FES) cycling. This participant reported resolution of these symptoms during years 7 through 9 and is currently not using any medication for muscle spasms. In year 9, a different individual received outpatient testing after developing a deep vein thrombosis (DVT).

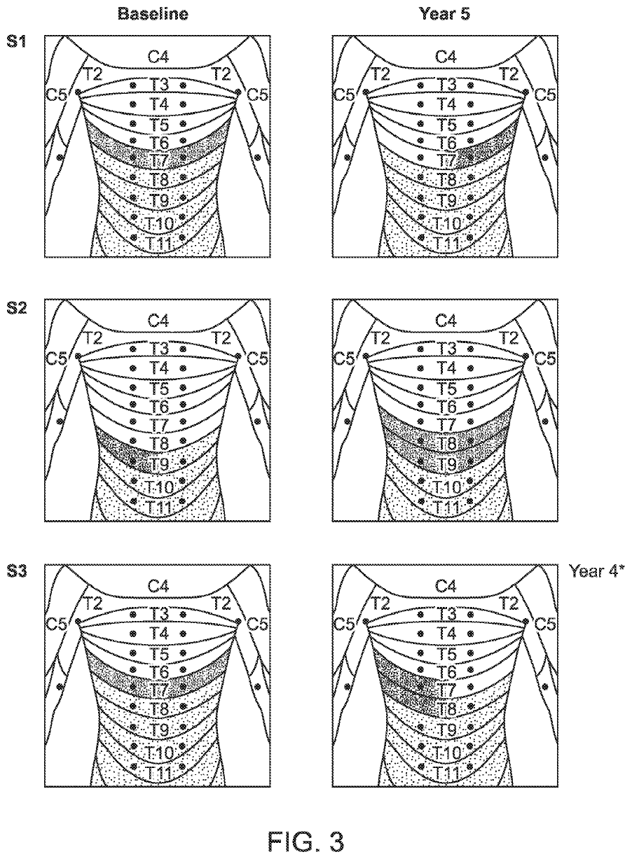

Secondary Outcome Measure: Neurological Assessment. After discharge from acute inpatient rehabilitation and through the first five years post-implantation, participants continued to be evaluated in-person according to the schedule shown in FIG. 1. Of note, between baseline and Year 5, participants' annual in-person evaluations included at least 13 ISNCSCI exams. All participants had an ASIA impairment Score (AIS) grade of A on enrollment in the trial and all participants have maintained the same impairment grade. The highest single and lowest NLI enrolled in the study were T3 and T8 respectively. Only the individual with T3 NLI improved to T4 with a sensory ZPP initially at T4 bilaterally noted to improve to T5 on the left and T6 on the right at one year follow-up. In total three of five participants experienced at least one level of improvement in their ZPP. All participants began and ended the 5 years of in-person ISNCSCI examination with intact upper extremity motor function with an upper extremity motor score (UEMS) of 50 out of 50, and lower extremity motor score (LEMS) of 0 out of 50 (Table 3). Over the course of 5 years of in-person follow-up, sensory examinations have not materially changed. FIG. 3 provides a diagrammatic representation of the motor and sensory function of each patient at baseline and at 5 years post LCTOPC1 administration.

TABLE 3
ISNCSCI Baseline, Year 1 post-transplantation, and Year 5 post-transplantation

| | | | | | | | | | | | Zone of Partial | | | | |
|---|---|---|---|---|---|---|---|---|---|---|---|---|---|---|---|
| | | | | | | Neurologic Level | | | | | Preservation | | | | |
| | Visit | | | | | Sensory | | Motor | | Single | Sensory | | Motor | | |
| Subjec | Study | Date | TSS | UEM | LEMS | Right | Left | Right | Left | Score | Right | Left | Right | Lett | AIS |
| 1002 | Baseline | 6 Oct. 2010 | ND | ND | 0 | T6 | ND | T6 | ND | ND | T7 | T7 | T6 | ND | A |
| | Year 1 | 6 Oct. 2011 | 111 | 50 | 0 | T6 | T7 | T6 | T7 | T6 | T7 | T7 | T6 | T7 | A |
| | Year 5 | 6 Oct. 2015 | 111 | 50 | 0 | T7 | T6 | T7 | T6 | T6 | T7 | T7 | T7 | T6 | A |
| 1003 | Baseline | 7 Aug. 2011 | 125 | 50 | 0 | T8 | T8 | T8 | T8 | T8 | T9 | T9 | T8 | T8 | A |
| | Day 270* | 3 May 2012 | 129 | 50 | 0 | T8 | T8 | T8 | T8 | T8 | T10 | T10 | T8 | T8 | A |
| | Year 5 | 2 Aug. 2016 | 122 | 50 | 0 | T7 | T7 | T7 | T7 | T7 | T10 | T10 | T7 | T7 | A |
| 1101 | Baseline | 5 May 2011 | 112 | 50 | 0 | T6 | T6 | T6 | T6 | T6 | T8 | T8 | T6 | T6 | A |
| | Year 1 | 24 Apr. 2012 | 112 | 50 | 0 | T6 | T6 | T6 | T6 | T6 | T8 | T8 | T6 | T6 | A |
| | Year 4* | 4 May 2015 | 114 | 50 | 0 | T6 | T7 | T6 | T7 | T6 | T8 | T8 | T6 | T7 | A |
| 1203 | Baseline | 15 Sep. 2011 | 121 | 50 | 0 | T7 | T8 | T7 | T8 | T7 | T8 | T9 | T7 | T8 | A |
| | Year 1 | 18 Sep. 2012 | 123 | 50 | 0 | T7 | T8 | T7 | T8 | T7 | T9 | T10 | T7 | T8 | A |
| | Year 5 | 17 Oct. 2016 | 123 | 50 | 0 | T7 | T8 | T7 | T8 | T7 | T9 | T9 | T7 | T8 | A |
| 1204 | Baseline | 14 Nov. 2011 | 82 | 50 | 0 | T3 | T3 | T3 | T3 | T3 | T4 | T4 | T3 | T3 | A |
| | Year 1 | 13 Nov. 2012 | 95 | 50 | 0 | T4 | T4 | T4 | T4 | T4 | T6 | T5 | T4 | T4 | A |
| | Year 5 | 31 Oct. 2016 | 97 | 50 | 0 | T4 | T5 | T4 | T5 | T4 | T6 | T6 | T4 | T6 | A |

TSS = Total Sensory Score;
*participants were not able to follow u
Table 3 presents total sensory score (TSS), upper extremity motor score (UEMS), lower extremity motor score (LEMS), sensory neurological level of injury (NLI), motor NLI, sensory zone of partial preservation (ZPP), Motor ZPP, and ASIA impairment Score (AIS) grade at baseline, year 1 and year 5. All five individuals were AIS Grade A at enrollment and there were no conversions to AIS B. The highest single and lowest NIL enrolled in the study were T3 and T8 respectively. Only the individual with the T3 NLI improved to T4 with a sensory ZPP initially at T4 bilaterally noted to improve to T5 on the left and T6 on the right at one year follow up. In total three of five participants experienced at least 1 level of improvement in their ZPP. ND = Unable to determine.

MRI Findings. No participant exhibited evidence of an enlarging cyst, enlarging mass, spinal cord damage related to the injection procedure, intramedullary hemorrhage, CSF leak, epidural abscess or infection, inflammatory lesions in the spinal cord, CSF flow obstruction, or masses in the ventricular system. No evidence of any adverse neurological changes or adverse changes on MRI was reported during tacrolimus tapering or after tacrolimus discontinuation.

Immune Monitoring. LSTOPC1 is an allogeneic cell therapy and is potentially sensitive to rejection by the recipient immune system. As a baseline assessment, HLA Class I and Class II molecular typing was performed for 10 alleles of the donor LCTOPC1 cells and peripheral blood cells of each of the 5 recipients. The potential development of a cellular immune response to LCTOPC1 was assessed and showed no evidence of T-cell mediated responses to LCTOPC1 even after cessation of tacrolimus immunosuppression in any of the serum samples of the five recipients. In addition, CSF samples obtained through lumbar puncture did not show signs of antibody or T-cell responses to LCTOPC1.

### DISCUSSION

In January of 2009, Nature reported that LCTOPC1 would enter “the world's first clinical trial of a therapy generated by human embryonic stem cells”. At the time, pharmaceutical research in acute SCI was considered a relatively recent development. Although no clinical trial of hESC-derived cell lines had ever been assessed in any context, procedures for other intraparenchymal injections of cellular products (e.g. activated autologous macrophages) into the spinal cord had been evaluated providing a partial roadmap for LCTOPC1 based studies.

We present the primary and secondary outcome measures of five participants who received 2×10⁶ allogeneic hESC derived oligodendrocyte progenitor cells within 7-14 days post-injury. The primary results from the first 10 years of follow-up establish the safety and feasibility of intraparenchymal LCTOPC1 injection. The injection procedure and the low-dose immunosuppression regimen were well tolerated. To date, all five participants who received LCTOPC1 demonstrate no evidence of neurological deterioration or adverse findings on MRI scans.

This study was not designed to assess efficacy; however, animal studies of LCTOPC1 produced improvements in motor function through mechanisms that appeared to represent remyelination as well as neuroprotection, suppression of inflammation, promotion of axonal regeneration, and/or homeostatic maintenance. The proposed mechanism of locomotor function improvement included remyelination as well as expression of neurotrophic factors. The limited signs of functional recovery in various human trials despite promising results in animals may be related to the relative severity of human injuries in comparison to preclinical studies with incomplete contusion, suggesting that subsequent studies with incomplete injuries may demonstrate recovery more similar to that seen in animal models. Although this study did not demonstrate significant recovery, no participant exhibited evidence of neurological deterioration on ISNCSCI examinations through 5 years of in-person follow-up or 10 years of self-reported neurological function. The total motor score as well as the total sensory score of the ISNCSCI exam have remained stable across time. No unanticipated SAEs related to LCTOPC1 have been reported with 98% follow-up of participants (45 of 46 annual visits) through the first 10 years of the clinical trial.

Neuropathic pain in response to LCTOPC1 secondary to remyelination or neurotrophic factors was assessed using the International Spinal Cord Injury Pain Basic Data Set and a set of three questions to assess allodynia. Neuropathic at level pain and below level pain often have onset during the first several months after SCI, and by 1 year the prevalence of neuropathic pain approaches 60%. The prevalence of pain in this study is consistent with the natural history of neuropathic pain. One participant experienced neuropathic pain reported as a burning sensation in the trunk and lower extremities that was considered possibly related to LCTOPC1, which persisted in long-term follow up. The pain reported by this participant is consistent with two of the major categories of pain that are common following SCI: neuropathic pain at the level of injury (termed neuropathic at level pain), and neuropathic pain that occurs diffusely below the level of injury (termed neuropathic below level pain). Unfortunately for affected individuals, both at level and below level neuropathic pain are often severe and persistent for at least 5 years after SCI, despite attempts at pain management. In addition, 40 to 50% of individuals with these types of pain report their pain as severe or excruciating. It is not possible to determine a cause-and-effect relationship between LCTOPC1 or change in the incidence of long-term neuropathic pain in this small open-label study.

Serial MRI studies did not demonstrate the formation of ectopic tissue and/or teratomas. In addition to the absence of space occupying lesions, the natural history of chronic SCI MRI studies suggests that cavitary lesions will be identifiable in 58% of individuals who pursue thoracic level cellular trials. MRI results during the long-term follow-up period for LCTOPC1 were of particular significance because 80% of individuals showed T2 signal changes consistent with the formation of a tissue matrix at the injury site. Although the sample size is limited, these findings suggest that LCTOPC1 cells may have either durable engraftment and/or induced long-term changes which limited cavitation at the injury site.

SCI is a relatively rare condition and the potential population of T3-T11 AIS A injuries represent less than 20% of acute SCI in the United States (NSCISC National Spinal Cord Injury Statistical Center, University of Alabama at Birmingham, 2011 Annual Statistical Report—Complete Public Version).

### CONCLUSION

The LCTOPC1 thoracic SCI clinical trial is one of the longest running clinical trials in the hESC field. The study provides crucial first-in-human positive safety data for future hESC derived therapies. While we cannot exclude the possibility of future adverse events, the experience in this trial provides evidence that these treatments can be well tolerated and event-free for up to 10 years. In addition, this report supports the willingness of participants to participate in long-term follow-up as well as setting a standard for corporate sponsors' commitment to data collection beyond their immediate financial interests. Based on the safety profile of LCTOPC1 obtained in this study, a cervical dose escalation trial was initiated.

Stem Cell Materials and Methods

The following Examples provide exemplary methods for obtaining cells that can be used in the methods and uses described herein. Additional methods, materials, and uses can be found, for example, in WO/2020/154533, which is incorporated herein by reference in its entirety.

Example 2—Culture and expansion of undifferentiated human embryonic stem cells.

Undifferentiated human embryonic stem cells (uhESC) from a working cell bank (WCB) generated from the H1 line (WA01; Thomson J A, Itskovitz-Eldor J, Shapiro S S, Waknitz M A, Swiergiel J J, Marshall V S, Jones J M. Embryonic stem cell lines derived from human blastocysts. Science. 1998 Nov. 6; 282(5391):1145-7) were cultured on recombinant human Laminin-521 (rhLn-521, Corning #354224) coated, tissue culture treated polystyrene T-75 culture flasks (Corning #431082) in complete mTeSR™-1 medium (Stem Cell Technologies #85850). The medium was completely exchanged daily until the cells reached approximately 80-90% confluency, and uhESCs were then passaged using ReLeSR™ reagent (Stem Cell Technologies #05872). ReLeSR™-lifted uhESC cells were seeded in new rhLn-521 coated 225 cm² flasks, and daily medium exchange was resumed two days post-seeding. Cultured uhESCs from the WCB were expanded in this manner for between two to five passages, depending on the experiment, prior to differentiation into neuroectoderm progenitor cells as described in Example 3.

Example 3—Method of differentiating human embryonic stem cells to neuroectoderm progenitors with dorsal spinal cord progenitor phenotype.

Expanded uhESC were seeded on rhLn-521-coated vessels, and cultured until reaching 40-70% confluency at which point differentiation was initiated. Days 0-3: Differentiation was initiated by complete removal of mTeSR™-1 medium, and addition of Glial Progenitor Medium (GPM; consisting of DMEM/F12 (Gibco Catalog No. 10565-018) supplemented with 2% B27 supplement (Gibco Catalog No. 17504-044), and 0.04 μg/mL tri-iodo-thyronine (Sigma-Aldrich Catalog No. T5516-1MG)) supplemented with 10 μM of MAPK/ERK inhibitor, PD0325901 (PD; Sigma-Aldrich Catalog No. PZ0162), 2 μM of BMP signaling inhibitor, Dorsomorphin (Dorso; Sigma-Aldrich Catalog No. P5499), and 1 μM of Retinoic Acid (RA; Sigma-Aldrich Catalog No. R2625). This medium was replenished daily. Days 4-6: On Day 4, culture medium was switched to GPM supplemented with 1 μM RA and 150 μM Ascorbic Acid (Sigma-Aldrich Catalog No. A4544) and replenished daily. Day 7: On Day 7, the cells were harvested for expansion and further differentiation into glial progenitors as described in Example 4. A subset of cells were collected for analysis by quantitative PCR (qPCR; as described in Example 7), flow cytometry (as described in Example 6), and when available, separate well plates set aside for analysis were prepared for immunocytochemistry (ICC) (as described in Example 6). At Day 7, these cells exhibited marker expression consistent with dorsal spinal cord progenitors (Table 4).

TABLE 4
qPCR analysis of gene markers for pluripotency and neuroectoderm progenitor cells (NPCs) in
H1 uhESCs differentiated into NPCs using different combinations of small molecule inhibitors.

| | | | | | |
|---|---|---|---|---|---|
| PD0325901 + | AZD6244 + | GSK1120212 + | PD184352 + | Cobimetinib + | PD0325901 + |
| Dorso | Dorso | Dorso | Dorso | Dorso | ML347 |

Pluripotency genes

| | | | | | | |
|---|---|---|---|---|---|---|
| NANOG | 1 | 4 | 1 | 1 | 1 | 1 |
| LIN28A | 548 | 618 | 606 | 635 | 504 | 488 |
| SOX2 | 370 | 484 | 254 | 401 | 201 | 202 |

Neuroectoderm progenitor cell genes

| | | | | | | |
|---|---|---|---|---|---|---|
| PAX6 | 363 | 539 | 258 | 417 | 173 | 156 |
| HES5 | 97 | 100 | 68 | 91 | 36 | 36 |
| ZBTB16 | 135 | 149 | 145 | 171 | 134 | 120 |

Dorsal spinal cord progenitor cell genes

| | | | | | | |
|---|---|---|---|---|---|---|
| TFAP2A | 67 | 61 | 84 | 43 | 100 | 166 |
| PAX3 | 112 | 99 | 180 | 84 | 193 | 158 |
| PAX7 | 156 | 106 | 186 | 100 | 148 | 209 |

Ventral spinal cord progenitor cell genes

| | | | | | | |
|---|---|---|---|---|---|---|
| OLIG2 | 1 | 1 | 1 | 1 | 1 | 1 |
| NKX2-2 | 1 | 1 | 1 | 1 | 1 | 1 |

Example 4—Method of differentiating human embryonic stem cells to glial lineage cells.

Days 7-13: Differentiation of uhESCs to neuroectoderm progenitors, specifically of a dorsal phenotype, was performed as described in Example 3. On Day 7, the cells were lifted using TrypLE™ Select (Thermo Fisher, cat #A12859-01), counted, and seeded onto rhLn-521-coated vessels at a seeding density of 2.7×10⁴ cells/cm² in GPM supplemented with 20 ng/mL human basic fibroblast growth factor (hbFGF, Thermo Fisher, cat #PHG0263), 10 ng/mL epidermal growth factor (EGF, Thermo Fisher, cat #PHG0311), and 10 μM Rho Kinase Inhibitor (RI, Tocris Catalog No. 1254). Culture medium was replenished daily by aspirating spent medium and replacing it with fresh GPM+hbFGF+EGF. Days 14-21: At Day 14, cells were lifted using TrypLE™ Select, counted, resuspended in GPM+hbFGF+EGF+RI, and reseeded in dynamic suspension cultures at a density of 1.83×10⁶ viable cells/mL into either PBS-0.1 L or PBS-0.5 L Mini Bioreactor Systems (PBS Biotech). Subsets of cells at Day 14 were collected for analysis by flow cytometry (Example 6), ICC (Example 6), and qPCR (Example 7). PBS0.1 L and PBS0.5 L Mini Bioreactors were set to rotate at 35 RPM and 28 RPM, respectively. Culture medium was replenished daily by allowing the aggregates to settle, removing 70-80% of spent medium, and replacing with an equal volume of GPM+bhFGF+EGF. On Day 15, the rotation velocity was increased to 45 RPM and 32 RPM for the PBS0.1 L and PBS0.5 L Mini Bioreactors, respectively. At Day 21, subsets of the aggregates were collected for ICC (Example 6) and qPCR (Example 7). By Day 21, the differentiated cells expressed markers consistent with glial-restricted cells (Table 4).

Example 5—Method of differentiating human embryonic stem cells to oligodendrocyte progenitor cells.

Days 21-42: The glial-restricted progenitor cells obtained in Example 4 were further differentiated into oligodendrocyte progenitor cells (OPCs). The differentiation protocol for Days 0-20 was performed as described in Examples 3 and 4. On Day 21, aggregates were transferred from dynamic suspension to rhLn-521-coated culture vessels. For example, starting with 1× PBS-0.1 L Mini Bioreactor with 60 mL of total volume, the 60 mL of culture was split onto 2×T75 flasks, each with 30 mL of volume. Subsequently, cells were fed every other day with GPM supplemented with 20 ng/mL EGF and 10 ng/mL of platelet-derived growth factor AA (PDGFAA; PeproTech, cat #AF-100-13A). Every seven days, (i.e., Day 28 and Day 35), cells were lifted with TrypLE™ Select, counted, and reseeded onto fresh rhLn-521-coated culture vessels at a seeding density of 4×10⁴ viable cells/cm². The differentiated cells were harvested on Day 42. Cells were detached from vessels using TrypLE™ Select, counted, and re-formulated in CryoStor10 (BioLife Solutions, cat #210102) prior to cryopreservation. Subsets of cells were collected for analysis by flow cytometry (Example 6), ICC (Example 6), and qPCR (Example 7). By Day 42, the differentiated cells expressed markers characteristic with OPCs as measured by the three analytical methods (Table 4).

Example 6—Characterization of differentiated cell populations by immunocytochemistry and flow cytometry.

Flow cytometry and immunocytochemistry (ICC) can be used to detect and characterize different aspects of protein marker expression in a cell population. While flow cytometry can be used to quantify the percentage of individual cells within the population that exhibit a given protein marker profile, ICC provides additional information about the subcellular localization of each protein marker and can be applied to single cells or cellular aggregates. By using either or both of these protein profiling approaches, we tracked the differentiation of human embryonic stem cells to neuroectoderm progenitor cells, glial progenitor cells, and oligodendrocyte progenitor cells according to the methods of the present disclosure. For human embryonic stem cells differentiated into neuroectoderm progenitor cells and glial progenitor cells, protein marker expression in the differentiated Day 7 and Day 21 cells was characterized by ICC. Adherent cells and cellular aggregates were fixed in 4% paraformaldehyde (PFA) for 30 minutes at room temperature (RT). Fixed cells and aggregates were washed with phosphate buffered saline (PBS), and fixed aggregates were then sequentially placed in increasing concentrations of sucrose solution (10%, 20%, and 30% weight/volume) for 30 minutes at RT, 30 minutes at RT, and overnight at 4° C., respectively. Following sucrose replacement, aggregates were embedded in Tissue-Tek Optimal Cutting Temperature (OCT) solution (Sakura Finetek USA #4583) and frozen at −80° C. OCT-embedded aggregates were warmed to −20° C., cut into 30 μm sections using a cryostat (model CM3050 S, Leica Biosystems, Buffalo Grove, IL, USA), and mounted onto poly-L-lysine (Sigma-Aldrich #P4707) coated glass slides.

To perform immunocytochemical staining, fixed adherent cells and slide-mounted aggregate sections were permeabilized and blocked in blocking solution consisting of 0.1% Triton™ X-100/2% normal goat serum/1% bovine serum albumin in PBS for 2 hours at room temperature (RT). Following permeabilization and blocking, adherent cells and aggregate sections were incubated overnight at 4° C. in blocking solution without Triton™ X-100 and containing primary antibodies specific to protein markers of interest, including PAX6 (BD Pharmingen #561462 or BioLegend #901301) to detect neuroectoderm progenitors, and AP2 (Developmental Studies Hybridoma Bank—DSHB #3B5), PAX3 (DSHB #Pax3), and PAX7 (DSHB #Pax7) to detect dorsal spinal cord progenitor cells. Adherent cells and aggregate sections were then washed three times with PBS followed by incubation with secondary antibodies specific to the chosen primary antibodies and 4′,6-diamidino-2-phenylindole (DAPI) counter-stain in blocking solution without Triton™ X-100 for 1 hour at RT protected from light. Adherent cells and aggregate sections were washed three times with PBS and imaged using an IN Cell Analyzer 2000 (GE Healthcare, Pittsburgh, PA, USA).

After 7 days of differentiation, the adherent cell population from two representative experiments expressed PAX6, a protein marker characteristic of neuroectoderm progenitor cells and also expressed the dorsal spinal cord progenitor markers, AP2, PAX3, and PAX7.

Aggregates were sectioned and stained for dorsal progenitor markers AP2, PAX3, and PAX7, as well as pan-neural progenitor marker PAX6. While these early progenitor cells were still present at Day 21, also present was a distinct glial population expressing the oligodendrocyte progenitor marker NG2.

For human embryonic stem cells differentiated through Day 42 into oligodendrocyte progenitor cells, protein marker expression in the resulting single cell population was characterized by both flow cytometry and ICC. To characterize protein marker expression of the oligodendrocyte progenitor cells by ICC, staining was carried out as described above for slide-mounted aggregate sections, except permeabilization was performed with 100% methanol for 2 minutes at RT, and blocking solution consisted of 10% fetal bovine serum in PBS.

Based on ICC data for the Day 42 oligodendrocyte progenitor cells, the resulting single cell population from two representative experiments expressed the oligodendrocyte progenitor cell marker NG2 and reduced expression of the dorsal spinal cord progenitor cell marker AP2.

To quantify cell surface markers on Day 42 by flow cytometry, cells were thawed in Thaw Medium (10% fetal bovine serum in DMEM medium), centrifuged and resuspended in Stain Buffer (2% fetal bovine serum/0.05% sodium azide in PBS). Cells were incubated with primary antibodies specific to markers of interest, including NG2 (Invitrogen #37-2300), PDGFRα (BD Biosciences #563575), GD3 (Millipore #MAB2053), A2B5 (BD #563775), CD49f (Millipore #CBL458P), EpCAM (Dako #M080401-2) and CLDN6 (Thermo Fisher #MA5-24076), and their isotype controls for 30 minutes on ice. Cells were washed with Stain Buffer to remove unbound antibodies; in the case of unconjugated antibodies, cells were then incubated with appropriate fluorophore-conjugated secondary antibodies for 30 minutes on ice. Cells were washed and propidium iodide was then added to demark dead cells. In some cases, cells were cultured overnight at 37° C./5% CO₂ in tissue culture vessels coated with Matrigel (Corning #356231) to recover protein markers that exhibited sensitivity to the Day 42 harvesting procedure described in Example 5, and were then harvested with TrypLE™ Select (Thermo Fisher #A12859-01) and stained for flow cytometry analysis as described above. All cells were analyzed on an Attune NxT (Thermo Fisher, Waltham, MA, USA) flow cytometer. To calculate the percentage of cells expressing a given protein marker, dead cells staining with propidium iodine were gated and the number of viable cells bound to the corresponding antibody was expressed as a fraction of the total number of cells analyzed after correcting for the number of cells that exhibited non-specific binding to the isotype control antibody.

Table 5 shows representative flow cytometry data for Day 42 oligodendrocyte progenitor cells generated in accordance with the methodology described in Example 5. As shown for two representative runs, a high proportion of cells in the resulting cell population expressed characteristic oligodendrocyte markers, including NG2 and PDGFRα. In addition, non-OPC markers were minimally detected in the resulting population, including the neural progenitor/epithelial marker CD49f and the epithelial markers CLDN6 and EpCAM.

TABLE 5
Representative flow cytometry data for oligodendrocyte
progenitor cells produced by a method in accordance
with the present disclosure.

| | OPC/Pre-OPC markers | | | | Non-OPC markers | | |
|---|---|---|---|---|---|---|---|
| | NG2 | PDGFRα | GD3 | A2B5 | CD49f | CLDN6 | EpCAM |
| Run 1 | 92% | 85% | 66% | 39% | 3% | 0% | 0% |
| Run 2 | 98% | 95% | 63% | 89% | 0% | 0% | 2% |

The cell population generated by the methodology described in the present disclosure resulted in higher proportion of cells positive for oligodendrocyte progenitor cell marker NG2 and reduced expression of non-OPC markers CD49f, CLDN6, and EpCAM when compared to OPCs that are currently in clinical testing to treat spinal cord injury and that were generated using another method (Priest C A, Manley N C, Denham J, Wirth ED 3rd, Lebkowski J S. Preclinical safety of human embryonic stem cell-derived oligodendrocyte progenitors supporting clinical trials in spinal cord injury. Regen Med. 2015 Nov.; 10(8):939-58; Manley N C, Priest C A, Denham J, Wirth ED 3rd, Lebkowski J S. Human Embryonic Stem Cell-Derived Oligodendrocyte Progenitor Cells: Preclinical Efficacy and Safety in Cervical Spinal Cord Injury. Stem Cells Transl Med. 2017 October; 6(10):1917-1929).

Example 7—Characterization of differentiated cell populations by gene expression profiling.

Gene expression profiling can be used to characterize the cellular phenotype of the starting pluripotent cell population and each stage of differentiation, including the generation of neuroectoderm progenitor cells, glial progenitor cells, and oligodendrocyte progenitor cells. Gene expression profiling includes both global transcriptome profiling, using such methods as microarray and RNA-seq, and targeted gene profiling using methods of increased sensitivity such as quantitative real-time PCR (qPCR). To perform gene expression profiling, cells were lysed in Qiagen RLT Lysis Buffer (Qiagen #79216), and RNA was purified using Qiagen RNeasy Mini Kit (Qiagen #74106) according to the manufacturer's guidelines. For qPCR-based analysis, purified RNA was then converted to cDNA according to standard methods using the Invitrogen Superscript IV VILO Mastermix (Thermo Fisher Scientific #11756050) according to the manufacturer's guidelines. The relative expression level of target genes and reference housekeeping genes was then quantified using gene-specific primer-probe sets (Applied Biosystems Taqman Gene Expression Assays, Thermo Fisher Scientific #4331182) according to the manufacturer's guidelines. To determine relative expression levels of a given set of target genes, PCR reactions were performed on the ABI 7900HT Real-Time Sequence Detection System (Applied Biosystems), the BioMark HD System (Fluidigm) or equivalent. Each target gene was normalized to one or multiple reference genes, such as GAPDH, to determine its relative expression level.

Table 6 shows qPCR results from two representative experiments measuring expression of pluripotency genes, neuroectoderm progenitor cell genes, glial progenitor cell genes, dorsal spinal cord progenitor cell genes, ventral spinal cord progenitor cell genes, and oligodendrocyte progenitor cell genes in cell populations generated by methods in accordance with the present disclosure. RNA samples were collected at the following time points: prior to differentiation (Day 0), following differentiation to neuroectoderm progenitors (Day 7), following differentiation to glial progenitors (Day 21), and following differentiation to oligodendrocyte progenitors (Day 42). RNA samples were processed for qPCR using the methods described above. A selected panel of genes indicative of each differentiation state were quantified, including: three pluripotency genes (NANOG, LIN28A, SOX2), three neuroectoderm progenitor genes (PAX6, HES5, ZBTB16), three glial progenitor genes (CACGN4, DCC, FABP7), and three oligodendrocyte progenitor genes (CSPG4, PDGFRα, DCN). For each gene, normalized ΔCT values were calculated using the average of five housekeeping genes (ACTB, GAPDH, EP300, PGK1, SMAD1), and fold expression relative to baseline (expression below the limit of quantification) was calculated using the ΔΔCT method.

TABLE 6
qPCR analysis of gene markers for pluripotency, neuroectoderm progenitor cells
(NPCs), dorsal spinal cord progenitor cells, ventral spinal cord progenitor cells,
glial progenitor cells (GPCs), and oligodendrocyte progenitor cells (OPCs) in
H1 uhESCs differentiated into OPCs in accordance with the present disclosure.

| | | | | | | | |
|---|---|---|---|---|---|---|---|
| Run 1 | Run 2 | Run 1 | Run 2 | Run 1 | Run 2 | Run 1 | Run 2 |
| Day 0 | Day 0 | Day 7 | Day 7 | Day 21 | Day 21 | Day 42 | Day 42 |
| uhESC | uhESC | NEPC | NEPC | GPC | GPC | OPC | OPC |

Pluripotency genes

| | | | | | | | | |
|---|---|---|---|---|---|---|---|---|
| NANOG | 158 | 119 | 4 | 3 | 2 | 1 | 2 | 1 |
| LIN28A | 650 | 1684 | 233 | 626 | 5 | 1 | 2 | 1 |
| SOX2 | 480 | 325 | 532 | 243 | 767 | 262 | 2 | 3 |

Neuroectoderm progenitor cell (NPC) genes

| | | | | | | | | |
|---|---|---|---|---|---|---|---|---|
| PAX6 | 1 | 1 | 305 | 250 | 218 | 32 | 2 | 2 |
| HES5 | 1 | 1 | 65 | 71 | 275 | 298 | 6 | 1 |
| ZBTB16 | 1 | 1 | 110 | 120 | 50 | 31 | 2 | 1 |

Dorsal spinal cord progenitor cell genes

| | | | | | | | | |
|---|---|---|---|---|---|---|---|---|
| TFAP2A | 1 | 1 | 67 | 84 | 169 | 215 | 69 | 20 |
| PAX3 | 1 | 1 | 100 | 300 | 91 | 118 | 2 | 7 |
| PAX7 | 1 | 1 | 74 | 178 | 104 | 89 | 19 | 6 |

Ventral spinal cord progenitor cell genes

| | | | | | | | | |
|---|---|---|---|---|---|---|---|---|
| OLIG2 | 1 | 1 | 1 | 1 | 2 | 1 | 2 | 1 |
| NKX2-2 | 1 | 1 | 1 | 1 | 2 | 1 | 2 | 1 |

Glial progenitor cell (GPC) genes

| | | | | | | | | |
|---|---|---|---|---|---|---|---|---|
| CACNG4 | 18 | 11 | 61 | 48 | 268 | 317 | 32 | 15 |
| DCC | 1 | 2 | 17 | 51 | 34 | 324 | 2 | 5 |
| FABP7 | 6 | 10 | 7 | 9 | 17 | 51 | 2 | 4 |

Oligodendrocyte progenitor cell (OPC) genes

| | | | | | | | | |
|---|---|---|---|---|---|---|---|---|
| CSPG4 | 5 | 3 | 5 | 10 | 6 | 49 | 126 | 111 |
| PDGFRα | 1 | 2 | 19 | 20 | 23 | 185 | 584 | 481 |
| DCN | 1 | 1 | 1 | 2 | 285 | 302 | 1080 | 1136 |

Referring to Table 6, differentiation of uhESCs for seven days by a method in accordance with the present disclosure resulted in a gene expression profile that was consistent with neuroectoderm progenitor cells, including downregulation of NANOG, and expression of LIN28A, SOX2, PAX6, HES5, and ZBTB16.

In addition, the neuroectoderm progenitor cells generated after seven days of differentiation exhibited a phenotype that was consistent with dorsal spinal cord progenitor cells based on expression of the dorsal markers TFAP2A (also known as AP2), PAX3, and PAX7. As further evidence of a dorsal spinal cord progenitor cell phenotype, the resulting neuroectoderm progenitor cells did not express the ventral spinal cord progenitor cell markers OLIG2 or NKX2-2, whose expression require activation of the sonic hedgehog signaling pathway.

After 21 days of differentiation, the resulting cell population exhibited a gene expression profile that was consistent with glial progenitor cells, including downregulation of pluripotency and neuroectoderm progenitor cell markers and induction of CACNG4, DCC (also known as the netrin receptor), and FABP7. As further evidence of a glial progenitor phenotype, the resulting Day 21 cells exhibited sustained expression of HES5, which in addition to its expression in neuroectoderm progenitor cells/neural progenitor cells, HES5 has also been shown to promote the neural to glial progenitor switch in the mammalian developing central nervous system. In addition, the resulting Day 21 glial progenitor cells exhibited sustained expression of the dorsal spinal cord progenitor markers, TFAP2A, PAX3 and PAX7, providing further evidence of derivation from dorsally-patterned neural progenitors.

Following 42 days of differentiation in accordance with the methods described in the present disclosure, the resulting cell population expressed markers consistent with oligodendrocyte progenitors, including downregulation of both the earlier lineage markers and dorsal spinal cord progenitor markers, and induction of CSPG4 (also known as NG2), PDGFRα, and DCN.

Example 8—Differentiation of human embryonic stem cells to dorsal neuroectoderm progenitor cells using alternative small molecule inhibitors of MAPK/ERK and BMP signaling.

In addition to the small molecule inhibitors used in Example 3 (PD0325901 and Dorsomorphin), alternative small molecule inhibitors of MAPK/ERK and BMP signaling were tested for their ability to differentiate human embryonic stem cells into dorsal neuroectoderm progenitors. Table 7 lists the alternative small molecule inhibitors that were tested. Each condition was tested in duplicate wells of a 6-well tissue culture plate.

TABLE 7
Small molecule inhibitors used to differentiate human embryonic
stem cells into dorsal neuroectoderm progenitors.

| | | | |
|---|---|---|---|
| | Concentration | Primary binding | |
| Inhibitor | tested | target(s) | Vendor catalogue # |

MEK-ERK inhibitors

| | | | |
|---|---|---|---|
| PD0325901 | 10 μM | MEK1, MEK2 | ApexBio # A3013 |
| AZD6244 | 10 μM | MEK1, MEK2 | ApexBio # A8207 |
| GSK1120212 | 10 μM | MEK1, MEK2 | ApexBio # A3018 |
| PD184352 | 10 μM | MEK1, MEK2 | ApexBio # A1792 |
| Cobimetinib | 10 μM | MEK1, MEK2 | ApexBio # A3321 |

BMP inhibitors

| | | | |
|---|---|---|---|
| Dorsomorphin | 2 μM | ALK2, ALK3, | Sigma-Aldrich # |
| | | ALK6 | P5499 |
| ML347 | 2 μM | ALK2, ALK1 | ApexBio # B3688 |

On differentiation Day 7, cells were collected and processed for RNA extraction and gene expression profiling by qPCR as described in Example 7. For each gene, a normalized ΔCT value was calculated relative to the average of five housekeeping genes (ACTB, GAPDH, EP300, PGK1, SMAD1), and fold expression relative to baseline (expression below the limit of quantification) was calculated using the ΔΔCT method. Table 6 shows the average of fold expression value for biological duplicates of each small molecule combination (relative to baseline). Referring to Table 7, differentiation of uhESCs for seven days with each of the tested small molecule combinations resulted in downregulation of the pluripotency marker NANOG and a similar degree of maintained expression or induction of genes associated with a neuroectoderm progenitor cell phenotype, including LIN28A, SOX2, PAX6, HES5, and ZBTB16. In addition, each of the tested small molecule combinations resulted in a dorsal spinal cord progenitor phenotype based on expression of the dorsal markers, TFAP2A, PAX3, and PAX7, and a lack of expression of the ventral markers, OLIG2 and NKX2-2.

To obtain a more comprehensive comparison of the resulting Day 7 cellular phenotypes after treatment with each small molecule combination, Fluidigm qPCR was conducted using a 96 gene panel that consisted of known markers for pluripotency, neuroectoderm progenitor cells, neural tube patterning, glial progenitor cells, oligodendrocyte progenitor cells, neural crest cells, neurons, astrocytes, pericytes, Schwann cells, and epithelial cells. Comparison of the day 7 cellular phenotype for each alternative small molecule combination to the cellular phenotype generated by treatment with PD0325901 plus Dorsomorphin by regression plot of the normalized ΔCT values indicated that a similar overall cellular phenotype could be achieved with each of the small molecule combinations tested. Taken together, the results shown in Table 8 support that various combinations of: (i) a MAPK/ERK inhibitor, together with (ii) a BMP signaling inhibitor, (iii) in the absence of a SHH signaling activator, can be used to differentiate uhESCs to dorsal neuroectoderm progenitor cells, and further to glial progenitor cells and to oligodendrocyte progenitor cells using the methods of the present disclosure.

TABLE 8
qPCR analysis of gene markers for pluripotency and neuroectoderm progenitor cells (NPCs) in
H1 uhESCs differentiated into NPCs using different combinations of small molecule inhibitors.

| | | | | | |
|---|---|---|---|---|---|
| PD0325901 + | AZD6244 + | GSK1120212 + | PD184352 + | Cobimetinib + | PD0325901 + |
| Dorso | Dorso | Dorso | Dorso | Dorso | ML347 |

Pluripotency genes

| | | | | | | |
|---|---|---|---|---|---|---|
| NANOG | 1 | 4 | 1 | 1 | 1 | 1 |
| LIN28A | 548 | 618 | 606 | 635 | 504 | 488 |
| SOX2 | 370 | 484 | 254 | 401 | 201 | 202 |

Neuroectoderm progenitor cell genes

| | | | | | | |
|---|---|---|---|---|---|---|
| PAX6 | 363 | 539 | 258 | 417 | 173 | 156 |
| HES5 | 97 | 100 | 68 | 91 | 36 | 36 |
| ZBTB16 | 135 | 149 | 145 | 171 | 134 | 120 |

Dorsal spinal cord progenitor cell genes

| | | | | | | |
|---|---|---|---|---|---|---|
| TFAP2A | 67 | 61 | 84 | 43 | 100 | 166 |
| PAX3 | 112 | 99 | 180 | 84 | 193 | 158 |
| PAX7 | 156 | 106 | 186 | 100 | 148 | 209 |

Ventral spinal cord progenitor cell genes

| | | | | | | |
|---|---|---|---|---|---|---|
| OLIG2 | 1 | 1 | 1 | 1 | 1 | 1 |
| NKX2-2 | 1 | 1 | 1 | 1 | 1 | 1 |

TABLE 9
Inclusion and Exclusion Criteria

Inclusion Criteria
Participants were [symbol] for the study if all of [symbol] following inclusion criteria were met prior to dosing of LCTOPC1 [symbol]
1. Neurologically complete, traumatic SC [symbol] (American Spinal injury Assoc [symbol] Impairment Scale A) [symbol] motor or sensory function [symbol]
below the relevant sensory or motor (right or left) level.
2. Single neurological level from [symbol] through [symbol]
3. From [symbol] through [symbol] 5 years of age at [symbol] of injury.
4. Single spinal cord lesion on a post-stabilization MRI scan, with sufficient visualization of the spinal cord for 30 mm above and below the injury
epicenter to enable [symbol] -injection safety monitoring.
5. [symbol] nformed [symbol] sent for [symbol] protocol and the long-term follow-up protocol provided and documented (i.e., signed informed consent forms) no later [symbol]
11 days following injury.
6. Able to participate in elective surgical procedure to [symbol] ject LCTOPC1 7 to 14 days following [symbol] .
Exclusion Criteria
Participants were not eligible for the [symbol] if any of the following exclusion criteria were [symbol] prior to dosing of LCTOPC1:
1. Spinal cord injury due to penetrating trauma.
2. Traumatic anatomical transection or laceration of the spinal cord based on prior surgery or MRI.
3. Spinal cord lesions with anteroposterior diameter of the spinal cord <2 mm at point of maximal compression [symbol] image from a
post-stabilization MRI.
4. Any conc [symbol] injury that could interfere with the performance, interpretation or valididty of neurological examinations, such as multiple spinal
cord lesions, lumbar [symbol] injury, [symbol] injury, or traumatic brain injury.
5. Any treatment or pre-existing condition that could interfere with the performance, interpr [symbol] or validity of neurological examinations, such as
polyneuropathy, [symbol] or multi-focal neuropathy, [symbol] or [symbol]
6. [symbol] nability to communucate effectively with neurological examiner such that the validity of patient data could have been compromised.
7. Significant organ damage or systemic disease that would have created an unacceptable risk for surgery or [symbol] nosuppressio [symbol] .
8. Concomitant use at baseline of other immunosuppressive agents, such as corticosteriod [symbol] that would have created an unacceptable risk for [symbol]
immunosuppression with [symbol] .
9. Need for mechanical support of [symbol] ( [symbol] , contin [symbol] p [symbol] sitive airway pressure [symbol] bi-level positive airway pressure), excluding
supplemental oxygen, at baseline.
10. History of any malignancy.
11. Pregnant or nursing women. Female participants of childbearing potential agreed to prevent pregnancy by the use of contraception for 365 days
following LCTOPC1 injection [symbol] male participants agreed to use contraception to prevent pregnancy in any female partners of child bearing potential
for 365 days following LCTOPC1 injection.
12. Positive blood test for antibodies to human immunodeficiency virus 1 or 2 [symbol] ant [symbol] to hepatitis B virus [symbol] ore antigen [symbol] or antibodies to
hepititis C virus.
13. Pa [symbol] reactive antibodies (PRA) [symbol] 20% [symbol] if a site lab reported PRA only for human leukocyte antigen (HLA) Class [symbol] or seperately for HLA
Class [symbol] and [symbol] , exclusion was based solely on the [symbol] RA for HLA Class [symbol] .
14. Serum crea [symbol] above the established limit for the normal range [symbol] individual study center laboratories at base [symbol] .
15. Liver function tests >2 × the established upper limit for the normal range [symbol] individual study center laboratories at baseline.
16. Hematocrit ≤27% at baseline.
17. Positive blood cultures (48-hour culture results at Day- [symbol]
18. Active untreated viral, fungal or bacterial infection at baseline.
19. Evidence of surgical site infection at intended LCTOPC1 injection site at baseline.
20. Temperature [symbol] C. at 2 time points from Day-1 through Day 0 prior [symbol] surgery for LCTOPC1 injection.
21. Body mass index >35 [symbol] /m [symbol] or weight >300 pounds.
22. Active participation in another experimental procedure/intervention.
23. Psychoactive substance use disorder (as defined by the Diagnostic and Statistical Manual of Mental Disorders, Fourth Edition) as any time during
the 3 months preceding study entry.
24. History of major depression, schizophrenia, parania, or other psychotic disorders as defined by Diagnostic and Statistical Manual of Mental
Disorders, Fourth Edition.
25. Participant who, in the opinion of the investigator, was unlikely to return for all follow-up visits as specified in the protocol.
26. Any condition that, in the judgment of the investigator, woulld have precluded successful participation in the study. [symbol]
indicates data missing or illegible when filed

TABLE 10
Schedule of Events from Screening to Day 90

| [symbol] | [symbol] | [symbol] | [symbol] | [symbol] | [symbol] | [symbol] | | | | | | | | |
|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | | | | | | | | | | | | | |
| [symbol] | [symbol] | | | | | | | | | | | | | |
| [symbol] | [symbol] | | [symbol] | | | [symbol] | | [symbol] | | [symbol] | | [symbol] | | [symbol] |
| [symbol] | [symbol] | | [symbol] | [symbol] | [symbol] | [symbol] | | [symbol] | | [symbol] | | [symbol] | | [symbol] |
| [symbol] | | | [symbol] | | | | | [symbol] | | [symbol] | | [symbol] | | [symbol] |
| [symbol] | [symbol] | | [symbol] | [symbol] | | | | [symbol] | | [symbol] | [symbol] | [symbol] | [symbol] | [symbol] |
| [symbol] | | | [symbol] | | | | | [symbol] | | [symbol] | | [symbol] | | [symbol] |
| [symbol] | | | | | | | | | | [symbol] | | | | [symbol] |
| [symbol] | | | | | | | | | | [symbol] | | | | |
| [symbol] | [symbol] | | | | | | | [symbol] | | | | | | |
| [symbol] | [symbol] | | | | | | | [symbol] | | [symbol] | | [symbol] | | [symbol] |
| [symbol] | [symbol] | | | | | | | | | | | | | |
| [symbol] | [symbol] | | | | | | | | | | | | | |
| [symbol] | [symbol] | | [symbol] | | | [symbol] | | [symbol] | | [symbol] | | [symbol] | | [symbol] |
| [symbol] | [symbol] | | [symbol] | | | [symbol] | | [symbol] | | [symbol] | | [symbol] | | [symbol] |
| [symbol] | | | | | | | [symbol] | | [symbol] | | [symbol] | [symbol] | | |
| [symbol] | | | | | | | | | [symbol] | | [symbol] | [symbol] | | |
| [symbol] | | [symbol] | | | | | | | | | | | | |
| [symbol] | | | [symbol] | | | | | | | | | | | |
| [symbol] | | | [symbol] | | | | | | | [symbol] | | [symbol] | | |
| [symbol] | | | | | | | [symbol] | | [symbol] | | [symbol] | [symbol] | | |
| [symbol] | | | [symbol] | | | [symbol] | | [symbol] | | [symbol] | [symbol] | [symbol] | [symbol] | [symbol] |
| [symbol] | | | [symbol] | | | | | | | [symbol] | | | | |
| [symbol] | | | | [symbol] | | | | | | | | | | |
| [symbol] | | | | | | [symbol] | | | | | | | | |
| [symbol] | | | | | [symbol] | | | | | | | | | |
| [symbol] | | | | | | | | | | | [symbol] | | | |
| [symbol] | | | | | | | | | | | | [symbol] | | |
| [symbol] | | | | | [symbol] | | | | | | | [symbol] | | |
| [symbol] | | | | | [symbol] | | | | | | | | | |
| [symbol] | [symbol] | | [symbol] | [symbol] | [symbol] | [symbol] | | [symbol] | | [symbol] | | [symbol] | | [symbol] |
| [symbol] | [symbol] | | [symbol] | [symbol] | [symbol] | [symbol] | | [symbol] | | [symbol] | | [symbol] | | [symbol] |

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

TABLE 11
Schedule of Events from Day 120 to Day 365.

| | | | | Final |
|---|---|---|---|---|
| | Follow-up | | | Visit1 |
| | Day (window): | | | |
| | 120 | 180 | 270 | 365 |
| Procedures | (±7) | (±14) | (±14) | (±14) |
| Informed consent for companion | | | | X |
| long-term, follow-up study | | | | |
| Physical examination | X2 | X2 | X2 | X3 |
| Vital signs | X | X | X | X |
| Neurological examination | X | X | X | X |
| ISNCSCI examination | X | X | X | X |
| UA [symbol] -IM [symbol] | X | X | X | X |
| Pain questionnaire | | X | | X |
| Bowel and bladder questionnaire | | X | | X |
| SCIM | | X4 | | X4 |
| MRI | X5 | X6 | X5 | X6 |
| Hematology | X | X | X | X |
| Blood chemistry - panel 17 | X | X | X | X |
| Blood samples for immune | X | X | X | X |
| response (shipped) | | | | |
| Blood samples for | | | | X |
| xenotransplantation (shipped) | | | | |
| Concomitant medications | X | X | X | X |
| Adverse events | X | X | X | X |

ALT = alanine aminotransferase;
AST = aspartate aminotransferase;
Gd-DTPA = gadolinium diethylenetriamine pentaacetic
1If a participant's participation terminated between Day 60 and Day 365, all tests and procedures scheduled for the Da [symbol]
2Brief examination.
3Complete exam
4SCIM was collected as a questionnaire (versus observation of activities).
5MRI included the spinal cord and cerebellum, with and without Gd-DTPA contrast.
6MRI included the brain in addition to the routine imaging of the spinal cord and cerebellum, with and without Gd-DTP.
7Serum albumin, alkaline phosphatase, blood urea nitrogen, total bilirubin, chloride, serum creatinine, glucose, potassi [symbol] [symbol]
indicates data missing or illegible when filed

Example 9—a Phase 1/2a Dose Escalation Study of AST-OPC1 in Subjects with Subacute Cervical Spinal Cord Injury

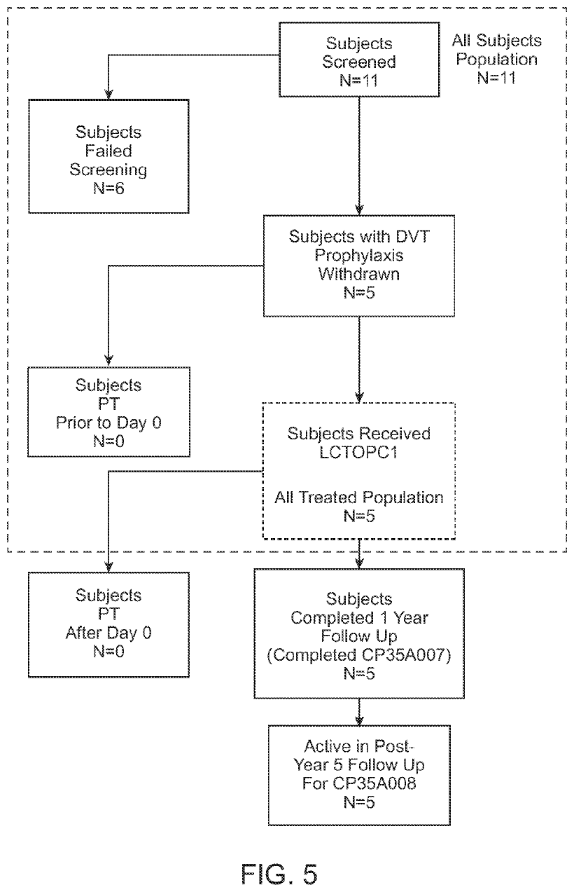

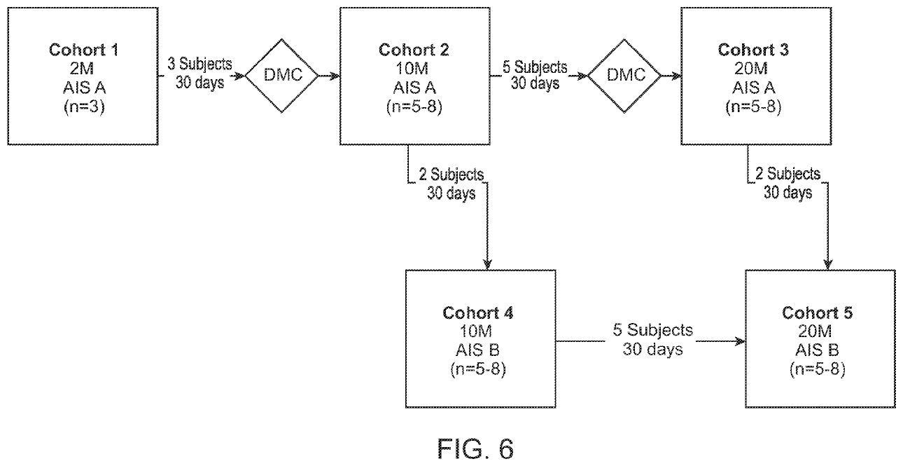

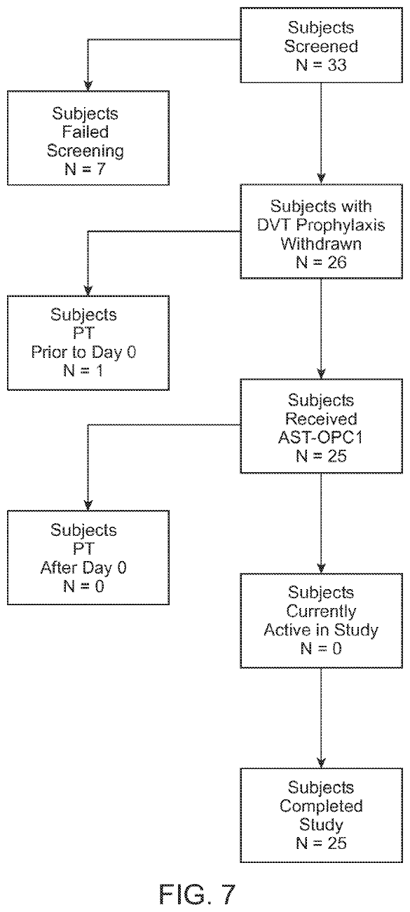

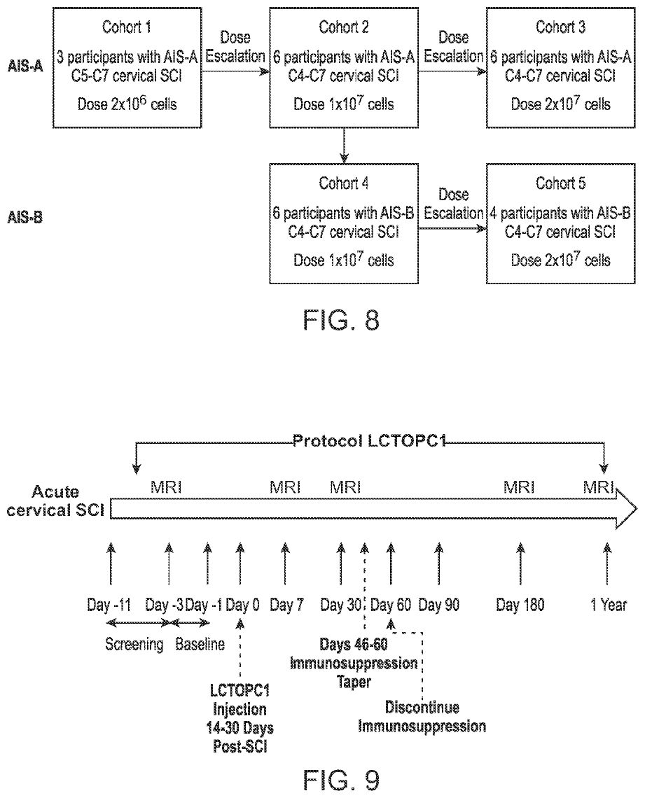

Study Design. The trial design was an open-label, staggered dose escalation, cross-sequential, multicenter study. Three sequential, escalating doses of AST-OPC1 were administered at a single tie point between 21 and 42 days post-injury to subjects with subacute cervical spinal cord injuries (SCI). Subjects received AST-OPC1 via direct intra-spinal injection using a Syringe Positioning Device (SPD). To prevent rejection of engraftment, low dose tacrolimus was initiated 6 to 12 hours after and intra-spinal injection, and continued for 46 days, tapered from Day 46 to Day 60, and was discontinued at Day 60. Subjects were followed for 1 year following administration of AST-OPC1 under this protocol. Male or female subjects from 18 to 69 years of age at time of consent with sensorimotor complete, traumatic SCI (American Spinal Injury Association Impairment Scale A) or sensorimotor incomplete, traumatic SCI (American Spinal Injury Association Impairment Scale B). Subjects had a single neurological level of injury (NLI) from C-5 through C-7 or a C4 NLI with an upper extremity motor score (HEMS)≥1. There was a single spinal cord lesion on a post-stabilization magnetic resonance imaging (MRI) scan, with sufficient visualization of the spinal cord injury epicenter and lesion margins to enable post-injection safety monitoring. Subjects were able to participate in an elective surgical procedure to inject AST-OPC1 21 to 42 days following SCI. Subjects received a single dose of either 2×10⁶, 1×10⁷, or 2×10⁷ AST-OPC1 viable cells by injection, administered 21 to 42 days following SCI. The product was delivered intraoperatively into the spinal cord using the Syringe Positioning Device. The AST-OPC1 batch numbers used in this study were M08D1A, M22D1A and M25D1A. Single administration of AST-OPC1 was provided with 1 year of follow-up. Schematics of the planned study timeline, and subject screening and treatment, are provided in FIG. 6 and FIG. 7, respectively. A schematic diagram of the final open-label, staggered dose-escalation, multicenter Phase 1/2a clinical trial is shown in FIG. 8. An overview of study visits for the one-year protocol is presented in the study schema in FIG. 9.

A neurological examination was completed using the standardized International Standards for Neurological Classification of Spinal Cord Injury (ISNCSCI) examination for motor and sensory testing and for designation of the American Spinal Injury Association impairment scale. The ISNCSCI is used for efficacy with respect to improved motor function in the extremities, improved sensory function, and/or a descending neurological level. Safety assessments included physical examination, vital signs, ISNCSCI neurological examination, pain questionnaire (International Spinal Cord Injury Pain Basic Data Set, ISCIPBDS), electrocardiogram (ECG), Magnetic Resonance Imaging (MRI), laboratory tests, concomitant medications, and adverse events (AEs). Results for the ISNCSCI were listed for each subject by scheduled visit and analyzed by change in motor level and motor scores as well as other exploratory assessments of arm/hand function, self-care ability and overall volitional performance. Adverse events were tabulated by system organ class and by preferred term within system organ class according to the Medical Dictionary for Regulatory Activities (MedDRA®) Version 18.0. Statistical analysis of the safety data was performed using descriptive statistical methods including AEs incidence, severity and relatedness to AST-OPC1, to the injection procedure for product administration, and concomitant immunosuppression with tacrolimus. The number of laboratory assessments (hematology, clinical chemistry) that were below, within, or above the normal laboratory reference range were summarized for each analyte at scheduled study visits. Vital signs were summarized by calculating the mean, standard deviation, median, and range of values at each of the protocol-specified time points.

Results. The study enrolled 26 subjects. Twenty-five subjects were administered AST-OPC1 at 5 study sites. All 25 subjects completed 1 year of follow-up. The 25 subjects who were administered AST-OPC1 ranged in age from 18 to 62 years, 21 subjects were male and 4 were female, and the majority were Caucasian (22 subjects). Vehicular accident was the cause of SCI in 8 subjects. None of 25 treated subjects exhibited evidence of unexpected neurological deterioration on ISNCSCI examinations after completing 1-year follow-up. The safety data indicates that AST-OPC1 can be safely administered to subjects in the subacute period after cervical SCI. The injection procedure and the low-dose temporary immunosuppression regimen were well tolerated. The 25 subjects who received AST-OPC1 completed 1 year of follow-up and showed no evidence of neurological deterioration or adverse findings on MRI scans.

Table 12 below shows the AST-OPC1 dose cohorts and injection preparations used in the study. AST-OPC1 is a cryopreserved cell population containing a mixture of oligodendrocyte progenitor cells and other characterized cell types that are obtained following differentiation of undifferentiated human embryonic stem cells. At the time of cryopreservation, each vial contained 7.5×10⁶ viable cells in 1.2 mL of cryopreservation medium. The components of the cryopreservation medium were the following: Glial Progenitor Medium (GPM)—86% (v/v) [98% DMEM/F12 with GlutaMAX supplement, 1.9% B-27 supplement and 0.1% T3]; 25% Human serum albumin (HSA)—3.6% (v/v); 1 M HEPES—0.9% (v/v); DMSO—9.5% (v/v).

TABLE 12
Dose Cohorts and Injection Preparation.

| | | | Volume | | |
|---|---|---|---|---|---|
| | Dose | Concen- | per | | Total Volume |
| | (No. of | tration | Injection | Number of | Administered |
| Cohort | cells) | (cells/μL) | (μL) | Injections | (μL) |
| 1 | 2 × 10⁶ | 4 × 10⁴ | 50 | 1 | 50 |
| 2 | 1 × 10⁷ | 2 × 10⁵ | 50 | 1 | 50 |
| 3 | 2 × 10⁷ | 2 × 10⁵ | 50 | 2 | 100 |
| 4 | 1 × 10⁷ | 2 × 10⁵ | 50 | 1 | 50 |
| 5 | 2 × 10⁷ | 2 × 10⁵ | 50 | 2 | 100 |

Dose selection and Timing. The first proposed dose of 2×10⁶ cells was evaluated for safety in a previous thoracic SCI trial. 2×10⁶ cells were used again in Cohort 1 in this trial to establish lack of complications due to the injection procedure. The increase from the first dose (2×10⁶ cells in 50 μL) to the second dose (1×10⁷ cells in 50 μL) only entails increasing the concentration of AST-OPC1 such that both of these doses were delivered via a single injection with a 50 μL volume. Therefore, the neurosurgeons consulted for this study viewed this initial dose escalation as a very small step with respect to the potential risks associated with the injection procedure. In addition, the safety of administering the second dose (1×10⁷ cells in 50 μL) was demonstrated in the uninjured pig cervical spinal cord at C6. This study confirmed the minimal expected tissue damage associated with injections into the uninjured spinal cord, and no evidence of efflux or cellular dissemination via the CSF was observed. The third dose represents an additional injection of 1×10⁷ cells in 50 μL at a second site within the lesion in a manner similar to that used for the rodent safety studies. This dose is within the 6 to 12× safety margin relative to the highest dose tested in the rat safety studies, particularly with respect to the total volume injected.

LCTOPC1 was supplied to the clinical sites in sterile, single-dose, single-use, 2.0 mL Corning™ cryovials. At the time of cryopreservation, each vial typically contained 7.5×10⁶ viable cells in 1.2 mL of cryopreservation medium. The components of the cryopreservation medium were the following: 1) Glial Progenitor Medium (GPM)—86% (v/v) [98% DMEM/F12 with GlutaMAX supplement, 1.9% B-27 supplement and 0.1% T3]; 2) 25% Human serum albumin (HSA)—3.6% (v/v); 3) 1 M HEPES—0.9% (v/v); 4) DMSO—9.5% (v/v). The cryopreserved drug product was thawed, washed, resuspended in the injection medium, and loaded into the injection syringe at the clinical sites.

LCTOPC1 is a cell population containing a mixture of oligodendrocyte progenitor cells (OPCs) and other characterized cell types that are obtained following differentiation of undifferentiated human embryonic stem cells (uhESCs). LCTOPC1 Drug Product (DP) is manufactured by a continuous process. Harvested LCTOPC1 Drug Substance is a transient intermediate that is immediately formulated, vialed, and cryopreserved to LCTOPC1 DP without the use of a hold step. Compositional analysis of LCTOPC1 by immunocytochemistry (ICC), flow cytometry, and quantitative polymerase chain reaction (qPCR) indicates that the cell population is comprised primarily of neural lineage cells of the oligodendrocyte progenitor phenotype. Other neural lineage cells, namely astrocytes and neurons, are present at low frequencies. The only non-neural cells detected in the population are epithelial cells. Mesodermal and endodermal lineage cells, and uhESCs are routinely below the quantitation or detection limits of the assays.

It is hypothesized that the subacute phase of SCI is the optimal time window in which to administer AST-OPC1. This phase avoids the early damage that leads to apoptosis of endogenous oligodendrocytes and occurs soon enough to allow the AST-OPC1 cells to migrate to denuded axons before extensive glial scarring has occurred. This hypothesis is supported by studies in rodent models of SCI that have shown functional benefits when AST-OPC1 or other similar preparations are injected 7 days after SCI, but no benefit if the interval between injury and injection is greater than 8 weeks (Keirstead 2005, Karimi-Abdolrezaee 2006). Since spontaneous functional recovery in rats with contusion SCI begins to plateau at about 6 weeks post-injury, injection of AST-OPC1 at 7 days corresponds to about ⅙ (17%) of the time elapsed between injury and the onset of recovery plateau.

The subacute phase of SCI is thought to be much longer in humans given that the rate of spontaneous recovery typically begins to plateau at about 6 months after SCI (Fawcett 2007). Extrapolating from the nonclinical efficacy data in rodents, injection of AST-OPC1 when one-sixth of the time to onset of recovery plateau has elapsed in humans would correspond to about 30 days post-injury.

The original dosing window of 14 to 30 days was selected to avoid the early hemorrhage and inflammation that occurs following SCI, as well as the scar tissue formation that occurs in the chronic phase of SCI. This window was also based on the preclinical data available prior to the initiation of this clinical study. However, a dedicated preclinical study was performed which suggested that the optimal dosing window in human subjects may extend to a maximum 60 days post-SCI.

Review of the new preclinical study data was conducted by a panel including study investigators, and several SCI experts to determine whether the dosing window should be adjusted. Additionally, during the review, consideration was given to the planned inclusion of subjects with a C4 NLI. The final recommendations indicated that a dosing window of 21 to 42 days post-SCI would still be considered within the subacute period, while allowing more time for subjects to be medically stable prior to undergoing elective surgery for AST-OPC1 injection. Therefore, AST-OPC1 was administered to subjects in this study at 21 to 42 days after SCI.

Tacrolimus management. Immunosuppression with tacrolimus was initiated between 6 and 12 hours after injection of AST-OPC1. If the subject was unable to take oral medication, tacrolimus was administered intravenously at a starting dose of 0.01 mg/kg/day, given as a continuous intravenous infusion. Subjects were switched to oral tacrolimus as soon as they were able to take medication by mouth. The starting dose for oral tacrolimus was 0.03 mg/kg/day, divided into 2 daily doses. The tacrolimus dose was adjusted to achieve a target whole blood trough level of 3 to 7 ng/ml. This target range was slightly below the typical range for long-term maintenance therapy following solid organ transplantation and was selected based on the low allogenic reactogenicity of AST-OPC1. At Day 46, the tacrolimus dose was decreased by 50% (rounded to the nearest 0.5 mg, since this was the smallest capsule size available). At Day 53, the tacrolimus dose was decreased by another 50% (rounded to the nearest 0.5 mg). If the rounded total daily dose was 0.5 mg or lower, the subject received 0.5 mg once per day until tacrolimus was discontinued. If the rounded total daily dose was 0.5 mg or lower, the participant received 0.5 mg once per day until tacrolimus was discontinued. Tacrolimus was discontinued at Day 60 (FIG. 2). The dose of tacrolimus was lowered if the trough blood level exceeded 7 ng/mL. In addition, an expert reviewed all ISNCSCI examination forms to assess whether there were any changes in neurological function that may have been associated with tacrolimus tapering and/or discontinuation. Tacrolimus was discontinued if any of the following occurred: infection, uncontrolled fever, liver function test elevation, serum creatinine elevation, seizure, or tacrolimus-induced thrombotic thrombocytopenic purpura. Tacrolimus was discontinued at Day 60.

A schedule of the evaluations and procedures that were to be performed from screening to day 365 is provided in Table 13 below.

TABLE 13
Schedule of Events
Part A

| | Screen | Baseline | Surgery | Post-Injection | | |
|---|---|---|---|---|---|---|
| | Days −11 | Days −2 | Injection | Days | Day 7 | Days |
| Procedure | to −3 | to −1 | Day | 1-6 | (+/−1 day) | 8-29 |
| Demographic data | X | | | | | |
| Past and current medical history | X | | | | | |
| Complete physical exam | X | | | | | |
| Brief physical exam | | X | | Day 1 | X | |
| Vital signs | X | X³ | X | Daily | X | |
| Neurological exam | | X⁵ | | | X | |
| ISNCSCI exam | X | X¹ | | | X | |
| GRASSP | | X⁵ | | | | |
| SCIM | | X⁵ | | | | |
| MRI² | Day −7 to −3 | | | | X | |
| ECG | X | | | | X | |
| Hematology | X | X | | Day 1 | X | |
| Blood chemistry | X | X | | Daily | X | 2/week |
| Serology for HIV, HBV, HCV | X | | | | | |
| Panel reactive antibodies | X | | | | | |
| Pregnancy test, if applicable | X | | | | | |
| 48-hour blood culture | Day −3 | | | | | |
| Fasting blood glucose | | X⁴ | | | | |
| Blood for HLA typing | | X⁵ | | | | |
| Blood for immune response monitoring | | X⁵ | | | X | |
| Blood for xenotransplantation archival | | X⁵ | | | | |
| Withhold DVT prophylaxis | | Day −1 | | | | |
| CSF via lumbar puncture | | | X | | | |
| Begin tacrolimus | | | X | | | |
| Restart DVT prophylaxis | | | | Day 1 | | |
| Tacrolimus blood levels | | | | Day 3⁶ | X | 2/week |

| | |
|---|---|
| Concomitant medications | → |
| Adverse events | → |

Part B

| | Day 30 | | Day 60 | Day 90 | Day 180 | Day 270 | Day 365 |
|---|---|---|---|---|---|---|---|
| Procedure | (+/−3 days) | Days 31-59 | (+/−7 days) | (+/−7 days) | (+/−14 days) | (+/−14 days) | (+/−14 days) |
| Complete physical exam | | | | | | | X |
| Brief physical exam | X | | X | X | X | | |
| Vital signs | X | | X | X | X | | X |
| Neurological exam | X | | X | X | X | | X |
| ISNCSCI exam | X | | X | X | X | X | X |
| GRASSP | | | | X | X | X | X |
| SCIM questionnaire | X | | X | X | X | X | X |
| Pain questionnaire | X | | | X | X | | X |
| Bowel/Bladder questionnaire | X | | | | X | | X |
| MRI² | X | | | | X | | X |
| Hematology | X | | X | X | X | | X |
| Blood chemistry | X | 1/week | X | X | X | | X |
| Fasting blood glucose | X | | | | | | |
| Blood for immune response monitoring | X | | X | X | X | | X |
| Tacrolimus blood level | X | 1/week | X | | | | |
| CSF via lumbar puncture | | | X | | | | |
| Blood for xenotransplatation archival | X | | | | | | X |

| | |
|---|---|
| Concomitant medications | → |
| Adverse events | → |

Abbreviations: CSF, cerebrospinal fluid; GRASSP, Graded Redefined Assessment of Strength, Sensibility and Prehension, ISNCSCI, International Standards for Neurologic Classification of Spinal Cord Injury; MRI, magnetic resonance imaging; SCIM, Spinal Cord Independence Measure.
¹ISNCSCI exam (may be performed on Day −3 to −1, unless the screening ISNCSCI was performed on Day −3)
²MRI of cervical spine and brain at Screen & Day 365: MRI of cervical spine only at Days 7, 30, 180
³Vitals on day −2 and −1
⁴May be done pre-op on Injection Day
⁵May be performed as early as Day −4 if required to accommodate clinical site staff availability
⁶Tacrolimus blood level on Day 3 may be obtamed +/−1 day

Efficacy Evaluations. Neurological examinations were performed using the standardized International Standards for Neurological Classification of Spinal Cord Injury (ISNCSCI) examination for motor and sensory testing and for designation of the American Spinal Injury Association impairment scale. Upper Extremity Motor Score (0-50) and change from baseline were summarized with N, mean, standard deviation, 95% confidence interval, median, minimum, and maximum for the overall Intent to Treat population. A positive change is considered improvement and a negative change is considered worsening.

Motor and sensory level at each visit and changes in motor/sensory level from baseline were defined as follows. “Change in Level” is defined as the number of levels the motor/sensory level has changed from baseline. A positive number represents a change in the caudal direction; this is considered improvement. A negative number represents a change in the rostral direction; this is considered worsening.

The change in motor and sensory level was tabulated as follows: Percentage of subjects with an ascending motor level on either side of the body relative to baseline; Percentage of subjects with no motor level change on either side of the body relative to baseline; Percentage of subjects with one motor level improvement on at least one side of the body relative to baseline; Percentage of subjects with one motor level improvement on both sides of the body relative to baseline; Percentage of subjects with a two or more motor level improvement on at least one side of the body relative to baseline.

Efficacy Results. Efficacy for this study was measured by the change in ISNCSCI exam upper extremity motor score (UEMS) and change in motor level from baseline to 12 months after injection of AST-OPC1. A total of 22 subjects were part of the intent to treat population (Cohorts 2-5). The mean UEMS for the 22 subjects who completed the Day 365 visit was 28.4 (min: 7, max: 46), with a mean change from baseline of 8.9 points. There were three subjects (2004, 2007, 2008) who presented with some improvement in lower extremity motor scores (LEMS) without correlation with the level of improvement in UEMS. A total of 7 (31.8%) out of 22 subjects attained a two motor level improvement on at least one side of the body at the Day 365 visit and 21 (95.5%) subjects out of 22 subjects attained a one motor level improvement on at least one side at the Day 365 visit relative to Baseline. The percentage of subjects with motor level improvement is shown in Table 14 below.

TABLE 14
Percent of Motor Level Improvement from Baseline.

| | ≥2 Motor Levels | | ≥1 Motor Level | | ≥1 Motor Level | | | Ascending | |
|---|---|---|---|---|---|---|---|---|---|
| Visit | At least one side | | On both sides | | At least one side | No change | | Motor Level | |
| (N) | % (n) | | % (n) | | % (n) | % (n) | | % (n) | |
| Day 7 (22) | 0 | (0) | 0 | (0) | 45.5 (10) | 54.5 | (12) | 0 | (0) |
| Day 30 (22) | 9.1 | (2) | 18.2 | (4) | 45.5 (10) | 54.5 | (12) | 0 | (0) |
| Day 60 (21) | 14.3 | (3) | 28.6 | (6) | 61.9 (13) | 38.1 | (8) | 0 | (0) |
| Day 90 (22) | 18.2 | (4) | 36.4 | (8) | 81.8 (18) | 18.2 | (4) | 0 | (0) |
| Day 180 (22) | 13.6 | (3) | 54.5 | (12) | 86.4 (19) | 13.6 | (3) | 0 | (0) |
| Day 270 (20) | 25.0 | (5) | 45.0 | (9) | 85.0 (17) | 10.0 | (2) | 5.0 | (1) |
| Day 365 (22) | 31.8 | (7) | 59.1 | (13) | 95.5 (21) | 4.5 | (1) | 4.5 | (1) |

Study Endpoints. The primary endpoint of the trial was safety, as measured by the frequency and severity of adverse events (AEs) and serious adverse events (SAEs) within 1 year of LCTOPC1 injection that were related to LCTOPC1, the injection procedure used to administer LCTOPC1, and/or the concomitant immunosuppression administration. Measurements to assess safety included physical exams, vital signs, electrocardiogram (ECG), neurological exams, ISNCSCI exams, magnetic resonance imaging (MRI) scans, pain questionnaire, concomitant medications, AEs, and laboratory tests for hematology, blood chemistry, and immunosuppression safety monitoring. The secondary endpoint was neurological function as measured by the International Standard for Classification of Spinal Cord Injury (ISNCSCI). The ISNCSCI is a highly reproducible research and clinical assessment of neurological impairment for individuals with SCI and has been used as a tool to evaluate the effectiveness of acute SCI clinical interventions (Rupp PMID: 34108832; Marino 2008 PMID: 18581663). To maximize the inter-rater and intra-rater reliability of neurological assessments, a half-day training session, led by an external expert and including examinations of individuals with SCI, was required as part of each center's site initiation visit. During the first year of the study, ISNCSCI examinations were performed at 30, 60, 90, 180, 270, and 365 days after injection of LCTOPC1. The 365-day follow-up visit was pre-specified as the time point for the secondary endpoint.

STATISTICAL ANALYSIS FOR EFFICACY. The efficacy endpoint, neurological function, was evaluated by characterizing upper extremity motor scores and motor level on the ISNCSCI examination (point estimate and 95% confidence interval) by time point at 30, 60, 90, 180, 270, and 365-days post-injection of LCTOPC1. The baseline for the ISNCSCI assessment was defined as the Baseline Visit performed between 24 to 48 hours prior to injection. Upper Extremity Motor Score and change from baseline was summarized by participant, mean, standard deviation, 95% confidence interval, median, minimum, and maximum for the overall intent-to-treat population.

STATISTICAL ANALYSIS FOR SAFETY. The collection period for adverse events (AEs) began once the participant had signed the informed consent form and ended after 365 days of observation. Statistical analysis of AEs started on or after the date and time of the LCTOPC1 injection, or an AE that started before the LCTOPC1 injection, and worsened after the administration of the investigational product. AEs were tabulated by system organ class (SOC) and by preferred term (PT) within system organ class, according to the Medical Dictionary for Regulatory Activities (MedDRA®) Version 18 and reported by participants. A topline summary of AEs with the number of events, number of participants, and percentage of participants for each category was tabulated by cohort and overall. Categories for possible relationship included: LCTOPC1, injection procedure, and tacrolimus. Tabulations were prepared for all AEs, related events, Grade 3 and higher events, and serious events.

TABLE 15
Summary of Related Adverse Events in All Treated Participants

| | AE | SAE | | Days from | | |
|---|---|---|---|---|---|---|
| Preferred Term | (N) | (N) | Causality | Injection | Outcome | Severity |
| Dysesthesia | 1 | None | OPC1 | 47 | Resolved* | Grade 2 |
| Cerebrospinal Fluid | 2 | 1 | Injection | 11-15 | Resolved with | Grade 2-3 |
| Leakage | | | procedure | | sequelae** | |
| Cerebrospinal fluid | 5 | None | Injection | 6-7 | Resolved | Grade 1 |
| retention | | | procedure | | | |
| Pain (neck, muscular, | 5 | None | Injection | 0-1 | Resolved | Grade 1-3 |
| incisional) | | | procedure | | | |
| Headache | 3 | None | Injection | 0-84 | Resolved | Grade 1-2 |
| | | | procedure | | | |
| Nausea | 1 | None | Injection | 0 | Resolved | Grade 2 |
| | | | procedure | | | |
| Postoperative wound | 1 | None | Injection | 7 | Resolved | Grade 1 |
| infection | | | procedure | | | |
| Vomiting | 1 | None | Injection | 0 | Resolved | Grade 2 |
| | | | procedure | | | |
| Autonomic Dysreflexia | 1 | None | Injection | 0 | Resolved | Grade 3 |
| | | | procedure | | | |
| Incisional drainage | 1 | None | Injection | 1 | Resolved | Grade 1 |
| | | | procedure | | | |
| Hypomagnesemia | 8 | None | Tacrolimus | 5-43 | Resolved | Grade 1-2 |
| Bacterial infection | 1 | 1 | Tacrolimus | 30 | Resolved | Grade 3 |
| Decreased Appetite | 1 | None | Tacrolimus | NK | Resolved | Grade 2 |
| Nausea | 1 | None | Tacrolimus | 7 | Resolved | Grade 2 |

Abbreviations: NK, not known; AE, adverse event; SAE, serious adverse event.
*Dysesthesia resolved at Year 2 follow-up.
**One Cerebrospinal Fluid Leakage required lumbar drainage. Participant returned to Rehabilitation on Day 22.

DISCUSSION The safety data from this study suggest that AST-OPC1 can be safely administered to participants in the subacute period after cervical SCI. The injection procedure and the low-dose temporary immunosuppression regimen were well tolerated. None of the 25 participants who received LCTOPC1 showed evidence of neurological deterioration. There were no SAEs reported as directly related to LCTOPC1 and evaluation of the AEs did not show an increase in incidence for commonly reported SCI complications, such as urinary tract infections, muscle spasms, or neuropathic pain (Sezer 2015). In this study involving participants with cervical C4-C7 AIS-A and AIS-B, at one-year follow-up, 24/25 (960%) of participants recovered one or more levels of motor function on at least one side of their body and 8/25 (32%) of participants recovered two or more levels of motor function on at least one side of their body. Improvement of two motor levels can change a person's functional capacity from requiring total assistance for activities of daily living to near independence (Whiteneck et al. 1999). The safety and neurological recovery data, from both the thoracic and cervical trials, have provided evidence that hESC-derived treatments can be safely delivered into the spinal cord.

Example 10—Decorin Secretion as a Potency Assay for OPC1.

Treatment with recombinant Decorin in rat models of spinal cord injuries (SCI) has been shown to inhibit inflammation and glial scar formation and may promote axonal growth across the injury interface after acute spinal cord injury (Wu, Li et al, 2013, Ahmed, Bansal et al. 2014). Decorin has been shown to suppress acute scarring and wound cavitation and induce dissolution of mature scar tissue in dorsal funicular lesion SCI model system of the spinal cord in adult rats (Wu, Li et al, 2013, Ahmed, Bansal et al 2014). DFL cavity treatment with recombinant Decorin suppresses inflammation and scar deposition in the acute and subacute phases of the CNS injury response in rat model of SCI and also contributes to dissolution of the mature scar following SCI (Esmaeli, Berry et al 2014, Ahmed, Bansal et al 2014). OPC1 treatment in non-clinical models of SCI has demonstrated similar results to that seen in the published studies above.

OPC1 cells have been shown to produce large amounts of Decorin. The results seen in the OPC1 animal studies demonstrate very similar anatomical outcomes to that seen in the studies above. Thus, the anatomical effects observed in the nonclinical efficacy studies of OPC1 transplantation into SCI injury may be attributed, at least in part, to the secretion of Decorin.

While the preclinical studies to date have established the optimal window for OPC1 implantation to achieve maximum efficacy between 14-60 days post-injury, Decorin has been shown to have an effect in dissolving mature scars following SCI (Esmaeli, Berry et al 2014, Ahmed, Bansal et al 2014), lending evidence for a potential role of OPC1 cells in the treatment of chronic SCI subjects.

### Introduction

Decorin is a naturally occurring extracellular small leucine-rich proteoglycan TGF-β1/2 antagonist which regulates diverse cellular functions through interactions with components of the extracellular matrix (ECM) and plays several key roles in the cellular response to spinal cord injury. Accordingly, Decorin secretion in vitro was developed and qualified as a potency assay for OPC1.

Briefly, OPC1 Drug Product cells are thawed and cultured for 48 hours, then the media is collected and secreted Decorin concentration measured by an ELISA assay.

Preliminary molecular analysis and an initial ELISA revealed that Decorin is not secreted by H1 hESC, and that its expression is gradually turned on during OPC1 differentiation, with the highest levels of a secreted protein detected in the drug product. This, along with scientific literature showing a biological activity inclusive of scarring suppression at injury site and stimulation of axonal growth through spinal cord injury (SCI) site, supports Decorin as a suitable potency candidate.

Decorin (secretion) is useful as a potency indicator for OPC1 cells by describing its ability to modulate SCI tissue remodeling, via attenuating harmful processes.

Decorin is a biological modulator secreted by OPC1

OPC1 Drug Product is a cryopreserved cell population containing oligodendrocyte progenitor cells and other characterized cell types that are obtained following differentiation of H1 human embryonic stem cells (hESC). OPC1 has been shown to have three potentially reparative functions which address the complex pathologies observed at the SCI injury site. These activities of OPC1 include production of neurotrophic factors, stimulation of vascularization, and induction of remyelination of denuded axons, all of which are critical for survival, regrowth and conduction of nerve impulses through axons at the injury site. One of the potential routes by which OPC overcome the inhibitory factors at the injury site may be Decorin upregulation as a response of NG2⁺ (neuron-glial antigen 2, CSPG4) cells to retinoic acid (Goncalves, Wu et al. 2019).

It is important to note that Decorin secretion is acquired by OPC1 cells during the differentiation process as part of their maturation, feature enabling easy and quantifiable Decorin measurement as a possible potency marker of OPC1.

Decorin secretion was approximately 25 ng Decorin per day. Accordingly, the current threshold for the Decorin potency assay was defined as 25 ng/ml.

Decorin has been shown to be effective in reducing scarring, when produced endogenously by different cell types at the injury site or given exogenously in a recombinant form in non-clinical models of SCI, as described below.

Thus, active de-novo secretion of endogenous Decorin by OPC1 during its implantation into the SCI cavity may be a key component to ensure successful treatment, and, as such, may be a good potency marker.

Spinal Cord Injury

The majority of traumatic SCIs result in contusion or compression of the spinal cord. The mechanical insult (primary injury) in these cases causes a cascade of molecular and cellular changes that are collectively referred to as the secondary injury (Kakulas 1999). Some of the pathological changes associated with secondary injury include petechial hemorrhages progressing to hemorrhagic necrosis, free radical-induced lipid peroxidation, elevated intracellular calcium leading to activation of neutral proteases, accumulation of extracellular potassium, accumulation of excitatory amino acids, and ischemia (Anderson 1993, Hulsebosch 2002). Traumatic demyelination also begins within a few hours after injury (Kakulas 1999).

The cellular response to SCI is generally considered to consist of 3 phases: an acute hemorrhagic phase when hematogenous inflammatory cells invade the wound; a sub-acute phase when scarring is initiated from astrocytes interacting with invading meningeal fibroblasts to produce a glia limitans around the wound cavity with a core of extracellular matrix (ECM) proteins, revascularization is also initiated, and axon growth is arrested at the wound margins; and a consolidation or chronic phase when ECM deposits are remodeled by proteases to establish a mature contracted scar.

The superimposition of progressive wound cavitation on top of the cellular response results in a progressive cystic expansion of an astrocyte-free void filled with proteoglycans and macrophages and bordered by a proteoglycan-rich neurophils that cause secondary destruction of axons.

Cellular and Extracellular Composition of the Spinal Cord Injury Scar Tissue

Traumatic SCI triggers a complex cascade of events that culminates in the formation of a scar which consists of multiple cell types, as well as extracellular and non-neural components. In the acute post-injury phase (0-72 h), cell death and damage lead to release of a number of cellular and blood-derived damage associated molecular patterns (DAMPs). These are powerful activating and inflammatory stimuli for stromal cells, astrocytes, NG2+ OPCs and microglia. Fibroblast-like cells proliferate from perivascular origin in this acute phase. Activated cells increase deposition of ECM molecules such as Chondroitin sulfate proteoglycans (CSPGs) and stromal-derived matrix. Circulating immune-responders (neutrophils, monocytes) are recruited, their relative expression of cytokines, chemokines and matrix metalloproteinases is that of a mixed cell phenotype (pro-inflammatory and pro-resolving). Over time, the injury microenvironment becomes increasingly proinflammatory. In the chronic spinal injury scar, monocyte-derived macrophages/microglia adopt a predominantly pro-inflammatory phenotype. Rather than entering a resolution phase, responding innate immune cells present DAMPs to circulating adaptive immune cells and the pathology spreads. Hypertrophy of reactive astrocytes, upregulated expression of intermediate-filament associated proteins and secretion of matrix CSPGs occur. Scar-forming reactive astrocytes are organized into a barrier-like structure, which separates spared tissue from a central region of inflammation and fibrosis where wound-healing fails to undergo resolution. In most mammalian species, a chronic cystic cavity develops. Wallerian degeneration of injured axonal projections contributes to continued extracellular deposition of axonal and myelin debris, which is ineffectively processed by immune cells, and along with CSPGs, acts to inhibit neuronal regeneration and neuroplasticity (Bradbury and Burnside 2019).

Function of Decorin in Spinal Cord Injury

OPC1 clinical application is aimed at the sub-acute phase, 21-60 days post-SCI. It is thus assumed that the transplantation of OPC1 occurs during the transition from acute to chronic phase, in an inflammatory active environment. Hence, the ability of OPC1 to actively secrete Decorin that can potentially reduce the ongoing negative cues may be useful for its therapeutic activity.

Decorin suppresses CNS scarring through several mechanisms (Esmaeili, Berry et al. 2014, Gubbiotti, Vallet et al. 2016) including: (1) attenuating both TGF-β1/2 receptor activation and signaling through down-stream SMADs (a family of intracellular proteins that mediate signaling by members of the TGF-beta superfamily), that mediate transcriptional activation of ECM production; (2) binding to type I collagen fibrils to inhibit fibrogenesis; (3) forming an activity-blocking complex with connective tissue growth factor (CTGF); (4) binding to fibronectin and inhibiting cell adhesion and fibroblast migration; (5) abrogating inflammation, CSPG/laminin/fibronectin-rich scar formation and the injury responses of astrocytes, microglia and macrophages; (6) stimulating microglia to secrete plasminogen/plasmin (the activity of which is moderated by PAI-1), which then regulates matrix metalloproteinase (MMP): tissue inhibitors of MMP (TIMP) ratios in wounds to initiate fibrolytic degradation of ECM underpinning remodeling during the consolidation phase of acute scarring; and (7) binding to the epidermal growth factor receptor (EGFR), hepatocyte growth factor (Met) receptor and toll-like receptors to modulate angiogenesis and inflammatory responses.

Recombinant human Decorin (Galacorin), was investigated as a potential treatment for macular degeneration, diabetic retinopathy and diabetic macular edema (Nastase, Janicova et al. 2018). In the U.S. Pat. No. 9,061,047B2, the authors suggest using Galacorin for preventing or reducing scar formation by its administration to patients with neurological conditions including central nervous system injuries and/or diseases. A formulation of Decorin in an eye drop was reported as an anti-scarring agent that can replace corneal transplantation (Hill, Moakes et al. 2018).

Decorin promotes axon regeneration directly by suppressing the production of scar-derived growth inhibitory ligands (Esmaeili, Berry et al. 2014) and indirectly by: (1) plasmin activation of neurotrophins (Davies, Tang et al. 2006); (2) disinhibition of axon growth cone advance by digestion of CSPG and CNS myelin inhibitors through plasmin and plasmin induced activation of MMP (Minor, Tang et al. 2008); and (3) suppression of EGFR activity in growth cones, thereby potentially blocking CSPG/CNS myelin mediated growth cone collapse.

Treatment with Recombinant Decorin in Rat Models of SCI Supports Therapeutic Potential

Recombinant human Decorin (rh-Decorin) has been shown to inhibit inflammation, glial scar formation and CSPG expression, and may promote axonal growth across the injury interface after acute spinal cord injury (Wu, Li et al. 2013, Ahmed, Bansal et al. 2014). These data show that Decorin treatment in animal models commenced immediately after spinal cord injury, inhibits TGF-β1/2-mediated invasion of inflammatory cells, scar deposition and cavitation and that later, during the consolidation phase, regulates ECM remodeling by both the induction of MMP and tissue plasminogen activator (tPA) activity and suppression of TIMP and PAI-1. Moreover, in Decorin-treated mature scars in which acute titers of TGF-β1/2 have declined, scar dissolution appears to be induced by MMP/tPA mediated fibrolytic activities and enhanced by depressed levels of TIMP and PAI-1 activity.

Decorin suppressed acute scarring (fibrogenesis) and wound cavitation, and induced dissolution of mature scar tissue (fibrolysis) in dorsal funicular lesion (DFL) SCI model system of the spinal cord in adult rats (Wu, Li et al. 2013, Ahmed, Bansal et al. 2014). DFL cavity treatment with recombinant Decorin suppresses inflammation and scar deposition in the acute and subacute phases of the CNS injury response in rat model of SCI and also contributes to dissolution of the mature scar following SCI. Decorin treatment of spinal cord injury (Esmaeili, Berry et al. 2014, Ahmed, Bansal et al. 2014).

Additionally, Decorin promoted axonal regrowth in both acute and chronic experiments. In both cases, axons were absent in PBS-treated DFL, but present in Decorin-treated DFL.

OPC1 Treatment in Non-Clinical Models of SCI Demonstrates Similar Results to that Seen with Decorin Treatment

In five studies (Table 16) of OPC1 transplant into rodent models of SCI, a statistically significant reduction of cavitation area was observed in OPC1-treated animals, as compared to animals injected with control vehicle (HBSS or IsoLyte plus Human Serum Albumin). In these studies, axonal regrowth through the SCI lesion was seen in all OPC1-treated animals but not in the control animals. Tabulated results of these studies are shown below. The rat model of SCI injury that was used closely emulates the damage and outcomes seen in human after a contusion or crush injury of the cervical spinal cord. For rat model treatment time windows after SCI injury are defined as: Day 1-7: acute stage; Day 7-14: subacute stage; and over 14 days: chronic.

TABLE 16
Summary of preclinical studies demonstrating similar results seen with Decorin treatment

| Study | Timepoint | Study Type | Results |
|---|---|---|---|
| P002-2011 (Seq. 0073) | 4 months | Efficacy study in rats (treated 7- | 86% Reduction in |
| | | 8 days after spinal cord | cavitation area over |
| | | contusion) | control |
| 1058-015 (Seq. 0073) | 9 months | Tox study in rats (treated 6-8 | 4% of OPC1 treated |
| | | days after spinal cord | and 63% of controls |
| | | contusion) | had measurable areas |
| | | | of cavitation |
| 1058-021 (Seq. 0073) | 9 months | Tox study in rats (treated 6-8 | 4% of OPC1 treated |
| | | days after spinal cord | and 58% of controls |
| | | contusion) | had measurable areas |
| | | | of cavitation |
| 2237-001, (Seq. 0100) | 9 months | Tox study in rats (treated 6-8 | 5% of OPC1 treated |
| | | days after spinal cord | and 84% of control |
| | | contusion) | had measurable areas |
| | | | of cavitation |
| SP01-2016 (Seq. 0094) | 2 months | Study comparing timing of | 4-5 fold reduction of |
| | | transplantation (1 week, 2 | cavitation for OPC1 |
| | | weeks, 3 weeks) (treated 2-3 | treated over control at |
| | | weeks after spinal cord | 1 and 2 weeks, little |
| | | contusion) | cavitation reduction |
| | | | for OPC1 treated at 3 |
| | | | weeks over control |

Staining for the presence of myelinated axonal fibers performed in the first four studies listed above has shown the presence of myelinated axonal fibers traversing the lesion area in animals treated with OPC1 but not in the animal treated with the control.

The results observed in the OPC1 animal studies demonstrate very similar anatomical outcomes to that seen in the studies where Decorin infused implants were transplanted into animals with spinal cord injuries as described above (Wu, Li et al. 2013, Ahmed, Bansal et al. 2014). Thus, the anatomical effects observed in the nonclinical efficacy studies of OPC1 transplantation into SCI injury may be attributed, at least in part, to the secretion of Decorin.

### SUMMARY

The current knowledge, as presented above, on the positive role that Decorin plays in attenuating the damage in the SCI cavity and the nonclinical studies results in Decorin-treated and OPC1-treated models of SCI justifies its utilization as a potency indicator for OPC1. It is projected that the ability of OPC1 cells to produce and secrete Decorin is one of the key therapeutic effects of OPC1, by positively modulating scarring and axon regrowth inhibiting processes. Therefore, we intend to use a qualified Decorin assay to be included in the panel of release testing for the new, improved OPC1 production process.

### REFERENCES FOR EXAMPLE 10

Ahmed, Z., D. Bansal, K. Tizzard, S. Surey, M. Esmaeili, A. M. Gonzalez, M. Berry and A. Logan (2014). “Decorin blocks scarring and cystic cavitation in acute and induces scar dissolution in chronic spinal cord wounds.” Neurobiol Dis 64: 163-176.
Bradbury, E. J. and E. R. Burnside (2019). “Moving beyond the glial scar for spinal cord repair.” Nat Commun 10(1): 3879.
Davies, J. E., X. Tang, J. C. Bournat and S. J. Davies (2006). “Decorin promotes plasminogen/plasmin expression within acute spinal cord injuries and by adult microglia in vitro.” J Neurotrauma 23(3-4): 397-408.
Esmaeili, M., M. Berry, A. Logan and Z. Ahmed (2014). “Decorin treatment of spinal cord injury.” Neural Regen Res 9(18): 1653-1656.
Goncalves, M. B., Y. Wu, E. Clarke, J. Grist, C. Hobbs, D. Trigo, J. Jack and J. P. T. Corcoran (2019). “Regulation of Myelination by Exosome Associated Retinoic Acid Release from NG2-Positive Cells.” J Neurosci 39(16): 3013-3027.
Gubbiotti, M. A., S. D. Vallet, S. Ricard-Blum and R. V. Iozzo (2016). “Decorin interacting network: A comprehensive analysis of decorin-binding partners and their versatile functions.” Matrix Biol 55: 7-21.
Hill, L. J., R. J. A. Moakes, C. Vareechon, G. Butt, A. Ng, K. Brock, G. Chouhan, R. C. Vincent, S. Abbondante, R. L. Williams, N. M. Barnes, E. Pearlman, G. R. Wallace, S. Rauz, A. Logan and L. M. Grover (2018). “Sustained release of decorin to the surface of the eye enables scarless corneal regeneration.” NPJ Regen Med 3: 23.
Minor, K., X. Tang, G. Kahrilas, S. J. Archibald, J. E. Davies and S. J. Davies (2008). “Decorin promotes robust axon growth on inhibitory CSPGs and myelin via a direct effect on neurons.” Neurobiol Dis 32(1): 88-95.
Nastase, M. V., A. Janicova, H. Roedig, L. T. Hsieh, M. Wygrecka and L. Schaefer (2018). “Small Leucine-Rich Proteoglycans in Renal Inflammation: Two Sides of the Coin.” J Histochem Cytochem 66(4): 261-272.
Wu, L., J. Li, L. Chen, H. Zhang, L. Yuan and S. J. Davies (2013). “Combined transplantation of GDAs(BMP) and hr-decorin in spinal cord contusion repair.” Neural Regen Res 8(24): 2236-2248.

Example 11—Comparability of GPOR OPC1 Cells and LTCOPC1 OPC1 Cells

Some of the process for making the cells and the OPC1 product, such as some of those used in the clinical studies described in the Examples herein, are referred to as Geron process and Geron cells or GPOR. The new processes described in this and other Examples herein are used for making the LCTOPC1 product. The LCTOPC1 process described herein is the process used for current GMP production of cells for clinical use. Data will be provided in this Example, supporting comparability between these manufacturing processes.

### LIST OF ABBREVIATIONS

ASIA American Spinal Injury Association Impairment
bFGF Basic FGF
BMP Bone Morphogenic Protein
CMC Chemistry Manufacturing Controls
CS10 CryoStor 10
DP Drug Product
ECM Extracellular Matrix
FCM Flow Cytometry
GMP Good manufacturing Process
GPM Glial Progenitor Medium
GPOR Geron Process of Records
hESC Human Embryonic Stem Cells
hsFGF Heat-Stable FGF
IPC In-process Controls
MCB Master Cell Bank
NB Neurobody
NLI Neurological Level of Injury
OCB Original Cell Bank
OL Oligodendrocytes
POC Proof-of-Concept
QC Quality Control
RA Retinoic Acid
R&D Research and Develoment
rhEGF Recombinant Human EGF
RL Risk Level
RMAT Regenerative Medicine Advanced Therapy
SCI Spinal Cord Injury
TAI Thaw-and-Inject
UEMS Upper Extremity Motor Score
WCB Working Cell Bank

LCTOPC1 (OPC1), previously referred to as GRNOPC1 and then AST-OPC1, is an oligodendrocyte progenitor cell population derived from the H1 hESC line intended for one-time administration for the treatment of subacute spinal cord injury (SCI). OPC1 has been shown in pre-clinical studies to produce neurotrophic factors, migrate in the spinal cord parenchyma, stimulate vascularization, and induce remyelination of denuded axons, all of which are essential functions of oligodendrocyte progenitors and are important for survival, regrowth and function of axons.

Clinical evaluation of LCTOPC1 was initiated in 2010 by Geron Corporation. The first clinical trial was a Phase 1 safety study (NCT01217008) in which a low dose of 2×10⁶ OPC1 cells was injected into the lesion site of subjects with subacute, neurologically complete thoracic spinal T3-T11 injuries. A total of 5 subjects out of the planned 8 received OPC1 as part of the original Phase 1 CP35A007 safety study from October 2010 through November 2011.

In 2014, a Phase 1/2a study (NCT02302157) dose escalation of OPC1 in subjects with subacute sensorimotor complete (American Spinal Injury Association Impairment Scale A (ASIA Impairment Scale A)), Single Neurological Level (SNL) from C5 to C7 cervical spinal cord injuries was initiated, with the later expansion of the study to patients with a C4 Neurological Level of Injury (NLI) if the Upper Extremity Motor Score (UEMS)≥1 and changing the dosing window from 14-30 days to 21-42 days post-spinal cord injury. A total of 25 subjects across 5 cohorts were enrolled in the AST-OPC-01 study and received a single administration of OPC1 cells delivered by intra-parenchymal injection into the spinal cord injury site using a Syringe Positioning Device, during a dedicated surgical procedure. The enrollment for AST-OPC-01 study was completed in December 2017 and reported in December 2020.

Briefly, the origin of the new Master Cell Bank (MCB) is the H1 Bank Lot. No. MCBH101. MCBH101 was manufactured by Geron directly from the H1 Original Cell Bank (OCB) in 2009. It was manufactured in feeder-free conditions using well-defined raw materials, new culturing system and harvesting procedure, and cryopreserved by an hESC-customized cryopreservation process. In addition, the method for assessment of H1 hESC pluripotency was optimized. The new WCB originated from the new MCB and was expanded in tissue culture for 4 passages, while maintaining hESC characteristics, and then cryopreserved. The WCB will provide the starting material for LCTOPC1 manufacturing.

The purpose of this Example is also to present the scientific data generated during the development of LCTOPC1 CMC program. The provided information includes the development plans for LCTOPC1 with regards to preliminary comparability results based on R&D runs of the improved manufacturing process, comparability between the GPOR and LCT R&D manufactured material, introduction of a new proposed potency assay, review of the OPC1 safety status based on the GPOR in vivo data and reanalysis of GPOR lots, utilizing improved analytical methods.

Rationale for Process Improvements

OPC1 is an investigational drug studied in a Phase 1 and a Phase 1/2a spinal injury clinical studies using OPC1 clinical lots produced by Geron Inc. Geron's (GPOR) manufacturing process was originally developed in the early 2000s. At that time, well-defined and cell therapy grade reagents and materials were not widely available. As such, Stage 1 of the manufacturing process included the propagation of H1 embryonic cells on Matrigel™, an animal derived Extracellular Matrix (ECM), collagenase, and manual scraping of the culture dish surface for harvesting, passaging and expansion of the H1 embryonic stem cells.

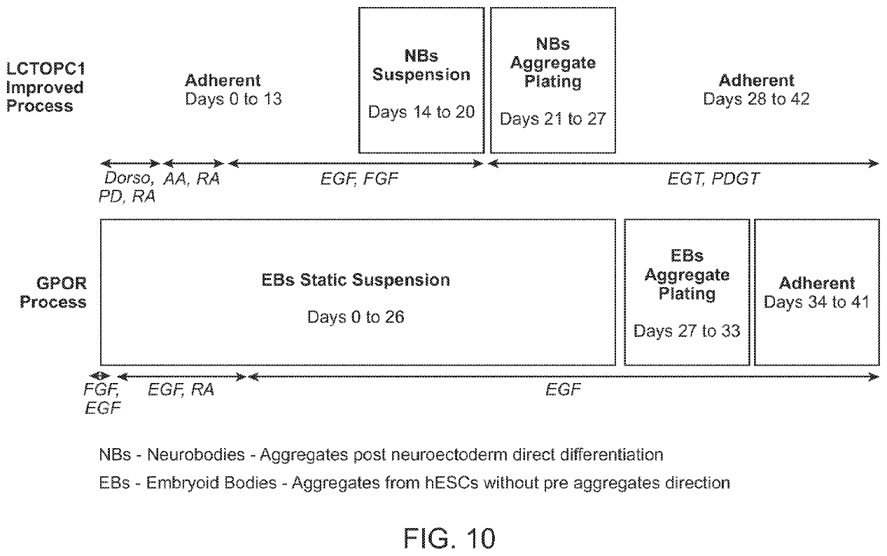

Furthermore, the GPOR manufacturing process was based on a poorly controlled differentiation process, driven by three guiding molecules. Most of the differentiation process occurred in cell aggregates starting directly from pluripotent H1 cells, in the form of Embryoid bodies (day 0 to day 26, FIG. 10), which have a strong susceptible to spontaneous differentiation. From day 27 on, the differentiation was completed on Matrigel™ coated adherent surface for oligodendrocyte progenitor expansion and maturation. The GPOR manufacturing process had low yields, and key quality attributes defined by purity/impurity/non-targeted cell population markers exhibited limited reproducibility. Additionally, the final cryopreserved product required upon thawing, washing and formulation preparation prior to administration.

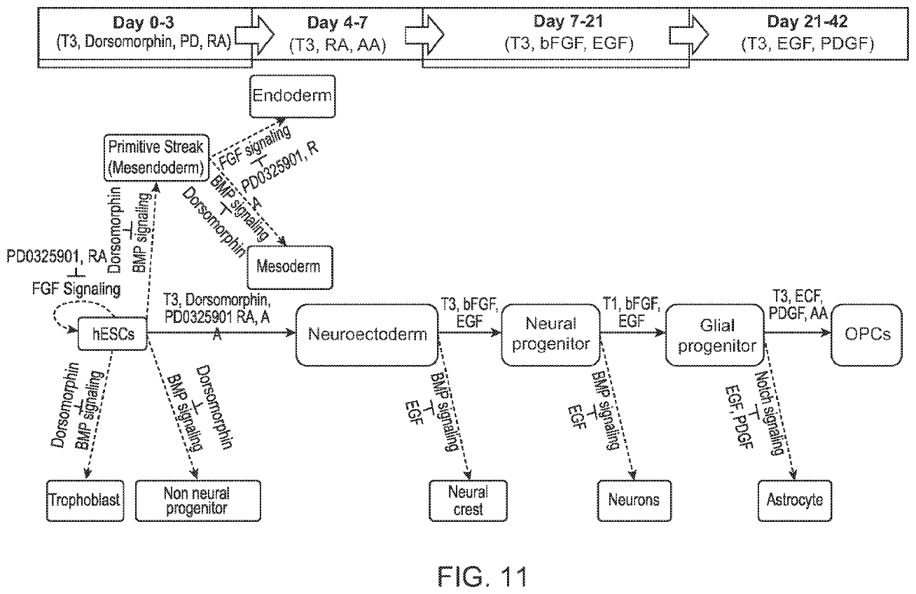

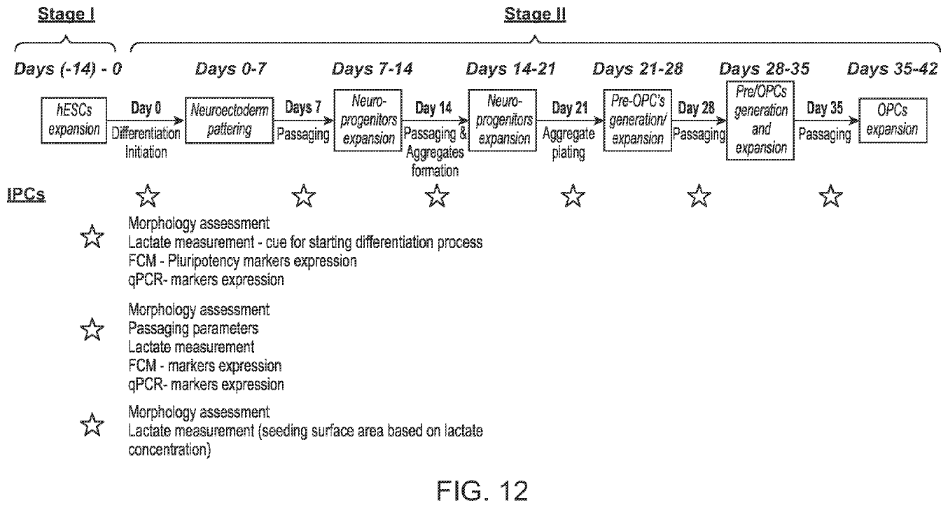

The development of the improved manufacturing process focused on a more controlled directed differentiation of H1 towards OPC, guided by a specific sequence of growth factors and small molecules to inhibit or direct differentiation pathways using cell therapy grade materials when possible (as detailed in FIG. 11). Moreover, the new process reduces the lengthy aggregate phase used by Geron, from 26 days directly from pluripotent cell state, which is prone to spontaneous aberrant differentiation, to 7 days, following 14 days of monolayer directed differentiation of H1 cells into neuroectoderm, reducing the possibility for spontaneous differentiation in the aggregates phase. The GPOR Vs. LCTOPC1 differentiation processes are summarized in FIG. 10. The biological rationale for the signaling sequence of inducing and inhibitory factors of the improved differentiation process is described in FIG. 11. Additionally, new in-process controls (IPCs) were added to better monitor and characterize the differentiation process, as detailed in FIG. 12.

Materials used to manufacture of OPC1 cells (both the original GPOR and the modified processes) are summarized in Table 17.

TABLE 17
Materials used during the production of
OPC cells (GPOR and LCTOPC1 processes).

| | Parameter | GPOR Process | LCTOPC1 Process |
|---|---|---|---|
| Stage I - hESCs | Culturing System | XVIVO 10 Medium | mTeSR Medium |
| H1 expansion | | Collagenase/mechanical | ReleSR |
| | | scraping Matrigel | rhLaminin |
| Stage II - hESCs | Differentiation | GPM (DMEM/F12, T3, B-27) | |
| H1 differentiation | Medium | | |
| into OPC | Coating Reagent | Matrigel | rhLaminin |
| | Growth and | rhEGF | Dorsomorphin |
| | Differentiation | rhbFGF | PD0325901 |
| | Factors | Retinoic Acid (RA) | Retinoic Acid (RA) |
| | | | Ascorbic Acid (AA) |
| | | | rhEGF |
| | | | heat stable rhbFGF |
| | | | ROCK Inhibitor |
| | | | PDGF-A |

Overview of the Lctopc1 Manufacturing Process

Stage I—H1 Expansion

Pluripotent H1 cells are thawed and cultured for 12-15 days on laminin-coated vessels in mTeSR Plus Medium. The cells are passaged and expanded using a non-enzymatic reagent ReLeSR (as described for the MCB and WCB hESC culturing).

During the expansion, the cells are morphologically assessed, and at the end of 3 passages (before the initiation of differentiation process), hESC pluripotency is evaluated by flow cytometry-based

Stage II—H1 Differentiation into OPC1

From day 0 of differentiation until the end of the process, the cells are cultured in Glial Progenitor Medium (GPM)—which is DMEM/F-12 supplemented with B27 and T3.

Day 0-7—on day 0, when the H1 culture reaches the required criteria which is defined by lactate concentration and cell morphology, the differentiation process is initiated by changing the medium for the expanded pluripotent hESC cultured on laminin-coated vessels as follows. On days 0-3, GPM medium is supplemented with Retinoic Acid (RA), Dorsomorphin and PD0325901, in order to direct the differentiation process towards the neuroectoderm pathway (Kudoh, Wilson et al. 2002). Dorsomorphin inhibits Bone Morphogenic Protein (BMP) signaling (SMAD pathway) and therefore inhibits mesoderm and trophoblast differentiation (Li and Parast 2014). PD0325901 inhibits downstream bFGF signaling at MEK1 and MEK2, and inhibits pluripotency and endoderm differentiation (Sui, Bouwens et al. 2013). In summary, inhibition of pluripotency, endoderm, mesoderm and trophoblast formation in addition to activation of the RA signaling pathway, promotes neural tube (neuroectoderm) formation (Watabe and Miyazono 2009, Sui, Bouwens et al. 2013, Li and Parast 2014, Patthey and Gunhaga 2014, Janesick, Wu et al. 2015). On days 4-7, the culture is supplemented with Retinoic Acid and Ascorbic Acid to continue neuroectoderm differentiation induction (Duester 2008).

Day 7-14—On day 7 the cells are enzymatically harvested using TrypLE Select, and then seeded as a monolayer culture from day 7 to day 14 on laminin-coated vessels and cultured in GPM supplemented with rhEGF, hsFGF and ROCK inhibitor (ROCK Inhibitor only for the first 48 hours) (Hu, Du et al. 2009, Patthey and Gunhaga 2014, Zheng, Li at al. 2018).

Day 14-21—On Day 14, in order to promote neurobody (NB) aggregate formation, the cells are enzymatically harvested using TrypLE Select, and cultured for a week as a dynamic suspension culture in GPM supplemented with ROCK inhibitor (for the first 48 hours), and rhEGF and hs-rhFGF throughout.

Day 21-42—On Day 21 the aggregates are plated back as an adherent culture on laminin-coated vessels in GPM supplemented with rhEGF and PDGF (Ota and Ito 2006, Koch, Lehal et al. 2013), and then on Day 28, the cells are harvested enzymatically using TrypLE Select, and expanded as an adherent culture on laminin-coated vessels in GPM supplemented with rhEGF and PDGF until days 35-42 for final expansion and maturation, with enzymatic passaging every ˜7 days using TrypLE Select.

At the end of the expansion process, the OPC1 cells are harvested using TrypLE Select and cryopreserved in CryoStor®10 (CS10; BioLife Solutions, Inc.) cryopreservation solution as a Thaw-and-Inject (TAI) formulation. The LCTOPC1 production process flow is depicted in FIG. 12.

In-Process Control tests are performed at every key step during the differentiation process of hESC to OPC1, as depicted in FIG. 12. Biomarker proteins and mRNA expression are assessed using multicolor Flow Cytometry (FCM) and qPCR methods (respectively). The cells are tested for the expression of OPC1, epithelial, mesodermal, astrocytes and neuronal biomarkers, and residual hESC. In addition, viability, cell yield and metabolic activity (e.g., lactate) are assessed during the process. Lactate concentration is used as indicator for initiating differentiation starting on day 0, and on day 21 as a surrogate to cell counting in order to determine the surface area required for aggregate plating for pre-OPC generation and expansion.

Proposed Cmc Comparability Testing

OPC1 will be manufactured according to the improved process, released according to revised release parameters, and cryopreserved. LCTOPC1 DP will be compared to Geron's manufactured representative batches and characterized with the updated analytical methods used for the release of the OPC1 manufactured via the new process. The plan will include testing of attributes used as release criteria for GPOR plus additional markers that were identified. The suggestion for comparison is based on quality attributes that characterize the Drug Product as described in Table 18.

The side-by-side comparison between LCTOPC1 and GPOR OPC1 batches will be based on statistical analysis, calculating the expected range for quantitative measurements of the quality attributes from GPOR OPC1 batches. The values of those quality attributes measured in LCTOPC1 batches will be assessed in relation to those expected ranges for the quality attributes tested. The comparability data analysis is expected to establish reproducible release criteria for the LCTOPC1 process and demonstrate that LCTOPC1 has low batch to batch variability.

The tested quality attributes will include viability, identity/purity, impurity/non-targeted population, gene profiling, and function/potency assays for 2-3 representative GPOR and LCT OPC1 batches each.

Suggested comparability quality attributes are as follows: Viability—a critical quality attribute of any live cell drug product; Identity/Purity—assessment of characteristic oligodendrocyte progenitor cell protein markers: NG2, GD3, PDGFRα and PDGRFβ; Non-targeted cells population/impurities—(i) Residual H1 hESC from starting material—human embryonic stem cells protein markers TRA-1-60 and Oct-4 as a potential source for teratogenic agents combined in a multi-color Flow Cytometry test. TRA-1-60 and Oct-4 are commonly known and used markers for embryonic stem cells and were used previously as an OPC1 release criteria, and (ii) Assessment of potential aberrant differentiation paths (Epithelial cells protein markers: Keratin 7, Claudin-6, EpCAM and CD49f known epithelial markers, Mesodermal cells protein markers CXCR4 and CD56 as known mesodermal and cartilage markers, Astrocytes cells protein marker GFAP as known astrocytes marker, Neuronal phenotype cells protein marker b-Tubulin 3 as known neuronal marker, Mesenchymal cells mRNA OLR1 that induces epithelial-mesenchymal transition, Endoderm cells mRNA markers of FOXA2, SOX17 and AFP as known endodermal markers, and does not include previously used Nestin and α-actinin attributes, since new data indicate that Nestin is not specific for OPC, but rather a marker for NSC and other cell types and α-actinin can be effectively replaced by combination of CXCR4/CD56. CXCR4 is expressed in definitive endoderm and mesoderm); In-vitro Cells Function/Potency—(i) Decorin secretion—a small secreted cellular matrix proteoglycan, as a potency indicator for OPC1. Decorin expressed by neurons and astrocytes in the central nervous system attenuates scar tissue formation, inhibits cavitation and promotes wound healing. The detailed rationale for the decorin secretion as a proposed potency assay is discussed in Example 10. (ii) OPC1 cells migration in response to platelet-derived growth factor-BB (PDGF-BB) which is important for cell motility at the injury site to ensure broadest anatomical coverage from a single injection location in the spine. (iii) Development of new potency test for assessment of the maturation and myelinization potential of OPC1. This assay is based on an essential function of OPC1 cells-remyelination of denuded axons. In this assay, OPC1 cells are thawed and plated in a specific media in a 3D tissue culture environment (e.g., Matrigel or Nanofiber tubes) that should induce the maturation of OPC1 into Oligodendrocytes (OL).

The 3D environment nanotube mimics denuded axons in order to induce myelinization activity by OPC1 cells in a simple in-vitro setting. The assay will measure secretion of proteins associated with OL function (MBP and decorin) and will test OL protein markers (MBP, O4, MAG, MOG) expression by immunocytochemistry. The assay is currently being developed as a Proof-of-Concept (POC) and will be established if it proves to be robust enough.

The proposed test panel to be used to examine GPOR OPC1 and LCTOPC1 along with proposed release criteria for LCTOPC1 can be seen below in Table 18.

TABLE 18
The proposed test panel and rationale used for process development, release and comparability.

| | | Current | | | | |
|---|---|---|---|---|---|---|
| | | Marker | | | Current Proposed | |
| | | (GPOR | Proposed | Justification | Criteria | |
| Test | Method | OPC1) | Marker/s | and Reference | (based on LCTOPC1) | |
| Viability, | NC-200 | % of viable | % of viable | Critical attribute | ≥90% | |
| Strength | | population | population | to assess the | | |
| | | | | quality of the | | |
| | | | | cell product | | |
| Biosafety | | | | | | |
| Sterility | <USP71> | NA | NA | NA | No growth | |
| Mycoplasma | <USP63> | NA | NA | NA | No growth | |
| Endotoxin | <USP85> | NA | NA | NA | <2 | EU/ml |
| Identity | | | | | | |
| OPC | Flow | % NG2+ | % GD3+ | Known Pre-OPC | ≥80% | |
| | Cytometry | % Nestin+ | | marker | | |
| | | | % NG2+ | Known OPC | ≥80% | |
| | | | | marker | | |
| | | | % PDGFRa+ | Known OPC | ≥90% | |
| | | | | marker | | |
| | | | % PDGFRβ+ | Essential for | ≥90% | |
| | | | | maturation of | | |
| | | | | oligodendrocytes | | |
| Non-targeted cell populations | | | | | | |
| Epithelial | Flow | % Keratin 7+ | % Keratin 7+ % | Known epithelial | ≤4% | |
| | Cytometry | | of Claudin-6+ | markers | ≤2% | |
| | | | % of EpCAM+ | | ≤2% | |
| | | | % CD49f | | ≤4% | |
| hESCs | | % TRA-1- | % TRA-1- | Teratogenic | ≤0.1% | |
| residual | | 60+/Oct4+ | 60+/Oct4+ | potential | | |
| Mesoderm | | α-Actinin+ | % | Known | ≤1% | |
| | | | CXCR4+/CD56+ | mesodermal and | | |
| | | | | cartilage marker | | |
| Astrocytes | | GFAP | % GFAP | Known astrocytes | ≤1% | |
| | | | | marker | | |
| Test | | | Proposed | Justification | Current Proposed | |
| | | | [symbol] | and Reference | | |
| Neuronal | | | % b-Tubulin 3+ | Known neuronal | ≤4% | |
| | | | | marker | | |
| Mesenchymal | | | OLR1 | OLR1 induces | Comparable/lower than | |
| | | | | epithelial- | GPOR OPC-1 | |
| | | | | mesenchymal | | |
| Endoderm | | AFP | FOXA2, SOX17, | Known | Comparable/lower than | |
| | | | AFP | endodermal | GPOR OPC-1 | |
| | | | | markers | | |
| Function/Potency | | | | | | |
| Decorin | ELISA | Decorin | Decorin | Attenuates scar | ? 25 | ng/ml |
| Secretion | | (ng/ml) | (ng/ml) | tissue, blocks | | |
| | | | | cavitation, | | |
| | | | | promotes wound | | |
| | | | | healing | | |
| Cell | NC-200 | NA | % Migrated | Potential mode | ? 30% (+PDGFI313) | |
| Migration | | | (PDGF-induced) | of action; | | |
| | | | | promote healing | | |
| | | | | in tissue | | |

[symbol]

indicates data missing or illegible when filed

Preliminary comparability of R&D LCTOPC1 batches, from representative runs are presented below in Tables 19-23.

TABLE 19
Comparability data from representative GPOR OPC1 and LCTOPC1
R&D Runs - OPC1 Identity profile by flow cytometry.

| | | | | | | LCTOPC1 | | |
|---|---|---|---|---|---|---|---|---|
| Current | Proposed | | GPOR OPC1 | | | RD | RD | RD |
| Marker | Marker | Justification | M08¹ | M22¹ | M25¹ | 11A2 | 11C | 12D1 |
| % NG2 | % GD3 | Known Pre-OPC marker | 85.4 | 89.9 | 86.4 | 90 | 87.1 | 88 |
| % Nestin | % NG2 | Known OPC marker | 47.8 | 27.8 | 18.2 | 88 | 90.5 | 95.3 |
| | % NG2 (*MG) | | 91.6 | 90.7 | 91.2 | 93.9 | 91.3 | 92.7 |
| | % PDGFR_{α} | Known OPC marker | 42.3 | 52.2 | 55.8 | 98.9 | 98.4 | 99.2 |
| | % PDGFR_{α} (*MG) | | 79.7 | 79.9 | 87 | 99 | 99 | 99.3 |
| | % NG2/PDGFR | Known OPC markers | 27 | 16.2 | 11.8 | 86.5 | 89.1 | 94.9 |
| | % NG2/PDGFRα | | 78.5 | 78 | 83.8 | 93.4 | 91.1 | 92.5 |
| | % PDGFRβ | Essential for maturation of | 92.7 | 87.1 | 91.6 | 99.1 | 98.7 | 99.1 |
| | | oligodendrocytes | | | | | | |

*MG—Cells are seeded on Matrigel overnight before FCM analysis. GPOR OPC1 formulation and freezing reduces NG-2 and PDGFR-α expression.
¹Clinical batch

TABLE 20
Comparability data from representative GPOR and LCTOPC1 R&D Runs -
non- targeted/impurities cell population profile by flow cytometry.

| | | | | | | | LCTOPC1 | | |
|---|---|---|---|---|---|---|---|---|---|
| Cell | Current | Proposed | | GPOR OPC1 | | | RD | RD | RD |
| population | Marker | Marker | Justification | M08¹ | M22¹ | M25¹ | 11A2 | 11C | 12D1 |
| Epithelial | % Keratin 7 | % Keratin | Known | 1.04 | 1.47 | 1.88 | 0.25 | 0.38 | 0.36 |
| | | % Claudin- | epithelial | 4.45 | 5.83 | 3.09 | 0.33 | 0.42 | 0.17 |
| | | % EpCAM | markers | 1.17 | 2.21 | 1.37 | 0.62 | 0.72 | 0.99 |
| | | % CD49f | | 24.3 | 21.2 | 23.6 | 0.83 | 1.26 | 2.66 |
| Astrocytes | % GFAP | % GFAP | Known | 1.78 | 3.44 | 1.62 | 0.028 | 0.04 | 1.35 |
| | | | astrocytes | | | | | | |
| Neuronal | % b-Tubulin | % β-Tubulin | Known | 13.7 | 17.9 | 14.3 | 0.51 | 0.29 | 0.87 |
| Phenotype | III | III | Neuronal | | | | | | |
| | | | marker | | | | | | |

¹Clinical batch

TABLE 21
Comparability data from representative GPOR OPC1 and
LCTOPC1 R&D Runs - hESC residuals by flow cytometry.

| | | | | | | | LCTOPC | | |
|---|---|---|---|---|---|---|---|---|---|
| Cell | Current | Proposed | | GPOR OPC1 | | | RD | RD | RD |
| population | marker | marker | Justification | M08¹ | M22¹ | M25¹ | 11A2 | 11C | 12D1 |
| hESCs | % TRA-1- | % TRA-1- | Teratogenic | 0.01 | 0.00 | 0.00 | 0.0 | 0.00 | 0.00 |
| hESCs | % TRA-1- | % TRA-1- | Teratogenic | 0.41 | 0.49 | 0.44 | 0.0 | 0.04 | 0.01 |

¹Clinical batch

TABLE 22
Comparability data from representative GPOR OPC1 and LCTOPC1
R&D Runs - non- targeted/impurities cell population gene
profile by qPCR (relative to GPOR OPC1 M08).

| Cell | Current | Proposed | | GPOR OPC1 | | | LCTOPC1 | | |
|---|---|---|---|---|---|---|---|---|---|
| populatio | Marker | Marker | Justificatio | M08 | M22 | M25 | RD | RD | RD |
| Endoderm | AFP | AFP | Known | 1.00 | 1.35 | 1.11 | N | N | ND |
| | | FOXA2 | endoderm | ND | ND | ND | N | N | ND |
| | | SOX17 | genes | ND | ND | ND | ND | ND | ND |
| Mesenchymal | OLR1 | OLR1 | OLR1 | 1.00 | 1.31 | 1.77 | ND | ND | ND |
| | | | induces | | | | | | |
| | | | epithelial- | | | | | | |
| | | | mesenchymal | | | | | | |
| | | | transition | | | | | | |

*ND—Not Detected
¹Clinical batch

TABLE 23
Comparability data from representative GPOR OPC1 and LCT OPC1 R&D Runs - in
vitro function as determined by decorin secretion and migration assays.

| | | GPOR OPC1 | | | LCTOPC1 | | |
|---|---|---|---|---|---|---|---|
| Test | Justification | M08¹ | IM22¹ | M25¹ | RD | RD | RD |
| Decorin Secretion | Attenuates scar tissue, | 25.6 | 27.61 | 23.75 | 36.67 | 29.12 | 29.78 |
| (ng/mL) | blocks cavitation, | | | | | | |
| Cells Migration | Potential mode of action; | 36 | 47 | 33 | 49 | 44 | 35 |
| (% migrated, | promote healing in tissue | | | | | | |
| PDGF-bb induced) | | | | | | | |

¹Clinical batch

Review of Ectopic Tissue Formation Assessment as a Release Criterion for Opc1 Drug Product

This analysis revealed certain correlations between the available data from GLP tox studies of Geron GMP batches to the expression level of purity and impurities/non targeted cell population specific markers.

The retesting results and analysis show correlation between impurities and batch failure in vivo, indicating that LCTOPC1, characterized by significant lower levels of impurities and high purity profile, has a greatly reduced likelihood of cyst formation in vivo than does GPOR OPC1.

### SUMMARY

Preliminary comparability data of representative GPOR and LCTOPC1 batches demonstrate: GPOR OPC1 and R&D LCTOPC1 demonstrate similar OPC1 identity/purity profile profiles, except for the PDGFR-α biomarker which is higher in R&D LCTOPC1 and may result from the improved OPC1 manufacturing process (directed differentiation). LCTOPC1 has lower levels of impurities from epithelial, astrocytes and neuronal non-targeted cells, compared to GPOR OPC1. Both LCTOPC1 and GPOR OPC1 have very rarely detectable residual hESC (detected by % TRA-1-60+/Oct4+); however, GPOR OPC1 has a higher percentage of multipotent cells compared to LCTOPC1 as demonstrated by populations of cells exhibiting TRA-1-60+/Oct4- and CD49f+. LCTOPC1 has lower levels of endoderm and mesenchymal non-targeted cells gene expression, compared to GPOR OPC1. LCTOPC1 and GPOR OPC1 demonstrate similar decorin secretion and migration capacity.

Conclusions: the data accumulated to date show that LCTOPC1 drug product manufactured with an improved process, generated from a defined cell banking system by a highly-reproducible and tightly-controlled differentiation process and monitored with updated analytical methods, presents similar essential quality attributes to GPOR OPC1, but benefits from overall higher expression of purity markers and lower expression of an impurity/non-targeted population markers. Thus, potentially increasing the safety profile for LCTOPC1 drug product.

### REFERENCES FOR EXAMPLE 11

Duester, G. (2008). “Retinoic acid synthesis and signaling during early organogenesis.” Cell 134(6): 921-931.
Hu, B. Y., Z. W. Du, X. J. Li, M. Ayala and S. C. Zhang (2009). “Human oligodendrocytes from embryonic stem cells: conserved SHH signaling networks and divergent FGF effects.” Development 136(9): 1443-1452.
Janesick, A., S. C. Wu and B. Blumberg (2015). “Retinoic acid signaling and neuronal differentiation.” Cell Mol Life Sci 72(8): 1559-1576.
Koch, U., R. Lehal and F. Radtke (2013). “Stem cells living with a Notch.” Development 140(4): 689-704.
Kudoh, T., S. W. Wilson and I. B. Dawid (2002). “Distinct roles for Fgf, Wnt and retinoic acid in posteriorizing the neural ectoderm.” Development 129(18): 4335-4346.
Li, Y. and M. M. Parast (2014). “BMP4 regulation of human trophoblast development.” Int J Dev Biol 58(2-4): 239-246.
Ota, M. and K. Ito (2006). “BMP and FGF-2 regulate neurogenin-2 expression and the differentiation of sensory neurons and glia.” Dev Dyn 235(3): 646-655.
Patthey, C. and L. Gunhaga (2014). “Signaling pathways regulating ectodermal cell fate choices.” Exp Cell Res 321(1): 11-16.
Sui, L., L. Bouwens and J. K. Mfopou (2013). “Signaling pathways during maintenance and definitive endoderm differentiation of embryonic stem cells.” Int J Dev Biol 57(1): 1-12.
Watabe, T. and K. Miyazono (2009). “Roles of TGF-beta family signaling in stem cell renewal and differentiation.” Cell Res 19(1): 103-115.
Zheng, W., Q. Li, C. Zhao, Y. Da, H. L. Zhang and Z. Chen (2018). “Differentiation of Glial Cells From hiPSCs: Potential Applications in Neurological Diseases and Cell Replacement Therapy.” Front Cell Neurosci 12: 239.

All references referred to herein are incorporated by references in their entireties, for all that is taught therein.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure. Therefore, it is intended that the present disclosure not be limited to the particular embodiments disclosed as the best mode contemplated for carrying out the present disclosure, but that the present disclosure will include all aspects falling within the scope and spirit of the appended claims.

### EMBODIMENTS

Embodiment P1. A method of cellular therapy comprising administering to a subject an OPC composition to improve one or more neurological functions in the subject.

Embodiment P2. The method of embodiment P1, wherein the OPC cell population is injected or implanted into the subject.

Embodiment P3. The method of embodiment P1 or P2, wherein the subject has a neurological injury due to spinal cord injury, stroke, or multiple sclerosis.

Embodiment 1. A method of improving one or more neurological functions in a subject having a spinal cord injury (SCI), the method comprising: administering to the subject a first dose of a composition comprising human pluripotent stem cell-derived oligodendrocyte progenitor cells (OPCs); and optionally administering two or more doses of the composition.

Embodiment 2. The method of embodiment 1, further comprising administering to the subject a second dose of the composition.

Embodiment 3. The method of embodiment 1, further comprising administering to the subject a third dose of the composition.

Embodiment 4. The method of any of embodiments 1-3, wherein each administration comprises injecting the composition into the spinal cord of the subject.

Embodiment 5. The method of any of embodiments 1-4, wherein the SCI is a subacute cervical SCI.

Embodiment 6. The method of any of embodiments 1-4, wherein the SCI is a chronic cervical SCI.

Embodiment 7. The method of any of embodiments 1-4, wherein the SCI is a subacute thoracic SCI.

Embodiment 8. The method of any of embodiments 1-4, wherein the SCI is a chronic thoracic SCI.

Embodiment 9. The method of any one of the preceding embodiments, wherein the first dose, second dose, and/or third dose of the composition comprises about 1×10⁶ to about 3×10⁷ OPC cells.

Embodiment 10. The method of any one of the preceding embodiments, wherein the first dose of the composition comprises about 2×10⁶ OPC cells.

Embodiment 11. The method of any one of the preceding embodiments, wherein the first dose or the second dose of the composition comprises about 1×10⁷ OPC cells.

Embodiment 12. The method of any one of the preceding embodiments, wherein the second dose or the third dose of the composition comprises about 2×10⁷ OPC cells.

Embodiment 13. The method of any one of the preceding embodiments, wherein each of the first dose, second dose, and third dose of the composition are administered about 20 to about 45 days after the SCI.

Embodiment 14. The method of any one of the preceding embodiments, wherein each of the first dose, second dose, and third dose of the composition are administered about 14 to about 90 days after the SCI.

Embodiment 15. The method of any one of the preceding embodiments, wherein each of the first dose, second dose, and third dose of the composition are administered about 14 to about 75 days after the SCI.

Embodiment 16. The method of any one of the preceding embodiments, wherein each of the first dose, second dose, and third dose of the composition are administered about 14 to about 60 days after the SCI.

Embodiment 17. The method of any one of the preceding embodiments, wherein each of the first dose, second dose, and third dose of the composition are administered about 14 to about 30 days after the SCI.

Embodiment 18. The method of any one of the preceding embodiments, wherein each of the first dose, second dose, and third dose of the composition are administered about 20 to about 75 days after the SCI.

Embodiment 19. The method of any one of the preceding embodiments, wherein each of the first dose, second dose, and third dose of the composition are administered about 20 to about 60 days after the SCI.

Embodiment 20. The method of any one of the preceding embodiments, wherein each of the first dose, second dose, and third dose of the composition are administered about 20 to about 40 days after the SCI.

Embodiment 21. The method of any one of the preceding embodiments, wherein each of the first dose, second dose, and third dose of the composition are administered between about 14 days after the SCI and the lifetime of the subject.

Embodiment 22. The method of any one of embodiments 2-21, wherein the injection is performed in a caudal half of an epicenter of the SCI.

Embodiment 23. The method of embodiment 22, wherein the injection is about 6 mm into the spinal cord of the subject.

Embodiment 24. The method of embodiment 22, wherein the injection is about 5 mm into the spinal cord of the subject.

Embodiment 25. A method of improving one or more neurological functions in a subject having a spinal cord injury (SCI), the method comprising: administering to the subject a dose of a composition comprising human pluripotent stem cell-derived oligodendrocyte progenitor cells (OPCs).

Embodiment 26. The method of embodiment 25, wherein the dose of the composition comprises about 1×10⁶ to about 3×10⁷ OPC cells.

Embodiment 27. The method of embodiment 26, wherein the dose of the composition comprises about 2×10⁶ OPC cells.

Embodiment 28. The method of any one of embodiments 25-27, wherein the administration of the composition comprises injecting the composition into the spinal cord of the subject.

Embodiment 29. The method of any one of embodiments 25-28, wherein the composition is administered about 7 to about 14 days after the SCI.

Embodiment 30. The method of any one of embodiments 25-29, wherein the injection is performed in a caudal half of an epicenter of the SCI.

Embodiment 31. The method of any one of embodiments 25-30, wherein the injection is about 6 mm into the spinal cord of the subject.

Embodiment 32. The method of any one of embodiments 25-30, wherein the injection is about 5 mm into the spinal cord of the subject.

Embodiment 33. The method of any one of embodiments 25-32 wherein the SCI is a subacute thoracic SCI.

Embodiment 34. The method of any one of embodiments 25-32 wherein the SCI is a chronic thoracic SCI.

Embodiment 35. The method of any one of embodiments 25-32 wherein the SCI is a subacute cervical SCI.

Embodiment 36. The method of any one of embodiments 25-32 wherein the SCI is a chronic cervical SCI.

Embodiment 37. The method of any one of the above embodiments, wherein improving one or more neurological functions comprises an improvement in ISNCSCI exam upper extremity motor score (UEMS).

Embodiment 38. The method of embodiment 37, where in the improvement in UEMS occurs within about 6 months, about 12 months, about 18 months, about 24 months or more after injection.

Embodiment 39. The method of embodiment 37 or 38, wherein the improvement is an increase in UEMS of at least 10%, compared to baseline.

Embodiment 40. The method of any one of the above embodiments, wherein improving one or more neurological functions comprises an improvement in lower extremity motor scores (LEMS).

Embodiment 41. The method of embodiment 40, where in the improvement in LEMS occurs within about 6 months, about 12 months, about 18 months, about 24 months or more after injection.

Embodiment 42. The method of embodiment 37 or 38, wherein the improvement is at least one motor level improvement.

Embodiment 43. The method of embodiment 37 or 38, wherein the improvement is at least two motor level improvement.

Embodiment 44. The method of any one of embodiments 37-43, wherein the improvement is on one side of the subject's body.

Embodiment 45. The method of any one of embodiments 37-43, wherein the improvement is on both sides of the subject's body.

Embodiment 46. The method of any one of the preceding embodiments, wherein the dose of the composition is administered about 14 to about 90 days after the SCI.

Embodiment 47. The method of any one of the preceding embodiments, wherein the dose of the composition is administered about 14 to about 75 days after the SCI.

Embodiment 48. The method of any one of the preceding embodiments, wherein the dose of the composition is administered about 14 to about 60 days after the SCI.

Embodiment 49. The method of any one of the preceding embodiments, wherein the dose of the composition is administered about 14 to about 30 days after the SCI.

Embodiment 50. The method of any one of the preceding embodiments, wherein the dose of the composition is administered about 20 to about 75 days after the SCI.

Embodiment 51. The method of any one of the preceding embodiments, wherein the dose of the composition is administered about 20 to about 60 days after the SCI.

Embodiment 52. The method of any one of the preceding embodiments, wherein the dose of the composition is administered about 20 to about 40 days after the SCI.

Embodiment 53. The method of any one of the preceding embodiments, wherein the dose of the composition is administered between about 14 days after the SCI and the lifetime of the subject.

Embodiment 54. A cell population comprising an increased proportion of cells positive for oligodendrocyte progenitor cell marker NG2 and reduced expression of non-OPC markers CD49f, CLDN6, and EpCAM, wherein the cell population is prepared according to the following method: culturing undifferentiated human embryonic stem cells (uhESC) in Glial Progenitor Medium comprising a MAPK/ERK inhibitor, a BMP signaling inhibitor, and Retinoic Acid to obtain glial-restricted cells; differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

Embodiment 55. The cell population of embodiment 54, for use in treating a thoracic spinal cord injury (SCI) in a subject.

Embodiment 56. The cell population of embodiment 55, wherein the thoracic SCI is a subacute thoracic SCI.

Embodiment 57. The cell population of embodiment 55, wherein the thoracic SCI is a chronic thoracic SCI.

Embodiment 58. The cell population of embodiment 54, for use in treating a cervical spinal cord injury (SCI) in a subject.

Embodiment 59. The cell population of embodiment 58, wherein the cervical SCI is a subacute cervical SCI.

Embodiment 60. The cell population of embodiment 58, wherein the cervical SCI is a chronic cervical SCI.

Embodiment 61. The cell population of any one of embodiments 54-60, wherein the composition is administered via injection to the subject after the SCI.

Embodiment 62. The cell population of embodiment 61, wherein the injection is performed in a caudal half of an epicenter of the SCI.

Embodiment 63. The cell population of embodiment 61, wherein the injection is about 6 mm into the spinal cord of the subject.

Embodiment 64. The cell population of embodiment 61, wherein the injection is about 5 mm into the spinal cord of the subject.

Embodiment 65. The cell population of any one of embodiments 54-64, wherein the injection is performed about 14 to about 90 days after the SCI.

Embodiment 66. The cell population of any one of embodiments 54-64, wherein the injection is performed about 14 to about 75 days after the SCI.

Embodiment 67. The cell population of any one of embodiments 54-64, wherein the injection is performed about 14 to about 60 days after the SCI.

Embodiment 68. The cell population of any one of embodiments 54-64, wherein the injection is performed about 14 to about 30 days after the SCI.

Embodiment 69. The cell population of any one of embodiments 54-64, wherein the injection is performed about 20 to about 75 days after the SCI.

Embodiment 70. The cell population of any one of embodiments 54-64, wherein the injection is performed about 20 to about 60 days after the SCI.

Embodiment 71. The cell population of any one of embodiments 54-64, wherein the injection is performed about 20 to about 40 days after the SCI.

Embodiment 72. The cell population of any one of embodiments 54-64, wherein the injection is performed between about 14 days after the SCI and the lifetime of the subject.

Embodiment 73. A method of improving one or more neurological functions in a subject having a spinal cord injury (SCI), the method comprising: administering to the subject a first dose of the cell population of embodiment 54; administering to the subject a second dose of the cell population; and optionally administering to the subject a third dose of the cell population.

Embodiment 74. The method of embodiment 73, wherein the SCI is a subacute cervical SCI.

Embodiment 75. The method of embodiment 73, wherein the SCI is a chronic cervical SCI.

Embodiment 76. The method of embodiment 73, wherein the SCI is a subacute thoracic SCI.

Embodiment 77. The method of embodiment 73, wherein the SCI is a chronic thoracic SCI.

Embodiment 78. The method of any one of embodiments 73-77, wherein each of the first dose, second dose, and third dose of the composition are administered about 14 to about 90 days after the SCI.

Embodiment 79. The method of any one of embodiments 73-77, wherein each of the first dose, second dose, and third dose of the composition are administered about 14 to about 75 days after the SCI.

Embodiment 80. The method of any one of embodiments 73-77, wherein each of the first dose, second dose, and third dose of the composition are administered about 14 to about 60 days after the SCI.

Embodiment 81. The method of any one of embodiments 73-77, wherein each of the first dose, second dose, and third dose of the composition are administered about 14 to about 30 days after the SCI.

Embodiment 82. The method of any one of embodiments 73-77, wherein each of the first dose, second dose, and third dose of the composition are administered about 20 to about 75 days after the SCI.

Embodiment 83. The method of any one of embodiments 73-77, wherein each of the first dose, second dose, and third dose of the composition are administered about 20 to about 60 days after the SCI.

Embodiment 84. The method of any one of embodiments 73-77, wherein each of the first dose, second dose, and third dose of the composition are administered about 20 to about 40 days after the SCI.

Embodiment 85. The method of any one of embodiments 73-77, wherein each of the first dose, second dose, and third dose of the composition are administered between about 14 days after the SCI and the lifetime of the subject.

What is claimed is:

1. A method of improving one or more neurological functions in a subject having a spinal cord injury (SCI), the method comprising:
administering to the subject a first dose of a composition comprising human pluripotent stem cell-derived oligodendrocyte progenitor cells (OPCs); and optionally administering two or more doses of the composition.

2. The method of claim 1, further comprising administering to the subject a second dose of the composition.

3. The method of claim 1, further comprising administering to the subject a third dose of the composition.

4. The method of any of claims 1-3, wherein each administration comprises injecting the composition into the spinal cord of the subject.

5. The method of any of claims 1-4, wherein the SCI is a subacute cervical SCI.

6. The method of any of claims 1-4, wherein the SCI is a chronic cervical SCI.

7. The method of any of claims 1-4, wherein the SCI is a subacute thoracic SCI.

8. The method of any of claims 1-4, wherein the SCI is a chronic thoracic SCI.

9. The method of any one of the preceding claims, wherein the first dose, second dose, and/or third dose of the composition comprises about 1×10⁶ to about 3×10⁷ OPC cells.

10. The method of any one of the preceding claims, wherein the first dose of the composition comprises about 2×10⁶ OPC cells.

11. The method of any one of the preceding claims, wherein the first dose or the second dose of the composition comprises about 1×10⁷ OPC cells.

12. The method of any one of the preceding claims, wherein the second dose or the third dose of the composition comprises about 2×10⁷ OPC cells.

13. The method of any one of the preceding claims, wherein each of the first dose, second dose, and third dose of the composition are administered about 20 to about 45 days after the SCI.

14. The method of any one of the preceding claims, wherein each of the first dose, second dose, and third dose of the composition are administered about 14 to about 90 days after the SCI.

15. The method of any one of the preceding claims, wherein each of the first dose, second dose, and third dose of the composition are administered about 14 to about 75 days after the SCI.

16. The method of any one of the preceding claims, wherein each of the first dose, second dose, and third dose of the composition are administered about 14 to about 60 days after the SCI.

17. The method of any one of the preceding claims, wherein each of the first dose, second dose, and third dose of the composition are administered about 14 to about 30 days after the SCI.

18. The method of any one of the preceding claims, wherein each of the first dose, second dose, and third dose of the composition are administered about 20 to about 75 days after the SCI.

19. The method of any one of the preceding claims, wherein each of the first dose, second dose, and third dose of the composition are administered about 20 to about 60 days after the SCI.

20. The method of any one of the preceding claims, wherein each of the first dose, second dose, and third dose of the composition are administered about 20 to about 40 days after the SCI.

21. The method of any one of the preceding claims, wherein each of the first dose, second dose, and third dose of the composition are administered between about 14 days after the SCI and the lifetime of the subject.

22. The method of any one of claims 2-21, wherein the injection is performed in a caudal half of an epicenter of the SCI.

23. The method of claim 22, wherein the injection is about 6 mm into the spinal cord of the subject.

24. The method of claim 22, wherein the injection is about 5 mm into the spinal cord of the subject.

25. A method of improving one or more neurological functions in a subject having a spinal cord injury (SCI), the method comprising:
administering to the subject a dose of a composition comprising human pluripotent stem cell-derived oligodendrocyte progenitor cells (OPCs).

26. The method of claim 25, wherein the dose of the composition comprises about 1×10⁶ to about 3×10⁷ OPC cells.

27. The method of claim 26, wherein the dose of the composition comprises about 2×10⁶ OPC cells.

28. The method of any one of claims 25-27, wherein the administration of the composition comprises injecting the composition into the spinal cord of the subject.

29. The method of any one of claims 25-28, wherein the composition is administered about 7 to about 14 days after the SCI.

30. The method of any one of claims 25-29, wherein the injection is performed in a caudal half of an epicenter of the SCI.

31. The method of any one of claims 25-30, wherein the injection is about 6 mm into the spinal cord of the subject.

32. The method of any one of claims 25-30, wherein the injection is about 5 mm into the spinal cord of the subject.

33. The method of any one of claims 25-32 wherein the SCI is a subacute thoracic SCI.

34. The method of any one of claims 25-32 wherein the SCI is a chronic thoracic SCI.

35. The method of any one of claims 25-32 wherein the SCI is a subacute cervical SCI.

36. The method of any one of claims 25-32 wherein the SCI is a chronic cervical SCI.

37. The method of any one of the above claims, wherein improving one or more neurological functions comprises an improvement in ISNCSCI exam upper extremity motor score (UEMS).

38. The method of claim 37, where in the improvement in UEMS occurs within about 6 months, about 12 months, about 18 months, about 24 months or more after injection.

39. The method of claim 37 or 38, wherein the improvement is an increase in UEMS of at least 10%, compared to baseline.

40. The method of any one of the above claims, wherein improving one or more neurological functions comprises an improvement in lower extremity motor scores (LEMS).

41. The method of claim 40, where in the improvement in LEMS occurs within about 6 months, about 12 months, about 18 months, about 24 months or more after injection.

42. The method of claim 37 or 38, wherein the improvement is at least one motor level improvement.

43. The method of claim 37 or 38, wherein the improvement is at least two motor level improvement.

44. The method of any one of claims 37-43, wherein the improvement is on one side of the subject's body.

45. The method of any one of claims 37-43, wherein the improvement is on both sides of the subject's body.

46. The method of any one of the preceding claims, wherein the dose of the composition is administered about 14 to about 90 days after the SCI.

47. The method of any one of the preceding claims, wherein the dose of the composition is administered about 14 to about 75 days after the SCI.

48. The method of any one of the preceding claims, wherein the dose of the composition is administered about 14 to about 60 days after the SCI.

49. The method of any one of the preceding claims, wherein the dose of the composition is administered about 14 to about 30 days after the SCI.

50. The method of any one of the preceding claims, wherein the dose of the composition is administered about 20 to about 75 days after the SCI.

51. The method of any one of the preceding claims, wherein the dose of the composition is administered about 20 to about 60 days after the SCI.

52. The method of any one of the preceding claims, wherein the dose of the composition is administered about 20 to about 40 days after the SCI.

53. The method of any one of the preceding claims, wherein the dose of the composition is administered between about 14 days after the SCI and the lifetime of the subject.

54. A cell population comprising an increased proportion of cells positive for oligodendrocyte progenitor cell marker NG2 and reduced expression of non-OPC markers CD49f, CLDN6, and EpCAM, wherein the cell population is prepared according to the following method:
(i) culturing undifferentiated human embryonic stem cells (uhESC) in Glial Progenitor Medium comprising a MAPK/ERK inhibitor, a BMP signaling inhibitor, and Retinoic Acid to obtain glial-restricted cells;
(iii) differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

55. The cell population of claim 54, for use in treating a thoracic spinal cord injury (SCI) in a subject.

56. The cell population of claim 55, wherein the thoracic SCI is a subacute thoracic SCI.

57. The cell population of claim 55, wherein the thoracic SCI is a chronic thoracic SCI.

58. The cell population of claim 54, for use in treating a cervical spinal cord injury (SCI) in a subject.

59. The cell population of claim 58, wherein the cervical SCI is a subacute cervical SCI.

60. The cell population of claim 58, wherein the cervical SCI is a chronic cervical SCI.

61. The cell population of any one of claims 54-60, wherein the composition is administered via injection to the subject after the SCI.

62. The cell population of claim 61, wherein the injection is performed in a caudal half of an epicenter of the SCI.

63. The cell population of claim 61, wherein the injection is about 6 mm into the spinal cord of the subject.

64. The cell population of claim 61, wherein the injection is about 5 mm into the spinal cord of the subject.

65. The cell population of any one of claims 54-64, wherein the injection is performed about 14 to about 90 days after the SCI.

66. The cell population of any one of claims 54-64, wherein the injection is performed about 14 to about 75 days after the SCI.

67. The cell population of any one of claims 54-64, wherein the injection is performed about 14 to about 60 days after the SCI.

68. The cell population of any one of claims 54-64, wherein the injection is performed about 14 to about 30 days after the SCI.

69. The cell population of any one of claims 54-64, wherein the injection is performed about 20 to about 75 days after the SCI.

70. The cell population of any one of claims 54-64, wherein the injection is performed about 20 to about 60 days after the SCI.

71. The cell population of any one of claims 54-64, wherein the injection is performed about 20 to about 40 days after the SCI.

72. The cell population of any one of claims 54-64, wherein the injection is performed between about 14 days after the SCI and the lifetime of the subject.

73. A method of improving one or more neurological functions in a subject having a spinal cord injury (SCI), the method comprising:
administering to the subject a first dose of the cell population of claim 54;
administering to the subject a second dose of the cell population; and optionally administering to the subject a third dose of the cell population.

74. The method of claim 73, wherein the SCI is a subacute cervical SCI.

75. The method of claim 73, wherein the SCI is a chronic cervical SCI.

76. The method of claim 73, wherein the SCI is a subacute thoracic SCI.

77. The method of claim 73, wherein the SCI is a chronic thoracic SCI.

78. The method of any one of claims 73-77, wherein each of the first dose, second dose, and third dose of the composition are administered about 14 to about 90 days after the SCI.

79. The method of any one of claims 73-77, wherein each of the first dose, second dose, and third dose of the composition are administered about 14 to about 75 days after the SCI.

80. The method of any one of claims 73-77, wherein each of the first dose, second dose, and third dose of the composition are administered about 14 to about 60 days after the SCI.

81. The method of any one of claims 73-77, wherein each of the first dose, second dose, and third dose of the composition are administered about 14 to about 30 days after the SCI.

82. The method of any one of claims 73-77, wherein each of the first dose, second dose, and third dose of the composition are administered about 20 to about 75 days after the SCI.

83. The method of any one of claims 73-77, wherein each of the first dose, second dose, and third dose of the composition are administered about 20 to about 60 days after the SCI.

84. The method of any one of claims 73-77, wherein each of the first dose, second dose, and third dose of the composition are administered about 20 to about 40 days after the SCI.

85. The method of any one of claims 73-77, wherein each of the first dose, second dose, and third dose of the composition are administered between about 14 days after the SCI and the lifetime of the subject.
